(19) United States
(12) Patent Application Publication
(10) Pub. No.: US 2003/0207284 A1
(43) Pub. Date: Nov. 6, 2003

(54) Haplotypes of the UCP2 gene

(76) Inventors: Anne Chew, Brookline, MA (US); R. Rex Denton, Madison, CT (US); Christopher Raleigh Gilson, West Haven, CT (US); Krishnan Nandabalan, Guilford, CT (US); Katie E. Parks, Naugatuck, CT (US)

Correspondence Address: GENAISSANCE PHARMACEUTICALS, 5 SCIENCE PARK, NEW HAVEN, CT, 06511, US

(21) Appl. No.: 10/197,019

(22) Filed: Jul. 16, 2002

Related U.S. Application Data
(63) Continuation-in-part of application No. PCT/US01/02485, filed on Jan. 25, 2001.

(51) Int. Cl. C12Q 1/68

(52) U.S. Cl. 435/6

(57) ABSTRACT

Novel genetic variants of the Uncoupling Protein 2 (Mitochondrial, Proton Carrier) (UCP2) gene are described. Various genotypes, haplotypes, and haplotype pairs that exist in the general United States population are disclosed for the UCP2 gene. Compositions and methods for haplotyping and/or genotyping the UCP2 gene in an individual are also disclosed. Polynucleotides defined by the haplotypes disclosed herein are also described.

### RELATED APPLICATIONS

This application is a continuation-in-part of pending International Application PCT/US01/02485 filed Jan. 25, 2001.

### FIELD OF THE INVENTION

This invention relates to variation in genes that encode pharmaceutically-important proteins. In particular, this invention provides genetic variants of the human uncoupling protein 2 (mitochondrial, proton carrier) (UCP2) gene and methods for identifying which variant(s) of this gene is/are possessed by an individual.

### BACKGROUND OF THE INVENTION

Current methods for identifying pharmaceuticals to treat disease often start by identifying, cloning, and expressing an important target protein related to the disease. A determination of whether an agonist or antagonist is needed to produce an effect that may benefit a patient with the disease is then made. Then, vast numbers of compounds are screened against the target protein to find new potential drugs. The desired outcome of this process is a lead compound that is specific for the target, thereby reducing the incidence of the undesired side effects usually caused by activity at non-intended targets. The lead compound identified in this screening process then undergoes further in vitro and in vivo testing to determine its absorption, disposition, metabolism and toxicological profiles. Typically, this testing involves use of cell lines and animal models with limited, if any, genetic diversity.

What this approach fails to consider, however, is that natural genetic variability exists between individuals in any and every population with respect to pharmaceutically-important proteins, including the protein targets of candidate drugs, the enzymes that metabolize these drugs and the proteins whose activity is modulated by such drug targets. Subtle alteration(s) in the primary nucleotide sequence of a gene encoding a pharmaceutically-important protein may be manifested as significant variation in expression, structure and/or function of the protein. Such alterations may explain the relatively high degree of uncertainty inherent in the treatment of individuals with a drug whose design is based upon a single representative example of the target or enzyme(s) involved in metabolizing the drug. For example, it is well-established that some drugs frequently have lower efficacy in some individuals than others, which means such individuals and their physicians must weigh the possible benefit of a larger dosage against a greater risk of side effects. Also, there is significant variation in how well people metabolize drugs and other exogenous chemicals, resulting in substantial interindividual variation in the toxicity and/or efficacy of such exogenous substances (Evans et al., 1999, Science 286:487-491). This variability in efficacy or toxicity of a drug in genetically-diverse patients makes many drugs ineffective or even dangerous in certain groups of the population, leading to the failure of such drugs in clinical trials or their early withdrawal from the market even though they could be highly beneficial for other groups in the population. This problem significantly increases the time and cost of drug discovery and development, which is a matter of great public concern.

It is well-recognized by pharmaceutical scientists that considering the impact of the genetic variability of pharmaceutically-important proteins in the early phases of drug discovery and development is likely to reduce the failure rate of candidate and approved drugs (Marshall A 1997 Nature Biotech 15:1249-52; Kleyn P W et al. 1998 Science 281: 1820-21; Kola I 1999 Curr Opin Biotech 10:589-92; Hill A V S et al. 1999 in Evolution in Health and Disease Stearns S S (Ed.) Oxford University Press, New York, pp 62-76; Meyer U. A. 1999 in Evolution in Health and Disease Stearns S S (Ed.) Oxford University Press, New York, pp 41-49; Kalow W et al. 1999 Clin. Pharm. Therap. 66:445-7; Marshall, E 1999 Science 284:406-7; Judson R et al. 2000 Pharmacogenomics 1:1-12; Roses A D 2000 Nature 405:857-65). However, in practice this has been difficult to do, in large part because of the time and cost required for discovering the amount of genetic variation that exists in the population (Chakravarti A 1998 Nature Genet 19:216-7; Wang DG et al 1998 Science 280:1077-82; Chakravarti A 1999 Nat Genet 21:56-60 (suppl); Stephens J C 1999 Mol. Diagnosis 4:309-317; Kwok P Y and Gu S 1999 Mol. Med. Today 5:538-43; Davidson S 2000 Nature Biotech 18:1134-5).

The standard for measuring genetic variation among individuals is the haplotype, which is the ordered combination of polymorphisms in the sequence of each form of a gene that exists in the population. Because haplotypes represent the variation across each form of a gene, they provide a more accurate and reliable measurement of genetic variation than individual polymorphisms. For example, while specific variations in gene sequences have been associated with a particular phenotype such as disease susceptibility (Roses A D supra; Ulbrecht M et al. 2000 Am J Respir Crit Care Med 161: 469-74) and drug response (Wolfe C R et al. 2000 BMJ 320:987-90; Dahl B S 1997 Acta Psychiatr Scand 96 (Suppl 391): 14-21), in many other cases an individual polymorphism may be found in a variety of genomic backgrounds, i.e., different haplotypes, and therefore shows no definitive coupling between the polymorphism and the causative site for the phenotype (Clark A G et al. 1998 Am J Hum Genet 63:595-612; Ulbrecht M et al. 2000 supra; Drysdale et al. 2000 PNAS 97:10483-10488). Thus, there is an unmet need in the pharmaceutical industry for information on what haplotypes exist in the population for pharmaceutically-important genes. Such haplotype information would be useful in improving the efficiency and output of several steps in the drug discovery and development process, including target validation, identifying lead compounds, and early phase clinical trials (Marshall et al., supra).

One pharmaceutically-important gene for the treatment of obesity, diabetes, immunological disorders and other diseases associated with defects in body mass and thermoregulation is the uncoupling protein 2 (mitochondrial, proton carrier) (UCP2) gene or its encoded product. UCP2 is one of several nuclear DNA-encoded uncoupling proteins found in the mitochondria. Uncoupling proteins are transporters in the mitochondrial membrane that are involved in dissipating the proton electrochemical gradient, thereby releasing stored energy as heat. (Fleury et al., Nat. Genet 1997; 15:269-272). UCP2 has been shown to be up-regulated in white fat in response to fat feeding, which supports its role in energy dissipation.

Studies have suggested that UCP2 may be responsible for mitochondrial proton leaking, which contributes to the basal metabolic rate (Millet et al., J Clin Invest 1997; 100:2665-2670). Proton leaking decreases with increasing body mass in mammals, suggesting that differences in proton leaks may explain the differences in standard metabolic rate between mammals of different mass (Porter and Brand, Nature 1993 April 15;362(6421):628-30). Since UCP1, which is expressed specifically in brown adipose tissue and plays an important role in thermogenesis, is unlikely to play a major role in the regulation of energy expenditure in adults due to the small amount of brown adipose tissue present, UCP2 has been suggested as the candidate protein for the regulation of proton leaking (Millet et al. supra).

Neel (Am. J Hum. Genet. 1962; 14: 353-362) introduced the idea that a “thrifty” gene, which made some individuals highly energy efficient and some prone to obesity, may be responsible for diabetes and obesity. For example, Pima Indians presently living in the southwestern desert of Arizona are highly susceptible to type II diabetes and obesity (Abstract; Ravussin and Bogardus, Infusionstherapie 1990 April; 17(2):108-12). Studies have shown that at any given body weight and body composition, there is quite a large variability in the resting metabolic rate, which has been shown to be a familial trait. Thus, UCP2 may be a key gene in the control of metabolic efficiency and alterations in its function or expression could determine the “thrifty” phenotype.

UCP2 is also expressed in spleen, lung, and isolated macrophages suggesting it may play a role in immunity or inflammatory responsiveness. In a recent study using UCP2 deficient mice (UCP2−/−), Arsenijevic et al. (Nat. Genet 2000; 26:435-439) showed that UCP2−/− macrophages produced more reactive oxygen species (ROS) than wild-type macrophages. ROS are produced by stimulated macrophages and have potent toxoplasmacidal effects. Evidence suggests that modulating proton leakage through the inner mitochondrial membrane may regulate ROS production. Therefore, UCP2 may be an important modulator of immunological responses and manipulation of its expression or activity may serve as a novel therapeutic strategy for eradicating infectious agents.

The UCP2 gene is located on chromosome 11q13, whose homologous region on the mouse is tightly linked to the “tubby” mutation, which causes maturity-onset obesity, insulin resistance, retinal degeneration, and neurosensory hearing loss. In addition, Arsenijevic et al. (supra) reported that their chromosomal mapping is co-incident with quantitative trait loci for obesity from at least three independent mouse models, one congenic strain, and human insulin dependent diabetes locus-4. Thus, UCP2 may also play a role in obesity. Thus, UCP2 has a unique role in energy balance, body weight regulation, thermoregulation and responses to inflammatory stimuli.

The uncoupling protein 2 (mitochondrial, proton carrier) contains 6 exons that encode a 309 amino acid protein. A reference sequence for the UCP2 gene is shown in the contiguous lines of FIG. 1, which is a genomic sequence based on Genaissance Reference No. 7914833 (SEQ ID NO: 1). Reference sequences for the coding sequence (GenBank Accession No. NM_{—}003355.1) and protein are shown in FIGS. 2 (SEQ ID NO: 2) and 3 (SEQ ID NO: 3), respectively.

One polymorphism in the UCP2 identified by the Applicants herein has been previously reported: a polymorphism of cytosine or thymine at a position corresponding to nucleotide position 5600 in FIG. 1 results in a substitution of valine for alanine at a position corresponding to amino acid position 55 of the UCP2 protein in FIG. 3 (Argyropoulos et al., Diabetes 1998; 47:685-687). Thymine/thymine homozygotes for this polymorphic site have been reported to have enhanced metabolic activity and lower fat oxidation than individuals having other genotypes (Astrup et al., Int J Obes. Relat Metab Disord. 1999; 23:1030-1034). Also, African-Americans having a thymine/thymine nucleotide pair at this position are 1.9 times more likely to be diabetic than their cytosine/thymine or cytosine/cytosine counterparts (Garvey et al., PCT Publication WO 99/48905, Rieusset et al., Biochem Biophys. Res. Commun. 1999; 265:265-271). The cytosine/cytosine pair at nucleotide position 5600 in FIG. 1 has been associated with diabetes, a larger body mass index (BMI), increased blood pressure and increased HDL cholesterol (Garvey et al., supra). Caucasian cytosine/cytosine homozygotes are 2.4 times more likely to be diabetic than their cytosine/thymine or thymine/thymine counterparts (Garvey et al., supra). Further, both diabetic and non-diabetic Chinese women with the cytosine/cytosine nucleotide pair at a position corresponding to nucleotide position 5600 in FIG. 1 have decreased femoral subcutaneous adipose (English Translation of Abstract; Zheng et al., Zhonghua Yi. Xue. Yi. Chuan Xue. Za Zhi. 2000; 17:97-100) while Caucasian and African-American women with the cytosine/cytosine nucleotide pair have a higher BMI than females with the thymine/thymine nucleotide pair (Garvey et al., supra). Also, Caucasian and African-American women with the cytosine/cytosine nucleotide pair at this position have higher blood pressure and higher mean HDL cholesterol, respectively, as compared to women with other genotypes (Garvey et al., supra). Finally Pima Indians heterozygous for cytosine and thymine at a position corresponding to nucleotide position 5600 in FIG. 1 have a higher metabolic rate during sleep than Pima Indians with other genotypes (Walder et al., Hum Mol. Genet 1998; 7:1431-1435). This polymorphism was also reported by Vrolijk in PCT Publication WO 99/37812. This application discloses use of this polymorphism in diagnosing diseases such as obesity, non-insulin dependent diabetes mellitus, atherosclerosis, hyperinsulinemia, chronic inflammation, diseases related to thermogenic response, apoptosis and cachexia. However, the application does not specify which nucleotide at this polymorphic site is associated with any of these diseases.

Because of the potential for variation in the UCP2 gene to affect the expression and function of the encoded protein, it would be useful to know whether additional polymorphisms exist in the UCP2 gene, as well as how such polymorphisms are combined in different copies of the gene. Such information could be applied for studying the biological function of UCP2 as well as in identifying drugs targeting this protein for the treatment of disorders related to its abnormal expression or function.

### SUMMARY OF THE INVENTION

Accordingly, the inventors herein have discovered 22 novel polymorphic sites in the UCP2 gene. These polymorphic sites (PS) correspond to the following nucleotide positions in FIG. 1: 1283 (PS1), 1714 (PS2), 2051 (PS3), 2124 (PS4), 2287 (PS5), 2408 (PS6), 4768 (PS7), 4785 (PS8), 4813 (PS9), 4882 (PS10), 4976 (PS11), 5820 (PS13), 6536 (PS14), 6607 (PS15), 6617 (PS16), 6872 (PS17), 6966 (PS18), 7036 (PS19), 7086 (PS20), 8100 (PS21), 8221 (PS22) and 8677 (PS23). The polymorphisms at these sites are cytosine or guanine at PS1, cytosine or thymine at PS2, thymine or cytosine at PS3, cytosine or thymine at PS4, cytosine or guanine at PS5, adenine or guanine at PS6, adenine or guanine at PS7, guanine or adenine at PS8, thymine or cytosine at PS9, adenine or cytosine at PS10, thymine or adenine at PS11, thymine or guanine at PS13, thymine or adenine at PS14, guanine or adenine at PS15, cytosine or thymine at PS16, cytosine or guanine at PS17, guanine or adenine at PS18, cytosine or thymine at PS19, adenine or guanine at PS20, cytosine or thymine at PS21, guanine or adenine at PS22 and thymine or adenine at PS23. In addition, the inventors have determined the identity of the alleles at these sites, as well as at the previously identified site at nucleotide position 5600 (PS12), in a human reference population of 79 unrelated individuals self-identified as belonging to one of four major population groups: African descent, Asian, Caucasian and Hispanic/Latino. From this information, the inventors deduced a set of haplotypes and haplotype pairs for PS1-PS23 in the UCP2 gene, which are shown below in Tables 4 and 3, respectively. Each of these UCP2 haplotypes constitutes a code, or genetic marker, that defines the variant nucleotides that exist in the human population at this set of polymorphic sites in the UCP2 gene. Thus each UCP2 haplotype also represents a naturally-occurring isoform (also referred to herein as an “isogene”) of the UCP2 gene. The frequency of each haplotype and haplotype pair within the total reference population and within each of the four major population groups included in the reference population was also determined.

Thus, in one embodiment, the invention provides a method, composition and kit for genotyping the UCP2 gene in an individual. The genotyping method comprises identifying the nucleotide pair that is present at one or more polymorphic sites selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23 in both copies of the UCP2 gene from the individual. In some embodiments, the genotyping method may also comprise identifying the nucleotide pair that is present at PS12. A genotyping composition of the invention comprises an oligonucleotide probe or primer which is designed to specifically hybridize to a target region containing, or adjacent to, one of these UCP2 polymorphic sites. In one embodiment, a genotyping kit of the invention comprises a set of oligonucleotides designed to genotype each of these novel UCP2 polymorphic sites. In a preferred embodiment, the genotyping kit comprises a set of oligonucleotides designed to genotype each of PS1-PS23. The genotyping method, composition, and kit are useful in determining whether an individual has one of the haplotypes in Table 4 below or has one of the haplotype pairs in Table 3 below.

The invention also provides a method for haplotyping the UCP2 gene in an individual. In one embodiment, the haplotyping method comprises determining, for one copy of the UCP2 gene, the identity of the nucleotide at one or more polymorphic sites selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23. In another embodiment, the haplotyping method comprises determining whether one copy of the individual's UCP2 gene is defined by one of the UCP2 haplotypes shown in Table 4, below, or a sub-haplotype thereof. In a preferred embodiment, the haplotyping method comprises determining whether both copies of the individual's UCP2 gene are defined by one of the UCP2 haplotype pairs shown in Table 3 below, or a sub-haplotype pair thereof. Establishing the UCP2 haplotype or haplotype pair of an individual is useful for improving the efficiency and reliability of several steps in the discovery and development of drugs for treating diseases associated with UCP2 activity, e.g., obesity, diabetes, immunological disorders and other diseases associated with defects in body mass and thermoregulation.

For example, the haplotyping method can be used by the pharmaceutical research scientist to validate UCP2 as a candidate target for treating a specific condition or disease predicted to be associated with UCP2 activity. Determining for a particular population the frequency of one or more of the individual UCP2 haplotypes or haplotype pairs described herein will facilitate a decision on whether to pursue UCP2 as a target for treating the specific disease of interest. In particular, if variable UCP2 activity is associated with the disease, then one or more UCP2 haplotypes or haplotype pairs will be found at a higher frequency in disease cohorts than in appropriately genetically matched controls. Conversely, if each of the observed UCP2 haplotypes are of similar frequencies in the disease and control groups, then it may be inferred that variable UCP2 activity has little, if any, involvement with that disease. In either case, the pharmaceutical research scientist can, without a priori knowledge as to the phenotypic effect of any UCP2 haplotype or haplotype pair, apply the information derived from detecting UCP2 haplotypes in an individual to decide whether modulating UCP2 activity would be useful in treating the disease.

The claimed invention is also useful in screening for compounds targeting UCP2 to treat a specific condition or disease predicted to be associated with UCP2 activity. For example, detecting which of the UCP2 haplotypes or haplotype pairs disclosed herein are present in individual members of a population with the specific disease of interest enables the pharmaceutical scientist to screen for a compound(s) that displays the highest desired agonist or antagonist activity for each of the UCP2 isoforms present in the disease population, or for only the most frequent UCP2 isoforms present in the disease population. Thus, without requiring any a priori knowledge of the phenotypic effect of any particular UCP2 haplotype or haplotype pair, the claimed haplotyping method provides the scientist with a tool to identify lead compounds that are more likely to show efficacy in clinical trials.

Haplotyping the UCP2 gene in an individual is also useful in the design of clinical trials of candidate drugs for treating a specific condition or disease predicted to be associated with UCP2 activity. For example, instead of randomly assigning patients with the disease of interest to the treatment or control group as is typically done now, determining which of the UCP2 haplotype(s) disclosed herein are present in individual patients enables the pharmaceutical scientist to distribute UCP2 haplotypes and/or haplotype pairs evenly to treatment and control groups, thereby reducing the potential for bias in the results that could be introduced by a larger frequency of an UCP2 haplotype or haplotype pair that is associated with response to the drug being studied in the trial, even if this association was previously unknown. Thus, by practicing the claimed invention, the scientist can more confidently rely on the information learned from the trial, without first determining the phenotypic effect of any UCP2 haplotype or haplotype pair.

In another embodiment, the invention provides a method for identifying an association between a trait and an UCP2 genotype, haplotype, or haplotype pair for one or more of the novel polymorphic sites described herein. The method comprises comparing the frequency of the UCP2 genotype, haplotype, or haplotype pair in a population exhibiting the trait with the frequency of the UCP2 genotype or haplotype in a reference population. A different frequency of the UCP2 genotype, haplotype, or haplotype pair in the trait population than in the reference population indicates the trait is associated with the UCP2 genotype, haplotype, or haplotype pair. In preferred embodiments, the trait is susceptibility to a disease, severity of a disease, the staging of a disease or response to a drug. In a particularly preferred embodiment, the UCP2 haplotype is selected from the haplotypes shown in Table 4, or a sub-haplotype thereof. Such methods have applicability in developing diagnostic tests and therapeutic treatments for obesity, diabetes, immunological disorders and other diseases associated with defects in body mass and thermoregulation.

In yet another embodiment, the invention provides an isolated polynucleotide comprising a nucleotide sequence which is a polymorphic variant of a reference sequence for the UCP2 gene or a fragment thereof. The reference sequence comprises the contiguous sequences shown in FIG. 1 and the polymorphic variant comprises at least one polymorphism selected from the group consisting of guanine at PS1, thymine at PS2, cytosine at PS3, thymine at PS4, guanine at PS5, guanine at PS6, guanine at PS7, adenine at PS8, cytosine at PS9, cytosine at PS10, adenine at PS11, guanine at PS13, adenine at PS14, adenine at PS15, thymine at PS16, guanine at PS17, adenine at PS18, thymine at PS19, guanine at PS20, thymine at PS21, adenine at PS22 and adenine at PS23. In a preferred embodiment, the polymorphic variant comprises an additional polymorphism of thymine at PS12.

A particularly preferred polymorphic variant is an isogene of the UCP2 gene. An UCP2 isogene of the invention comprises cytosine or guanine at PS1, cytosine or thymine at PS2, thymine or cytosine at PS3, cytosine or thymine at PS4, cytosine or guanine at PS5, adenine or guanine at PS6, adenine or guanine at PS7, guanine or adenine at PS8, thymine or cytosine at PS9, adenine or cytosine at PS10, thymine or adenine at PS11, cytosine orthymine at PS12, thymine or guanine at PS13, thymine or adenine at PS14, guanine or adenine at PS15, cytosine or thymine at PS16, cytosine or guanine at PS17, guanine or adenine at PS18, cytosine or thymine at PS19, adenine or guanine at PS20, cytosine or thymine at PS21, guanine or adenine at PS22 and thymine or adenine at PS23. The invention also provides a collection of UCP2 isogenes, referred to herein as an UCP2 genome anthology.

In another embodiment, the invention provides a polynucleotide comprising a polymorphic variant of a reference sequence for an UCP2 cDNA or a fragment thereof. The reference sequence comprises SEQ ID NO:2 (FIG. 2) and the polymorphic cDNA comprises at least one polymorphism selected from the group consisting of adenine at a position corresponding to nucleotide 582 and thymine at a position corresponding to nucleotide 750. In a preferred embodiment, the polymorphic variant comprises an additional polymorphism of thymine at a position corresponding to nucleotide 164. A particularly preferred polymorphic cDNA variant is selected from the group consisting of A, B and C represented in Table 7.

Polynucleotides complementary to these UCP2 genomic and cDNA variants are also provided by the invention. It is believed that polymorphic variants of the UCP2 gene will be useful in studying the expression and function of UCP2, and in expressing UCP2 protein for use in screening for candidate drugs to treat diseases related to UCP2 activity.

In other embodiments, the invention provides a recombinant expression vector comprising one of the polymorphic genomic and cDNA variants operably linked to expression regulatory elements as well as a recombinant host cell transformed or transfected with the expression vector. The recombinant vector and host cell may be used to express UCP2 for protein structure analysis and drug binding studies.

The present invention also provides nonhuman transgenic animals comprising one or more of the UCP2 polymorphic genomic variants described herein and methods for producing such animals. The transgenic animals are useful for studying expression of the UCP2 isogenes in vivo, for in vivo screening and testing of drugs targeted against UCP2 protein, and for testing the efficacy of therapeutic agents and compounds for obesity, diabetes, immunological disorders and other diseases associated with defects in body mass and thermoregulation in a biological system.

The present invention also provides a computer system for storing and displaying polymorphism data determined for the UCP2 gene. The computer system comprises a computer processing unit; a display; and a database containing the polymorphism data. The polymorphism data includes one or more of the following: the polymorphisms, the genotypes, the haplotypes, and the haplotype pairs identified for the UCP2 gene in a reference population. In a preferred embodiment, the computer system is capable of producing a display showing UCP2 haplotypes organized according to their evolutionary relationships.

### BRIEF DESCRIPTION OF THE DRAWINGS

FIG. 1 illustrates a reference sequence for the UCP2 gene (Genaissance Reference No. 7914833; contiguous lines), with the start and stop positions of each region of coding sequence indicated with a bracket ([ or ]) and the numerical position below the sequence and the polymorphic site(s) and polymorphism(s) identified by Applicants in a reference population indicated by the variant nucleotide positioned below the polymorphic site in the sequence. SEQ ID NO:1 is equivalent to FIG. 1, with the two alternative allelic variants of each polymorphic site indicated by the appropriate nucleotide symbol (R=G or A, Y=T or C, M=A or C, K=G or T, S=G or C, and W=A or T; WIPO standard ST.25). SEQ ID NO:116 is a modified version of SEQ ID NO:1 that shows the context sequence of each polymorphic site, PS1-PS23, in a uniform format to facilitate electronic searching. For each polymorphic site, SEQ ID NO:116 contains a block of 60 bases of the nucleotide sequence encompassing the centrally-located polymorphic site at the 30^{th} position, followed by 60 bases of unspecified sequence to represent that each PS is separated by genomic sequence whose composition is defined elsewhere herein.

FIG. 2 illustrates a reference sequence for the UCP2 coding sequence (contiguous lines; SEQ ID NO:2), with the polymorphic site(s) and polymorphism(s) identified by Applicants in a reference population indicated by the variant nucleotide positioned below the polymorphic site in the sequence.

FIG. 3 illustrates a reference sequence for the UCP2 protein (contiguous lines; SEQ ID NO:3), with the variant amino acid(s) caused by the polymorphism(s) of FIG. 2 positioned below the polymorphic site in the sequence.

### DESCRIPTION OF THE PREFERRED EMBODIMENTS

The present invention is based on the discovery of novel variants of the UCP2 gene. As described in more detail below, the inventors herein discovered 16 isogenes of the UCP2 gene by characterizing the UCP2 gene found in genomic DNAs isolated from an Index Repository that contains immortalized cell lines from one chimpanzee and 93 human individuals. The human individuals included a reference population of 79 unrelated individuals self-identified as belonging to one of four major population groups: Caucasian (21 individuals), African descent (20 individuals), Asian (20 individuals), or Hispanic/Latino (18 individuals). To the extent possible, the members of this reference population were organized into population subgroups by their self-identified ethnogeographic origin as shown in Table 1 below. In addition, the Index Repository contains three unrelated indigenous American Indians (one from each of North, Central and South America), one three-generation Caucasian family (from the CEPH Utah cohort) and one two-generation African-American family.

TABLE 1
Population Groups in the Index Repository

| | | No. of |
|---|---|---|
| Population Group | Population Subgroup | Individuals |
| African descent | | 20 |
| | Sierra Leone | 1 |
| Asian | | 20 |
| | Burma | 1 |
| | China | 3 |
| | Japan | 6 |
| | Korea | 1 |
| | Philippines | 5 |
| | Vietnam | 4 |
| Caucasian | | 21 |
| | British Isles | 3 |
| | British Isles/Central | 4 |
| | British Isles/Eastern | 1 |
| | Central/Eastern | 1 |
| | Eastern | 3 |
| | Central/Mediterranean | 1 |
| | Mediterranean | 2 |
| | Scandinavian | 2 |
| Hispanic/Latino | | 18 |
| | Caribbean | 8 |
| | Caribbean (Spanish Descent) | 2 |
| | Central American (Spanish Descent) | 1 |
| | Mexican American | 4 |
| | South American (Spanish Descent) | 3 |

The UCP2 isogenes present in the human reference population are defined by haplotypes for 23 polymorphic sites in the UCP2 gene, 22 of which are believed to be novel. The UCP2 polymorphic sites identified by the inventors are referred to as PS1-PS23 to designate the order in which they are located in the gene (see Table 2 below), with the novel polymorphic sites referred to as PS1, PS2, PS3, PS4, PS5, PS6, PS7, PS8, PS9, PS10, PS11, PS13, PS14, PS15, PS16, PS17, PS18, PS19, PS20, PS21, PS22 and PS23. Using the genotypes identified in the Index Repository for PS1-PS23 and the methodology described in the Examples below, the inventors herein also determined the pair of haplotypes for the UCP2 gene present in individual human members of this repository. The human genotypes and haplotypes found in the repository for the UCP2 gene include those shown in Tables 3 and 4, respectively. The polymorphism and haplotype data disclosed herein are useful for validating whether UCP2 is a suitable target for drugs to treat obesity, diabetes, immunological disorders and other diseases associated with defects in body mass and thermoregulation, screening for such drugs and reducing bias in clinical trials of such drugs.

In the context of this disclosure, the following terms shall be defined as follows unless otherwise indicated:

Allele—A particular form of a genetic locus, distinguished from other forms by its particular nucleotide sequence or amino acid sequence, or one of the alternative polymorphisms found at a polymorphic site.

Candidate Gene—A gene which is hypothesized to be responsible for a disease, condition, or the response to a treatment, or to be correlated with one of these.

Gene—A segment of DNA that contains the coding sequence for a protein, wherein the segment may include promoters, exons, introns, and other untranslated regions that control expression.

Genotype—An unphased 5′ to 3′ sequence of nucleotide pair(s) found at one or more polymorphic sites in a locus on a pair of homologous chromosomes in an individual. As used herein, genotype includes a full-genotype and/or a sub-genotype as described below.

Full-Genotype—The unphased 5′ to 3′ sequence of nucleotide pairs found at all polymorphic sites examined herein in a locus on a pair of homologous chromosomes in a single individual.

Sub-Genotype—The unphased 5′ to 3′ sequence of nucleotides seen at a subset of the polymorphic sites examined herein in a locus on a pair of homologous chromosomes in a single individual.

Genotyping—A process for determining a genotype of an individual.

Haplotype—A 5′ to 3′ sequence of nucleotides found at one or more polymorphic sites in a locus on a single chromosome from a single individual. As used herein, haplotype includes a full-haplotype and/or a sub-haplotype as described below.

Full-Haplotype—The 5′ to 3′ sequence of nucleotides found at all polymorphic sites examined herein in a locus on a single chromosome from a single individual.

Sub-Haplotype—The 5′ to 3′ sequence of nucleotides seen at a subset of the polymorphic sites examined herein in a locus on a single chromosome from a single individual.

Haplotype Pair—The two haplotypes found for a locus in a single individual.

Haplotyping—A process for determining one or more haplotypes in an individual and includes use of family pedigrees, molecular techniques and/or statistical inference.

Haplotype Data—Information concerning one or more of the following for a specific gene: a listing of the haplotype pairs in each individual in a population; a listing of the different haplotypes in a population; frequency of each haplotype in that or other populations, and any known associations between one or more haplotypes and a trait.

Isoform—A particular form of a gene, mRNA, cDNA, coding sequence or the protein encoded thereby, distinguished from other forms by its particular sequence and/or structure.

Isogene—One of the isoforms (e.g., alleles) of a gene found in a population. An isogene (or allele) contains all of the polymorphisms present in the particular isoform of the gene.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Locus—A location on a chromosome or DNA molecule corresponding to a gene or a physical or phenotypic feature, where physical features include polymorphic sites.

Naturally-Occurring—A term used to designate that the object it is applied to, e.g., naturally-occurring polynucleotide or polypeptide, can be isolated from a source in nature and which has not been intentionally modified by man.

Nucleotide Pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic Site (PS)—A position on a chromosome or DNA molecule at which at least two alternative sequences are found in a population.

Polymorphic Variant (or Variant)—A gene, mRNA, cDNA, polypeptide, protein or peptide whose nucleotide or amino acid sequence varies from a reference sequence due to the presence of a polymorphism in the gene.

Polymorphism—The sequence variation observed in an individual at a polymorphic site. Polymorphisms include nucleotide substitutions, insertions, deletions and microsatellites and may, but need not, result in detectable differences in gene expression or protein function.

Polymorphism Data—Information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.

Polymorphism Database—A collection of polymorphism data arranged in a systematic or methodical way and capable of being individually accessed by electronic or other means.

Polynucleotide—A nucleic acid molecule comprised of single-stranded RNA or DNA or comprised of complementary, double-stranded DNA.

Population Group—A group of individuals sharing a common ethnogeographic origin.

Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—A human individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus is not known.

As discussed above, information on the identity of genotypes and haplotypes for the UCP2 gene of any particular individual as well as the frequency of such genotypes and haplotypes in any particular population of individuals is useful for a variety of drug discovery and development applications. Thus, the invention also provides compositions and methods for detecting the novel UCP2 polymorphisms, haplotypes and haplotype pairs identified herein.

The compositions comprise at least one oligonucleotide for detecting the variant nucleotide or nucleotide pair located at an UCP2 polymorphic site in one copy or two copies of the UCP2 gene. Such oligonucleotides are referred to herein as UCP2 haplotyping oligonucleotides or genotyping oligonucleotides, respectively, and collectively as UCP2 oligonucleotides. In one embodiment, an UCP2 haplotyping or genotyping oligonucleotide is a probe or primer capable of hybridizing to a target region that contains, or that is located close to, one of the novel polymorphic sites described herein.

As used herein, the term “oligonucleotide” refers to a polynucleotide molecule having less than about 100 nucleotides. A preferred oligonucleotide of the invention is 10 to 35 nucleotides long. More preferably, the oligonucleotide is between 15 and 30, and most preferably, between 20 and 25 nucleotides in length. The exact length of the oligonucleotide will depend on many factors that are routinely considered and practiced by the skilled artisan. The oligonucleotide may be comprised of any phosphorylation state of ribonucleotides, deoxyribonucleotides, and acyclic nucleotide derivatives, and other functionally equivalent derivatives. Alternatively, oligonucleotides may have a phosphate-free backbone, which may be comprised of linkages such as carboxymethyl, acetamidate, carbamate, polyamide (peptide nucleic acid (PNA)) and the like (Varma, R. in Molecular Biology and Biotechnology, A Comprehensive Desk Reference, Ed. R. Meyers, VCH Publishers, Inc. (1995), pages 617-620). Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Haplotyping or genotyping oligonucleotides of the invention must be capable of specifically hybridizing to a target region of an UCP2 polynucleotide. Preferably, the target region is located in an UCP2 isogene. As used herein, specific hybridization means the oligonucleotide forms an anti-parallel double-stranded structure with the target region under certain hybridizing conditions, while failing to form such a structure when incubated with another region in the UCP2 polynucleotide or with a non-UCP2 polynucleotide under the same hybridizing conditions. Preferably, the oligonucleotide specifically hybridizes to the target region under conventional high stringency conditions. The skilled artisan can readily design and test oligonucleotide probes and primers suitable for detecting polymorphisms in the UCP2 gene using the polymorphism information provided herein in conjunction with the known sequence information for the UCP2 gene and routine techniques.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, by Sambrook J. et al., in Molecular Cloning, A Laboratory Manual, 2^{nd} Edition, Cold Spring Harbor Press, Cold Spring Harbor, N.Y. (1989) and by Haymes, B. D. et al. in Nucleic Acid Hybridization, A Practical Approach, IRL Press, Washington, D.C. (1985). While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the probe or primer as long as the resulting probe or primer is still capable of specifically hybridizing to the target region.

Preferred haplotyping or genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruaño et al., 87 Proc. Natl. Acad. Sci. USA 6296-6300, 1990. Typically, an ASO will be perfectly complementary to one allele while containing a single mismatch for another allele.

Allele-specific oligonucleotides of the invention include ASO probes and ASO primers. ASO probes which usually provide good discrimination between different alleles are those in which a central position of the oligonucleotide probe aligns with the polymorphic site in the target region (e.g., approximately the 7^{th} or 8^{th} position in a 15 mer, the 8^{th} or 9^{th} position in a 16 mer, and the 10^{th} or 11^{th} position in a 20 mer). An ASO primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. ASO probes and primers hybridizing to either the coding or noncoding strand are contemplated by the invention. ASO probes and primers listed below use the appropriate nucleotide symbol (R=G or A, Y=T or C, M=A or C, K=G or T, S=G or C, and W=A or T; WIPO standard ST.25) at the position of the polymorphic site to represent that the ASO contains either of the two alternative allelic variants observed at that polymorphic site.

A preferred ASO probe for detecting UCP2 gene polymorphisms comprises a nucleotide sequence, listed 5′ to 3′, selected from the group consisting of:

| | |
|---|---|
| AACTGCTSAGCTCAA and its complement, | (SEQ ID NO:4) |
| CTCTGTCYCATCGTG and its complement, | (SEQ ID NO:5) |
| TTAGGTGYTTCGTCT and its complement, | (SEQ ID NO:6) |
| TGAAGAAYGGGACAC and its complement, | (SEQ ID NO:7) |
| CCCCACCSCCGACAG and its complement, | (SEQ ID NO:8) |
| AGGAGAARACTGAGG and its complement, | (SEQ ID NO:9) |
| TAAGCCTRTGGGTCT and its complement, | (SEQ ID NO:10) |
| GCCTGTTRGGTCTTA and its complement, | (SEQ ID NO:11) |
| TTTTCTCYACTTCTG and its complement, | (SEQ ID NO:12) |
| TCTGCATMCAGCAGA and its complement, | (SEQ ID NO:13) |
| GGAGCCCWTCATGAA and its complement, | (SEQ ID NO:l4) |
| TTCTCAGKGATGATT and its complement, | (SEQ ID NO:l5) |
| GATTCTAWTCCCAAA and its complement, | (SEQ ID NO:16) |
| AGTGCAARCCCGCTG and its complement, | (SEQ ID NO:17) |
| CGCTGGCYACTGACC and its complement, | (SEQ ID NO:18) |
| TTTCCTCSTCCCCGA and its complement, | (SEQ ID NO:19) |
| CTGAGCTRGTGACCT and its complement, | (SEQ ID NO:20) |
| AGGTAGAYGGTGCTG and its complement, | (SEQ ID NO:21) |
| GGGGTCTRGCTGACA and its complement, | (SEQ ID NO:22) |
| GCCAGTAYAGTAGCG and its complement, | (SEQ ID NO:23) |
| GTCTATTRTGGGTGG and its complement, | (SEQ ID NO:24) |
| and | |
| GATCACCWCTGGCTT and its complement. | (SEQ ID NO:25) |

A preferred ASO primer for detecting UCP2 gene polymorphisms comprises a nucleotide sequence, listed 5′ to 3′, selected from the group consisting of:

| | |
|---|---|
| GCCTCGAACTGCTSA; | (SEQ ID NO:26) |
| GATTGCTTGAGCTSA; | (SEQ ID NO:27) |
| GAGAGTCTCTGTCYC; | (SEQ ID NO:28) |
| GGGGGTCACGATGRG; | (SEQ ID NO:29) |
| TGACCATTAGGTGYT; | (SEQ ID NO:30) |
| GGTGGGAGACGAARC; | (SEQ ID NO:31) |
| CAGCTTTGAAGAAYG; | (SEQ ID NO:32) |
| CTAAAGGTGTCCCRT; | (SEQ ID NO:33) |
| TACCATCCCCACCSC; | (SEQ ID NO:34) |
| ACTTCACTGTCGGSG; | (SEQ ID NO:35) |
| TTATAAAGGAGAARA; | (SEQ ID NO:36) |
| CGTGGGCCTCAGTYT; | (SEQ ID NO:37) |
| AGAACATAAGCCTRT; | (SEQ ID NO:38) |
| AGGCACAGACCCAYA; | (SEQ ID NO:39) |
| GTCTGTGCCTGTTRG; | (SEQ ID NO:40) |
| CCAGACTAAGACCYA; | (SEQ ID NO:41) |
| TGAAACTTTTCTCYA; | (SEQ ID NO:42) |
| AGCTGACAGAAGTRG; | (SEQ ID NO:43) |
| GAAATCTCTGCATMC; | (SEQ ID NO:44) |
| CCTTTGTCTGCTGKA; | (SEQ ID NO:45) |
| AGGGAGGGAGCCCWT; | (SEQ ID NO:46) |
| CAATACTTCATGAWG; | (SEQ ID NO:47) |
| CCTTTTTTCTCAGKG; | (SEQ ID NO:48) |
| AAGATCAATCATCMC; | (SEQ ID NO:49) |
| CTTCTGGATTCTAWT; | (SEQ ID NO:50) |
| CCTGCTTTTGGGAWT; | (SEQ ID NO:51) |
| AAAATGAGTGCAARC; | (SEQ ID NO:52) |
| AGTGGCCAGCGGGYT; | (SEQ ID NO:53) |
| CAAGCCCGCTGGCYA; | (SEQ ID NO:54) |
| CCATGGGGTCACTRG; | (SEQ ID NO:55) |
| TCCCCTTTTCCTCST; | (SEQ ID NO:56) |
| AGAGTATCGGGGASG; | (SEQ ID NO:57) |
| ACTGTGCTGAGCTRG; | (SEQ ID NO:58) |
| GGTCATAGGTCACYA; | (SEQ ID NO:59) |
| GTCATGAGGTAGAYG; | (SEQ ID NO:GO) |
| GAGACCCAGCACCRT; | (SEQ ID NO:61) |
| GGTGCGGGGGTCTRG; | (SEQ ID NO:62) |
| TTCTGGTGTCAGCYA; | (SEQ ID NO:63) |
| CCCTGGGCCAGTAYA; | (SEQ ID NO:64) |
| GGCCAGCGCTACTRT; | (SEQ ID NO:65) |
| ATGCATGTCTATTRT; | (SEQ ID NO:66) |
| TCTCTCCCACCCAYA; | (SEQ ID NO:67) |
| TGACCTGATCACCWC and | (SEQ ID NO:68) |
| GAGACAAAGCCAGWG. | (SEQ ID NO:69) |

Other oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

A particularly preferred oligonucleotide primer for detecting UCP2 gene polymorphisms by primer extension terminates in a nucleotide sequence, listed 5′ to 3′, selected from the group consisting of:

| | |
|---|---|
| TCGAACTGCT; | (SEQ ID NO:70) |
| TGCTTGAGCT; | (SEQ ID NO:71) |
| AGTCTCTGTC; | (SEQ ID NO:72) |
| GGTCACGATG; | (SEQ ID NO:73) |
| CCATTAGGTG; | (SEQ ID NO:74) |
| GGGAGACGAA; | (SEQ ID NO:75) |
| CTTTGAAGAA; | (SEQ ID NO:76) |
| AAGGTGTCCC; | (SEQ ID NO:77) |
| CATCCCCACC; | (SEQ ID NO:78) |
| TCACTGTCGG; | (SEQ ID NO:79) |
| TAAAGGAGAA; | (SEQ ID NO:80) |
| GGGCCTCAGT; | (SEQ ID NO:81) |
| ACATAAGCCT; | (SEQ ID NO:82) |
| CACAGACCCA; | (SEQ ID NO:83) |
| TGTGCCTGTT; | (SEQ ID NO:84) |
| GACTAAGACC; | (SEQ ID NO:85) |
| AACTTTTCTC; | (SEQ ID NO:86) |
| TGACAGAAGT; | (SEQ ID NO:87) |
| ATCTCTGCAT; | (SEQ ID NO:88) |
| TTGTCTGCTG; | (SEQ ID NO:89) |
| GAGGGAGCCC; | (SEQ ID NO:90) |
| TACTTCATGA; | (SEQ ID NO:91) |
| TTTTTCTCAG; | (SEQ ID NO:92) |
| ATCAATCATC; | (SEQ ID NO:93) |
| CTGGATTCTA; | (SEQ ID NO:94) |
| GCTTTTGGGA; | (SEQ ID NO:95) |
| ATGAGTGCAA; | (SEQ ID NO:96) |
| GGCCAGCGGG; | (SEQ ID NO:97) |
| GCCCGCTGGC; | (SEQ ID NO:98) |
| TGGGGTCAGT; | (SEQ ID NO:99) |
| CCTTTTCCTC; | (SEQ ID NO:100) |
| GTATCGGGGA; | (SEQ ID NO:101) |
| GTGCTGAGCT; | (SEQ ID NO:102) |
| CATAGGTCAC; | (SEQ ID NO:103) |
| ATGAGGTAGA; | (SEQ ID NO:104) |
| AGCCAGCACC; | (SEQ ID NO:105) |
| GCGGGGGTCT; | (SEQ ID NO:106) |
| TGGTGTCAGC; | (SEQ ID NO:107) |
| TGGGCCAGTA; | (SEQ ID NO:108) |
| CAGCGCTACT; | (SEQ ID NO:109) |
| CATGTCTATT; | (SEQ ID NO:110) |
| CTCCCACCCA; | (SEQ ID NO:111) |
| CCTGATCACC; and | (SEQ ID NO:112) |
| ACAAAGCCAG. | (SEQ ID NO:113) |

In some embodiments, a composition contains two or more differently labeled UCP2 oligonucleotides for simultaneously probing the identity of nucleotides or nucleotide pairs at two or more polymorphic sites. It is also contemplated that primer compositions may contain two or more sets of allele-specific primer pairs to allow simultaneous targeting and amplification of two or more regions containing a polymorphic site.

UCP2 oligonucleotides of the invention may also be immobilized on or synthesized on a solid surface such as a microchip, bead, or glass slide (see, e.g., WO 98/20020 and WO 98/20019). Such immobilized oligonucleotides may be used in a variety of polymorphism detection assays, including but not limited to probe hybridization and polymerase extension assays. Immobilized UCP2 oligonucleotides of the invention may comprise an ordered array of oligonucleotides designed to rapidly screen a DNA sample for polymorphisms in multiple genes at the same time.

In another embodiment, the invention provides a kit comprising at least two UCP2 oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

The above described oligonucleotide compositions and kits are useful in methods for genotyping and/or haplotyping the UCP2 gene in an individual. As used herein, the terms “UCP2 genotype” and “UCP2 haplotype” mean the genotype or haplotype contains the nucleotide pair or nucleotide, respectively, that is present at one or more of the novel polymorphic sites described herein and may optionally also include the nucleotide pair or nucleotide present at one or more additional polymorphic sites in the UCP2 gene. The additional polymorphic sites may be currently known polymorphic sites or sites that are subsequently discovered.

One embodiment of a genotyping method of the invention involves examining both copies of the individual's UCP2 gene, or a fragment thereof, to identify the nucleotide pair at one or more polymorphic sites selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23 in the two copies to assign an UCP2 genotype to the individual. In some embodiments, “examining a gene” may include examining one or more of: DNA containing the gene, mRNA transcripts thereof, or cDNA copies thereof. As will be readily understood by the skilled artisan, the two “copies” of a gene, mRNA or cDNA (or fragment of such UCP2 molecules) in an individual may be the same allele or may be different alleles. In a preferred embodiment of the method for assigning an UCP2 genotype, the identity of the nucleotide pair at PS12 is also determined. In another embodiment, a genotyping method of the invention comprises determining the identity of the nucleotide pair at each of PS1-PS23.

One method of examining both copies of the individual's UCP2 gene is by isolating from the individual a nucleic acid sample comprising the two copies of the UCP2 gene, mRNA transcripts thereof or cDNA copies thereof, or a fragment of any of the foregoing, that are present in the individual. Typically, the nucleic acid sample is isolated from a biological sample taken from the individual, such as a blood sample or tissue sample. Suitable tissue samples include whole blood, semen, saliva, tears, urine, fecal material, sweat, buccal, skin and hair. The nucleic acid sample may be comprised of genomic DNA, mRNA, or cDNA and, in the latter two cases, the biological sample must be obtained from a tissue in which the UCP2 gene is expressed. Furthermore it will be understood by the skilled artisan that mRNA or cDNA preparations would not be used to detect polymorphisms located in introns or in 5′ and 3′ untranslated regions if not present in the mRNA or cDNA. If an UCP2 gene fragment is isolated, it must contain the polymorphic site(s) to be genotyped.

One embodiment of a haplotyping method of the invention comprises examining one copy of the individual's UCP2 gene, or a fragment thereof, to identify the nucleotide at one or more polymorphic sites selected from the group consisting of PS1, PS2, PS3, PS4, PS5, PS6, PS7, PS8, PS9, PS10, PS1, PS13, PS14, PS15, PS16, PS17, PS18, PS19, PS20, PS21, PS22 and PS23 in that copy to assign an UCP2 haplotype to the individual. In another embodiment of the haplotyping method, the identity of the nucleotide at PS12is also determined. In a preferred embodiment, the nucleotide at each of PS1-PS23 is identified. In a particularly preferred embodiment, the UCP2 haplotype assigned to the individual is selected from the group consisting of the UCP2 haplotypes shown in Table 4.

In some embodiments, “examining a gene” may include examining one or more of: DNA containing the gene, mRNA transcripts thereof, or cDNA copies thereof. One method of examining one copy of the individual's UCP2 gene is by isolating from the individual a nucleic acid sample containing only one of the two copies of the UCP2 gene, mRNA or cDNA, or a fragment of such UCP2 molecules, that is present in the individual and determining in that copy the identity of the nucleotide at one or more polymorphic sites selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23 to assign an UCP2 haplotype to the individual. In some embodiments, the UCP2 haplotype is assigned to the individual by also identifying the nucleotide at PS12. In a particularly preferred embodiment, the nucleotide at each of PS1-PS23 is identified.

In another embodiment, the haplotyping method comprises determining whether an individual has one or more of the UCP2 haplotypes shown in Table 4. This can be accomplished by identifying the phased sequence of nucleotides present at PS1-PS23 for at least one copy of the individual's UCP2 gene and assigning to that copy an UCP2 haplotype that is consistent with the phased sequence, wherein the UCP2 haplotype is selected from the group consisting of the UCP2 haplotypes shown in Table 4 and wherein each of the UCP2 haplotypes in Table 4 comprises a sequence of polymorphisms whose positions and alleles are set forth in the table. This identifying step does not necessarily require that each of PS1-PS23 be directly examined. Typically only a subset of PS1-PS23 will need to be directly examined to assign to an individual one or more of the haplotypes shown in Table 4. This is because for at least one polymorphic site in a gene, the allele present is frequently in strong linkage disequilibrium with the allele at one or more other polymorphic sites in that gene (Drysdale, C M et al. 2000 PNAS 97:10483-10488; Rieder M J et al. 1999 Nature Genetics 22:59-62). Two nucleotide alleles are said to be in linkage disequilibrium if the presence of a particular allele at one polymorphic site predicts the presence of the other allele at a second polymorphic site (Stevens, J C, Mol. Diag. 4: 309-17, 1999). Techniques for determining whether alleles at any two polymorphic sites are in linkage disequilibrium are well-known in the art (Weir B. S. 1996 Genetic Data Analysis II, Sinauer Associates, Inc. Publishers, Sunderland, Mass.). In addition, Johnson et al. (2001 Nature Genetics 29: 233-237) presented one possible method for selection of subsets of polymorphic sites suitable for identifying known haplotypes.

In another embodiment of a haplotyping method of the invention, an UCP2 haplotype pair is determined for an individual by identifying the phased sequence of nucleotides at one or more polymorphic sites selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23 in each copy of the UCP2 gene that is present in the individual. In a particularly preferred embodiment, the haplotyping method comprises identifying the phased sequence of nucleotides at each of PS1-PS23 in each copy of the UCP2 gene.

In another embodiment, the haplotyping method comprises determining whether an individual has one of the UCP2 haplotype pairs shown in Table 3. One way to accomplish this is to identify the phased sequence of nucleotides at PS1-PS23 for each copy of the individual's UCP2 gene and assigning to the individual an UCP2 haplotype pair that is consistent with each of the phased sequences, wherein the UCP2 haplotype pair is selected from the group consisting of the UCP2 haplotype pairs shown in Table 3. As described above, the identifying step does not necessarily require that each of PS1-PS23 be directly examined. As a result of linkage disequilibrium, typically only a subset of PS1-PS23 will need to be directly examined to assign to an individual a haplotype pair shown in Table 3.

The nucleic acid used in the above haplotyping methods of the invention may be isolated using any method capable of separating the two copies of the UCP2 gene or fragment such as one of the methods described above for preparing UCP2 isogenes, with targeted in vivo cloning being the preferred approach. As will be readily appreciated by those skilled in the art, any individual clone will typically only provide haplotype information on one of the two UCP2 gene copies present in an individual. If haplotype information is desired for the individual's other copy, additional UCP2 clones will usually need to be examined. Typically, at least five clones should be examined to have more than a 90% probability of haplotyping both copies of the UCP2 gene in an individual. In some cases, however, once the haplotype for one UCP2 allele is directly determined, the haplotype for the other allele may be inferred if the individual has a known genotype for the polymorphic sites of interest or if the haplotype frequency or haplotype pair frequency for the individual's population group is known.

When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the UCP2 gene, or a fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

The target region(s) may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-193, 1991; WO90/01069), and oligonucleotide ligation assay (OLA) (Landegren et al., Science 241:1077-1080, 1988). Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., Proc. Natl. Acad. Sci. USA 89:392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the UCP2 gene of an individual may also be determined by hybridization of a nucleic acid sample containing one or both copies of the gene, mRNA, cDNA or fragment(s) thereof, to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nucl. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruaño et al., Nucl. Acids Res. 17:8392, 1989; Ruaño et al., Nucl. Acids Res. 19, 6877-6882, 1991; WO 93/22456; Turki et al., J. Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

In addition, the identity of the allele(s) present at any of the novel polymorphic sites described herein may be indirectly determined by haplotyping or genotyping the allele(s) at another polymorphic site that is in linkage disequilibrium with the allele at the polymorphic site of interest. Polymorphic sites with alleles in linkage disequilibrium with the alleles of presently disclosed polymorphic sites may be located in regions of the gene or in other genomic regions not examined herein. Detection of the allele(s) present at a polymorphic site in linkage disequilibrium with the allele(s) of novel polymorphic sites described herein may be performed by, but is not limited to, any of the above-mentioned methods for detecting the identity of the allele at a polymorphic site.

In another aspect of the invention, an individual's UCP2 haplotype pair is predicted from its UCP2 genotype using information on haplotype pairs known to exist in a reference population. In its broadest embodiment, the haplotyping prediction method comprises identifying an UCP2 genotype for the individual at two or more UCP2 polymorphic sites described herein, accessing data containing UCP2 haplotype pairs identified in a reference population, and assigning a haplotype pair to the individual that is consistent with the individual's UCP2 genotype. In one embodiment, the reference haplotype pairs include the UCP2 haplotype pairs shown in Table 3. The UCP2 haplotype pair can be assigned by comparing the individual's genotype with the genotypes corresponding to the haplotype pairs known to exist in the general population or in a specific population group, and determining which haplotype pair is consistent with the genotype of the individual. In some embodiments, the comparing step may be performed by visual inspection (for example, by consulting Table 3). When the genotype of the individual is consistent with more than one haplotype pair, frequency data (such as that presented in Table 6) may be used to determine which of these haplotype pairs is most likely to be present in the individual. This determination may also be performed in some embodiments by visual inspection, for example by consulting Table 6. If a particular UCP2 haplotype pair consistent with the genotype of the individual is more frequent in the reference population than others consistent with the genotype, then that haplotype pair with the highest frequency is the most likely to be present in the individual. In other embodiments, the comparison may be made by a computer-implemented algorithm with the genotype of the individual and the reference haplotype data stored in computer-readable formats. For example, as described in WO 01/80156, one computer-implemented algorithm to perform this comparison entails enumerating all possible haplotype pairs which are consistent with the genotype, accessing data containing UCP2 haplotype pair frequency data determined in a reference population to determine a probability that the individual has a possible haplotype pair, and analyzing the determined probabilities to assign a haplotype pair to the individual.

Generally, the reference population should be composed of randomly-selected individuals representing the major ethnogeographic groups of the world. A preferred reference population for use in the methods of the present invention comprises an approximately equal number of individuals from Caucasian, African-descent, Asian and Hispanic-Latino population groups with the minimum number of each group being chosen based on how rare a haplotype one wants to be guaranteed to see. For example, if one wants to have a q% chance of not missing a haplotype that exists in the population at a p% frequency of occurring in the reference population, the number of individuals (n) who must be sampled is given by 2 n=log(1−q)/log(1−p) where p and q are expressed as fractions. A preferred reference population allows the detection of any haplotype whose frequency is at least 10% with about 99% certainty and comprises about 20 unrelated individuals from each of the four population groups named above. A particularly preferred reference population includes a 3-generation family representing one or more of the four population groups to serve as controls for checking quality of haplotyping procedures.

In a preferred embodiment, the haplotype frequency data for each ethnogeographic group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3^{rd} Ed., 1997) postulates that the frequency of finding the haplotype pair H₁/H₂ is equal to p_{H-W}(H₁/H₂)=2 p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), single molecule dilution (SMD), or allele-specific long-range PCR (Michalotos-Beloin et al., Nucleic Acids Res. 24:4841-4843, 1996).

In one embodiment of this method for predicting an UCP2 haplotype pair for an individual, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. Alternatively, the haplotype pair in an individual may be predicted from the individual's genotype for that gene using reported methods (e.g., Clark et al. 1990 Mol Bio Evol 7:111-22 or WO 01/80156) or through a commercial haplotyping service such as offered by Genaissance Pharmaceuticals, Inc. (New Haven, Conn.). In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

The invention also provides a method for determining the frequency of an UCP2 genotype, haplotype, or haplotype pair in a population. The method comprises, for each member of the population, determining the genotype, haplotype or the haplotype pair for the novel UCP2 polymorphic sites described herein, and calculating the frequency any particular genotype, haplotype, or haplotype pair is found in the population. The population may be e.g., a reference population, a family population, a same gender population, a population group, or a trait population (e.g., a group of individuals exhibiting a trait of interest such as a medical condition or response to a therapeutic treatment).

In one embodiment of the invention, UCP2 haplotype frequencies in a trait population having a medical condition and a control population lacking the medical condition are used in a method of validating the UCP2 protein as a candidate target for treating a medical condition predicted to be associated with UCP2 activity. The method comprises comparing the frequency of each UCP2 haplotype shown in Table 4 in the trait population and in a control population and making a decision whether to pursue UCP2 as a target. It will be understood by the skilled artisan that the composition of the control population will be dependent upon the specific study and may be a reference population or it may be an appropriately matched population with regards to age, gender, and clinical symptoms for example. If at least one UCP2 haplotype is present at a frequency in the trait population that is different from the frequency in the control population at a statistically significant level, a decision to pursue the UCP2 protein as a target should be made. However, if the frequencies of each of the UCP2 haplotypes are not statistically significantly different between the trait and control populations, a decision not to pursue the UCP2 protein as a target is made. The statistically significant level of difference in the frequency may be defined by the skilled artisan practicing the method using any conventional or operationally convenient means known to one skilled in the art, taking into consideration that this level should help the artisan to make a rational decision about pursuing UCP2 protein as a target. Any UCP2 haplotype not present in a population is considered to have a frequency of zero. In some embodiments, each of the trait and control populations may be comprised of different ethnogeographic origins, including but not limited to Caucasian, Hispanic Latino, African American, and Asian, while in other embodiments, the trait and control populations may be comprised of just one ethnogeographic origin.

In another embodiment of the invention, frequency data for UCP2 haplotypes are determined in a population having a condition or disease predicted to be associated with UCP2 activity and used in a method for screening for compounds targeting the UCP2 protein to treat such condition or disease. In some embodiments, frequency data are determined in the population of interest for the UCP2 haplotypes shown in Table 4. The frequency data for this population may be obtained by genotyping or haplotyping each individual in the population using one or more of the methods described above. The haplotypes for this population may be determined directly or, alternatively, by a predictive genotype to haplotype approach as described above. In another embodiment, the frequency data for this population are obtained by accessing previously determined frequency data, which may be in written or electronic form. For example, the frequency data may be present in a database that is accessible by a computer. The UCP2 isoforms corresponding to UCP2 haplotypes occurring at a frequency greater than or equal to a desired frequency in this population are then used in screening for a compound, or compounds, that displays a desired agonist (enhancer) or antagonist (inhibitor) activity for each UCP2 isoform. The desired frequency for the haplotypes might be chosen to be the frequency of the most frequent haplotype, greater than or less than some cut-off value, such as 10% in the population, or the desired frequency might be determined by ranking the haplotypes by frequency and then choosing the frquency of the third most frequent haplotype as the cut-off value. Other methods for choosing a desired frequency are possible, such as choosing a frequency based on the desired market size for treatment with the compound. The desired level of agonist or antagonist level displayed in the screening process could be chosen to be greater than or equal to a cut-off value, such as activity levels in the top 10% of values determined. Embodiments may employ cell-free or cell-based screening assays known in the art. The compounds used in the screening assays may be from chemical compound libraries, peptide libraries and the like. The UCP2 isoforms used in the screening assays may be free in solution, affixed to a solid support, or expressed in an appropriate cell line.

In some of the above embodiments, the condition or disease associated with UCP2 activity may be obesity, diabetes, immunological disorders or other diseases associated with defects in body mass and thermoregulation.

In another aspect of the invention, frequency data for UCP2 genotypes, haplotypes, and/or haplotype pairs are determined in a reference population and used in a method for identifying an association between a trait and an UCP2 genotype, haplotype, or haplotype pair. The trait may be any detectable phenotype, including but not limited to susceptibility to a disease or response to a treatment. In one embodiment, the method involves obtaining data on the frequency of the genotype(s), haplotype(s), or haplotype pair(s) of interest in a reference population as well as in a population exhibiting the trait. Frequency data for one or both of the reference and trait populations may be obtained by genotyping or haplotyping each individual in the populations using one or more of the methods described above. The haplotypes for the trait population may be determined directly or, alternatively, by a predictive genotype to haplotype approach as described above. In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s), haplotype(s), or haplotype pair(s) of interest in the reference and trait populations are compared. In a preferred embodiment, the frequencies of all genotypes, haplotypes, and/or haplotype pairs observed in the populations are compared. If the frequency of a particular UCP2 genotype, haplotype, or haplotype pair is different in the trait population than in the reference population to a statistically significant degree, then the trait is predicted to be associated with that UCP2 genotype, haplotype or haplotype pair. Preferably, the UCP2 genotype, haplotype, or haplotype pair being compared in the trait and reference populations is selected from the genotypes and haplotypes shown in Tables 3 and 4, or from sub-genotypes and sub-haplotypes derived from these genotypes and haplotypes. Sub-genotypes useful in the invention preferably do not include sub-genotypes solely for PS12.

In a preferred embodiment of the method, the trait of interest is a clinical response exhibited by a patient to some therapeutic treatment, for example, response to a drug targeting UCP2 or response to a therapeutic treatment for a medical condition. As used herein, “medical condition” includes but is not limited to any condition or disease manifested as one or more physical and/or psychological symptoms for which treatment is desirable, and includes previously and newly identified diseases and other disorders. As used herein the term “clinical response” means any or all of the following: a quantitative measure of the response, no response, and/or adverse response (i.e., side effects).

In order to deduce a correlation between clinical response to a treatment and an UCP2 genotype, haplotype, or haplotype pair, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials. As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the UCP2 gene for each individual in the trial population is genotyped and/or haplotyped, which may be done before or after administering the treatment.

After both the clinical and polymorphism data have been obtained, correlations between individual response and UCP2 genotype or haplotype content are created. Correlations may be produced in several ways. In one method, individuals are grouped by their UCP2 genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the UCP2 gene give the most significant contribution to the differences in phenotype. One regression model useful in the invention is described in WO 01/01218, entitled “Methods for Obtaining and Using Haplotype Data”.

A second method for finding correlations between UCP2 haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2^{nd} Edition (McGraw-Hill, New York, 1991, Ch. 18), standard gradient descent methods (Press et al., supra, Ch. 10), or other global or local optimization approaches (see discussion in Judson, supra) could also be used. Preferably, the correlation is found using a genetic algorithm approach as described in WO 01/01218.

Correlations may also be analyzed using analysis of variation (ANOVA) techniques to determine how much of the variation in the clinical data is explained by different subsets of the polymorphic sites in the UCP2 gene. As described in WO 01/01218, ANOVA is used to test hypotheses about whether a response variable is caused by or correlated with one or more traits or variables that can be measured (Fisher and vanBelle, supra, Ch. 10).

From the analyses described above, a mathematical model may be readily constructed by the skilled artisan that predicts clinical response as a function of UCP2 genotype or haplotype content. Preferably, the model is validated in one or more follow-up clinical trials designed to test the model.

The identification of an association between a clinical response and a genotype or haplotype (or haplotype pair) for the UCP2 gene may be the basis for designing a diagnostic method to determine those individuals who will or will not respond to the treatment, or alternatively, will respond at a lower level and thus may require more treatment, i.e., a greater dose of a drug. The diagnostic method will detect the presence in an individual of the genotype, haplotype or haplotype pair that is associated with the clinical response and may take one of several forms: for example, a direct DNA test (i.e., genotyping or haplotyping one or more of the polymorphic sites in the UCP2 gene), a serological test, or a physical exam measurement. The only requirement is that there be a good correlation between the diagnostic test results and the underlying UCP2 genotype or haplotype that is in turn correlated with the clinical response. In a preferred embodiment, this diagnostic method uses the predictive haplotyping method described above.

Another embodiment of the invention comprises a method for reducing the potential for bias in a clinical trial of a candidate drug for treating a disease or condition predicted to be associated with UCP2 activity. Haplotyping one or both copies of the UCP2 gene in those individuals participating in the trial will allow the pharmaceutical scientist conducting the clinical trial to assign each individual from the trial one of the UCP2 haplotypes or haplotype pairs shown in Tables 4 and 3, respectively, or an UCP2 sub-haplotype or sub-haplotype pair thereof. In one embodiment, the haplotypes may be determined directly, or alternatively, by a predictive genotype to haplotype approach as decribed above. In another embodiment, this can be accomplished by haplotyping individuals participating in a clinical trial by identifying, for example, in one or both copies of the individual's UCP2 gene, the phased sequence of nucleotides present at each of PS1-PS23. Determining the UCP2 haplotype or haplotype pair present in individuals participating in the clinical trial enables the pharmaceutical scientist to assign individuals possessing a specific haplotype or haplotype pair evenly to treatment and control groups. Typical clinical trials conducted may include, but are not limited to, Phase I, II, and III clinical trials. If the trial is measuring response to a drug for treating a disease or condition predicted to be associated with UCP2 activity, each individual in the trial may produce a specific response to the candidate drug based upon the individual's haplotype or haplotype pair. To control for these differing drug responses in the trial and to reduce the potential for bias in the results that could be introduced by a larger frequency of an UCP2 haplotype or haplotype pair in any particular treatment or control group due to random group assignment, each treatment and control group are assigned an even distribution (or equal numbers) of individuals having a particular UCP2 haplotype or haplotype pair. To practice this method of the invention to reduce the potential for bias in a clinical trial, the pharmaceutical scientist requires no a priori knowledge of any effect an UCP2 haplotype or haplotype pair may have on the results of the trial. Diseases or conditions predicted to be associated with UCP2 activity include, e.g., obesity, diabetes, immunological disorders and other diseases associated with defects in body mass and thermoregulation.

In another embodiment, the invention provides an isolated polynucleotide comprising a polymorphic variant of the UCP2 gene or a fragment of the gene which contains at least one of the novel polymorphic sites described herein. The nucleotide sequence of a variant UCP2 gene is identical to the reference genomic sequence for those portions of the gene examined, as described in the Examples below, except that it comprises a different nucleotide at one or more of the novel polymorphic sites PS1, PS2, PS3, PS4, PS5, PS6, PS7, PS8, PS9, PS10, PS11, PS13, PS14, PS15, PS16, PS17, PS18, PS19, PS20, PS21, PS22 and PS23, and may also comprise an additional polymorphism of thymine at PS12. Similarly, the nucleotide sequence of a variant fragment of the UCP2 gene is identical to the corresponding portion of the reference sequence except for having a different nucleotide at one or more of the novel polymorphic sites described herein. Thus, the invention specifically does not include polynucleotides comprising a nucleotide sequence identical to the reference sequence of the UCP2 gene, which is defined by haplotype 2, (or other reported UCP2 sequences) or to portions of the reference sequence (or other reported UCP2 sequences), except for the haplotyping and genotyping oligonucleotides described above.

The location of a polymorphism in a variant UCP2 gene or fragment is preferably identified by aligning its sequence against SEQ ID NO:1. The polymorphism is selected from the group consisting of guanine at PS1, thymine at PS2, cytosine at PS3, thymine at PS4, guanine at PS5, guanine at PS6, guanine at PS7, adenine at PS8, cytosine at PS9, cytosine at PS10, adenine at PS11, guanine at PS13, adenine at PS14, adenine at PS15, thymine at PS16, guanine at PS17, adenine at PS18, thymine at PS19, guanine at PS20, thymine at PS21, adenine at PS22 and adenine at PS23. In a preferred embodiment, the polymorphic variant comprises a naturally-occurring isogene of the UCP2 gene which is defined by any one of haplotypes 1 and 3-16 shown in Table 4 below.

Polymorphic variants of the invention may be prepared by isolating a clone containing the UCP2 gene from a human genomic library. The clone may be sequenced to determine the identity of the nucleotides at the novel polymorphic sites described herein. Any particular variant or fragment thereof, that is claimed herein could be prepared from this clone by performing in vitro mutagenesis using procedures well-known in the art. Any particular UCP2 variant or fragment thereof may also be prepared using synthetic or semi-synthetic methods known in the art.

UCP2 isogenes, or fragments thereof, may be isolated using any method that allows separation of the two “copies” of the UCP2 gene present in an individual, which, as readily understood by the skilled artisan, may be the same allele or different alleles. Separation methods include targeted in vivo cloning (TIVC) in yeast as described in WO 98/01573, U.S. Pat. No. 5,866,404, and U.S. Pat. No. 5,972,614. Another method, which is described in U.S. Pat. No. 5,972,614, uses an allele specific oligonucleotide in combination with primer extension and exonuclease degradation to generate hemizygous DNA targets. Yet other methods are SMD as described in Ruaño et al., Proc. Natl. Acad. Sci. 87:6296-6300, 1990; and allele specific PCR (Ruaño et al., 1989, supra; Ruaño et al., 1991, supra; Michalatos-Beloin et al., supra).

The invention also provides UCP2 genome anthologies, which are collections of at least two UCP2 isogenes found in a given population. The population may be any group of at least two individuals, including but not limited to a reference population, a population group, a family population, a clinical population, and a same gender population. An UCP2 genome anthology may comprise individual UCP2 isogenes stored in separate containers such as microtest tubes, separate wells of a microtitre plate and the like. Alternatively, two or more groups of the UCP2 isogenes in the anthology may be stored in separate containers. Individual isogenes or groups of such isogenes in a genome anthology may be stored in any convenient and stable form, including but not limited to in buffered solutions, as DNA precipitates, freeze-dried preparations and the like. A preferred UCP2 genome anthology of the invention comprises a set of isogenes defined by the haplotypes shown in Table 4 below.

An isolated polynucleotide containing a polymorphic variant nucleotide sequence of the invention may be operably linked to one or more expression regulatory elements in a recombinant expression vector capable of being propagated and expressing the encoded UCP2 protein in a prokaryotic or a eukaryotic host cell. Examples of expression regulatory elements which may be used include, but are not limited to, the lac system, operator and promoter regions of phage lambda, yeast promoters, and promoters derived from vaccinia virus, adenovirus, retroviruses, or SV40. Other regulatory elements include, but are not limited to, appropriate leader sequences, termination codons, polyadenylation signals, and other sequences required for the appropriate transcription and subsequent translation of the nucleic acid sequence in a given host cell. Of course, the correct combinations of expression regulatory elements will depend on the host system used. In addition, it is understood that the expression vector contains any additional elements necessary for its transfer to and subsequent replication in the host cell. Examples of such elements include, but are not limited to, origins of replication and selectable markers. Such expression vectors are commercially available or are readily constructed using methods known to those in the art (e.g., F. Ausubel et al., 1987, in “Current Protocols in Molecular Biology”, John Wiley and Sons, New York, N.Y.). Host cells which may be used to express the variant UCP2 sequences of the invention include, but are not limited to, eukaryotic and mammalian cells, such as animal, plant, insect and yeast cells, and prokaryotic cells, such as E. coli, or algal cells as known in the art. The recombinant expression vector may be introduced into the host cell using any method known to those in the art including, but not limited to, microinjection, electroporation, particle bombardment, transduction, and transfection using DEAE-dextran, lipofection, or calcium phosphate (see e.g., Sambrook et al. (1989) in “Molecular Cloning. A Laboratory Manual”, Cold Spring Harbor Press, Plainview, N.Y.). In a preferred aspect, eukaryotic expression vectors that function in eukaryotic cells, and preferably mammalian cells, are used. Non-limiting examples of such vectors include vaccinia virus vectors, adenovirus vectors, herpes virus vectors, and baculovirus transfer vectors. Preferred eukaryotic cell lines include COS cells, CHO cells, HeLa cells, NIH/3T3 cells, and embryonic stem cells (Thomson, J. A. et al., 1998 Science 282:1145-1147). Particularly preferred host cells are mammalian cells.

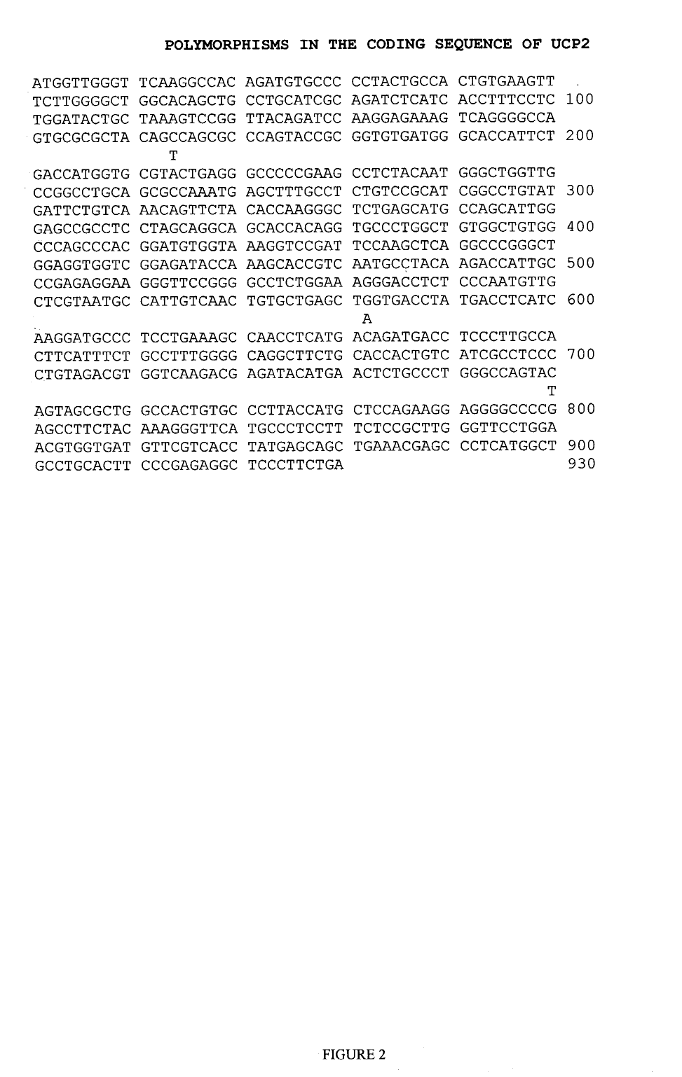

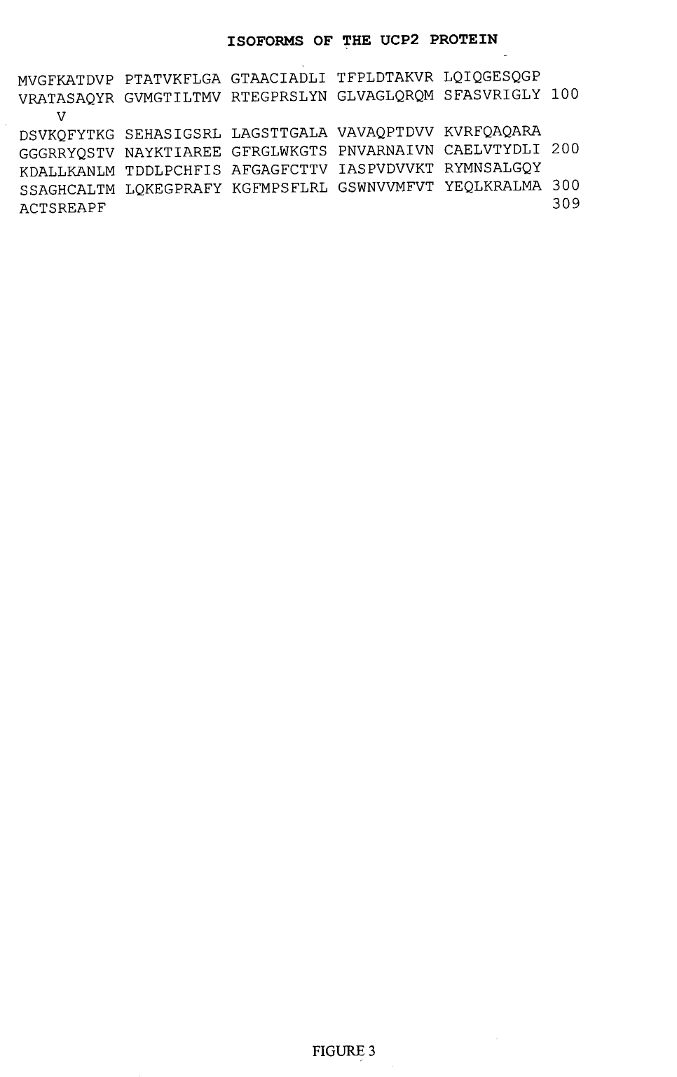

As will be readily recognized by the skilled artisan, expression of polymorphic variants of the UCP2 gene will produce UCP2 mRNAs varying from each other at any polymorphic site retained in the spliced and processed mRNA molecules. These mRNAs can be used for the preparation of an UCP2 cDNA comprising a nucleotide sequence which is a polymorphic variant of the UCP2 reference coding sequence shown in FIG. 2. Thus, the invention also provides UCP2 mRNAs and corresponding cDNAs which comprise a nucleotide sequence that is identical to SEQ ID NO:2 (FIG. 2) (or its corresponding RNA sequence) for those regions of SEQ ID NO:2 that correspond to the examined portions of the UCP2 gene (as described in the Examples below), except for having one or more polymorphisms selected from the group consisting of adenine at a position corresponding to nucleotide 582 and thymine at a position corresponding to nucleotide 750, and may also comprise an additional polymorphism of thymine at a position corresponding to nucleotide 164. A particularly preferred polymorphic cDNA variant is selected from the group consisting of A, B and C represented in Table 7. Fragments of these variant mRNAs and cDNAs are included in the scope of the invention, provided they contain one or more of the novel polymorphisms described herein. The invention specifically excludes polynucleotides identical to previously identified UCP2 mRNAs or cDNAs, and previously described fragments thereof. Polynucleotides comprising a variant UCP2 RNA or DNA sequence may be isolated from a biological sample using well-known molecular biological procedures or may be chemically synthesized.

As used herein, a polymorphic variant of an UCP2 gene fragment, mRNA fragment or cDNA fragment comprises at least one novel polymorphism identified herein and has a length of at least 10 nucleotides and may range up to the full length of the gene. Preferably, such fragments are between 100 and 3000 nucleotides in length, and more preferably between 100 and 2000 nucleotides in length, and most preferably between 100 and 500 nucleotides in length.

In describing the UCP2 polymorphic sites identified herein, reference is made to the sense strand of the gene for convenience. However, as recognized by the skilled artisan, nucleic acid molecules containing the UCP2 gene or cDNA may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides which are complementary to the sense strand of the UCP2 genomic, mRNA and cDNA variants described herein.

Polynucleotides comprising a polymorphic gene variant or fragment of the invention may be useful for therapeutic purposes. For example, where a patient could benefit from expression, or increased expression, of a particular UCP2 protein isoform, an expression vector encoding the isoform may be administered to the patient. The patient may be one who lacks the UCP2 isogene encoding that isoform or may already have at least one copy of that isogene.

In other situations, it may be desirable to decrease or block expression of a particular UCP2 isogene. Expression of an UCP2 isogene may be turned off by transforming a targeted organ, tissue or cell population with an expression vector that expresses high levels of untranslatable mRNA or antisense RNA for the isogene or fragment thereof. Alternatively, oligonucleotides directed against the regulatory regions (e.g., promoter, introns, enhancers, 3′ untranslated region) of the isogene may block transcription. Oligonucleotides targeting the transcription initiation site, e.g., between positions −10 and +10 from the start site are preferred. Similarly, inhibition of transcription can be achieved using oligonucleotides that base-pair with region(s) of the isogene DNA to form triplex DNA (see e.g., Gee et al. in Huber, B. E. and B. I. Carr, Molecular and Immunologic Approaches, Futura Publishing Co., Mt. Kisco, N.Y., 1994). Antisense oligonucleotides may also be designed to block translation of UCP2 mRNA transcribed from a particular isogene. It is also contemplated that ribozymes may be designed that can catalyze the specific cleavage of UCP2 mRNA transcribed from a particular isogene.

The untranslated mRNA, antisense RNA or antisense oligonucleotides may be delivered to a target cell or tissue by expression from a vector introduced into the cell or tissue in vivo or ex vivo. Alternatively, such molecules may be formulated as a pharmaceutical composition for administration to the patient. Oligoribonucleotides and/or oligodeoxynucleotides intended for use as antisense oligonucleotides may be modified to increase stability and half-life. Possible modifications include, but are not limited to phosphorothioate or 2′ O-methyl linkages, and the inclusion of nontraditional bases such as inosine and queosine, as well as acetyl-, methyl-, thio-, and similarly modified forms of adenine, cytosine, guanine, thymine, and uracil which are not as easily recognized by endogenous nucleases.

Effect(s) of the polymorphisms identified herein on expression of UCP2 may be investigated by various means known in the art, such as by in vitro translation of mRNA transcripts of the UCP2 gene, cDNA or fragment thereof, or by preparing recombinant cells and/or nonhuman recombinant organisms, preferably recombinant animals, containing a polymorphic variant of the UCP2 gene. As used herein, “expression” includes but is not limited to one or more of the following: transcription of the gene into precursor mRNA; splicing and other processing of the precursor mRNA to produce mature mRNA; mRNA stability; translation of the mature mRNA(s) into UCP2 protein(s) (including effects of polymorphisms on codon usage and tRNA availability); and glycosylation and/or other modifications of the translation product, if required for proper expression and function.

To prepare a recombinant cell of the invention, the desired UCP2 isogene, cDNA or coding sequence may be introduced into the cell in a vector such that the isogene, cDNA or coding sequence remains extrachromosomal. In such a situation, the gene will be expressed by the cell from the extrachromosomal location. In a preferred embodiment, the UCP2 isogene, cDNA or coding sequence is introduced into a cell in such a way that it recombines with the endogenous UCP2 gene present in the cell. Such recombination requires the occurrence of a double recombination event, thereby resulting in the desired UCP2 gene polymorphism. Vectors for the introduction of genes both for recombination and for extrachromosomal maintenance are known in the art, and any suitable vector or vector construct may be used in the invention. Methods such as electroporation, particle bombardment, calcium phosphate co-precipitation and viral transduction for introducing DNA into cells are known in the art; therefore, the choice of method may lie with the competence and preference of the skilled practitioner. Examples of cells into which the UCP2 isogene, cDNA or coding sequence may be introduced include, but are not limited to, continuous culture cells, such as COS, CHO, NIH/3T3, and primary or culture cells of the relevant tissue type, i.e., they express the UCP2 isogene, cDNA or coding sequence. Such recombinant cells can be used to compare the biological activities of the different protein variants.

Recombinant nonhuman organisms, i.e., transgenic animals, expressing a variant UCP2 gene, cDNA or coding sequence are prepared using standard procedures known in the art. Preferably, a construct comprising the variant gene, cDNA or coding sequence is introduced into a nonhuman animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes (or cDNA or coding sequence) of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells. Examples of animals into which the UCP2 isogene, cDNA or coding sequences may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W. H. Freeman and Company, New York, pages 254-272). Transgenic animals stably expressing a human UCP2 isogene, cDNA or coding sequence and producing the encoded human UCP2 protein can be used as biological models for studying diseases related to abnormal UCP2 expression and/or activity, and for screening and assaying various candidate drugs, compounds, and treatment regimens to reduce the symptoms or effects of these diseases.

An additional embodiment of the invention relates to pharmaceutical compositions for treating disorders affected by expression or function of a novel UCP2 isogene described herein. The pharmaceutical composition may comprise any of the following active ingredients: a polynucleotide comprising one of these novel UCP2 isogenes (or cDNAs or coding sequences); an antisense oligonucleotide directed against one of the novel UCP2 isogenes, a polynucleotide encoding such an antisense oligonucleotide, or another compound which inhibits expression of a novel UCP2 isogene described herein. Preferably, the composition contains the active ingredient in a therapeutically effective amount. By therapeutically effective amount is meant that one or more of the symptoms relating to disorders affected by expression or function of a novel UCP2 isogene is reduced and/or eliminated. The composition also comprises a pharmaceutically acceptable carrier, examples of which include, but are not limited to, saline, buffered saline, dextrose, and water. Those skilled in the art may employ a formulation most suitable for the active ingredient, whether it is a polynucleotide, oligonucleotide, protein, peptide or small molecule antagonist. The pharmaceutical composition may be administered alone or in combination with at least one other agent, such as a stabilizing compound. Administration of the pharmaceutical composition may be by any number of routes including, but not limited to oral, intravenous, intramuscular, intra-arterial, intramedullary, intrathecal, intraventricular, intradermal, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.).

For any composition, determination of the therapeutically effective dose of active ingredient and/or the appropriate route of administration is well within the capability of those skilled in the art. For example, the dose can be estimated initially either in cell culture assays or in animal models. The animal model may also be used to determine the appropriate concentration range and route of administration. Such information can then be used to determine useful doses and routes for administration in humans. The exact dosage will be determined by the practitioner, in light of factors relating to the patient requiring treatment, including but not limited to severity of the disease state, general health, age, weight and gender of the patient, diet, time and frequency of administration, other drugs being taken by the patient, and tolerance/response to the treatment.

Any or all analytical and mathematical operations involved in practicing the methods of the present invention may be implemented by a computer. In addition, the computer may execute a program that generates views (or screens) displayed on a display device and with which the user can interact to view and analyze large amounts of information relating to the UCP2 gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The UCP2 polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

The Examples herein are meant to exemplify the various aspects of carrying out the invention and are not intended to limit the scope of the invention in any way. The Examples do not include detailed descriptions for conventional methods employed, such as in the performance of genomic DNA isolation, PCR and sequencing procedures. Such methods are well-known to those skilled in the art and are described in numerous publications, for example, Sambrook, Fritsch, and Maniatis, “Molecular Cloning: A Laboratory Manual”, 2^{nd} Edition, Cold Spring Harbor Laboratory Press, USA, (1989).

### Example 1

This example illustrates examination of various regions of the UCP2 gene for polymorphic sites.

Amplification of Target Regions

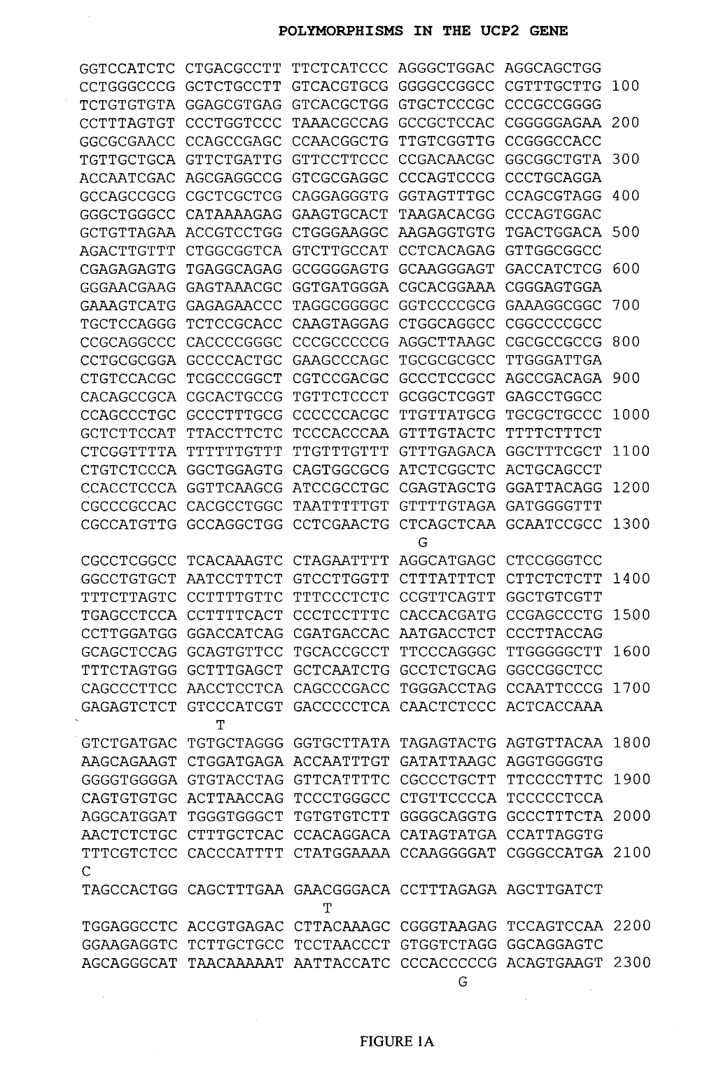

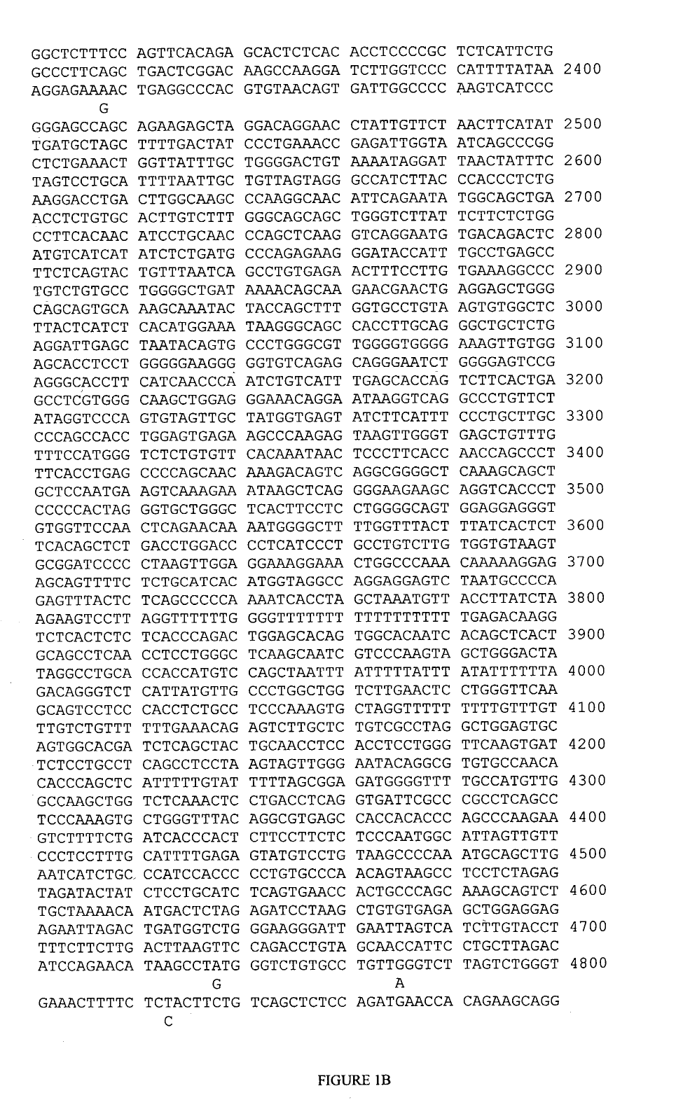

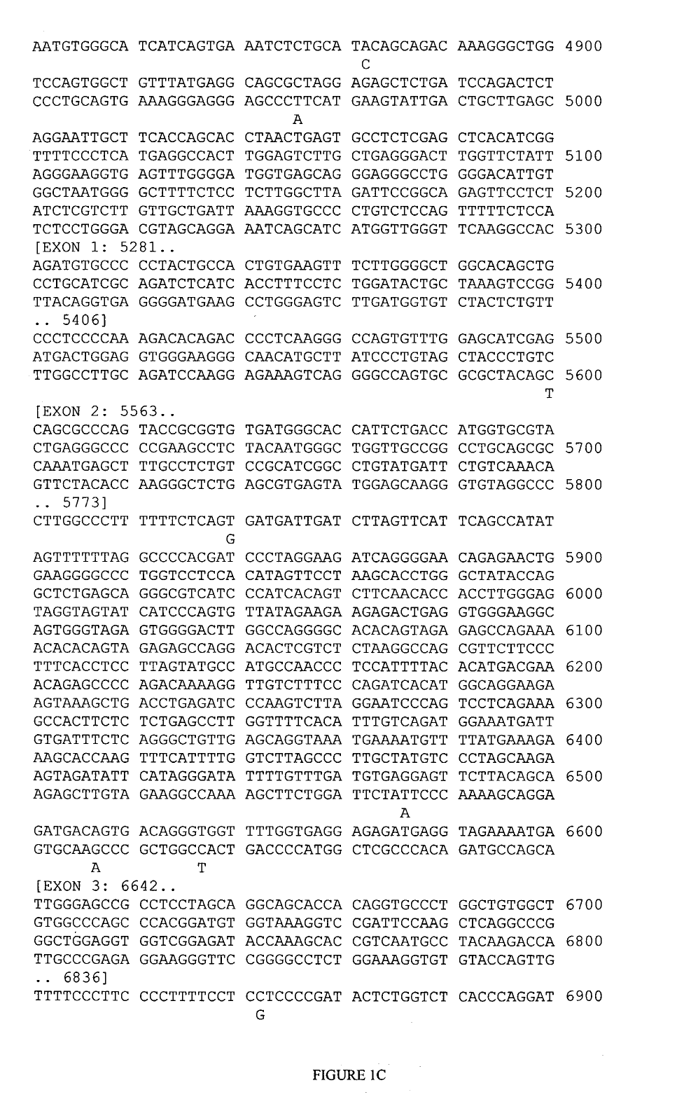

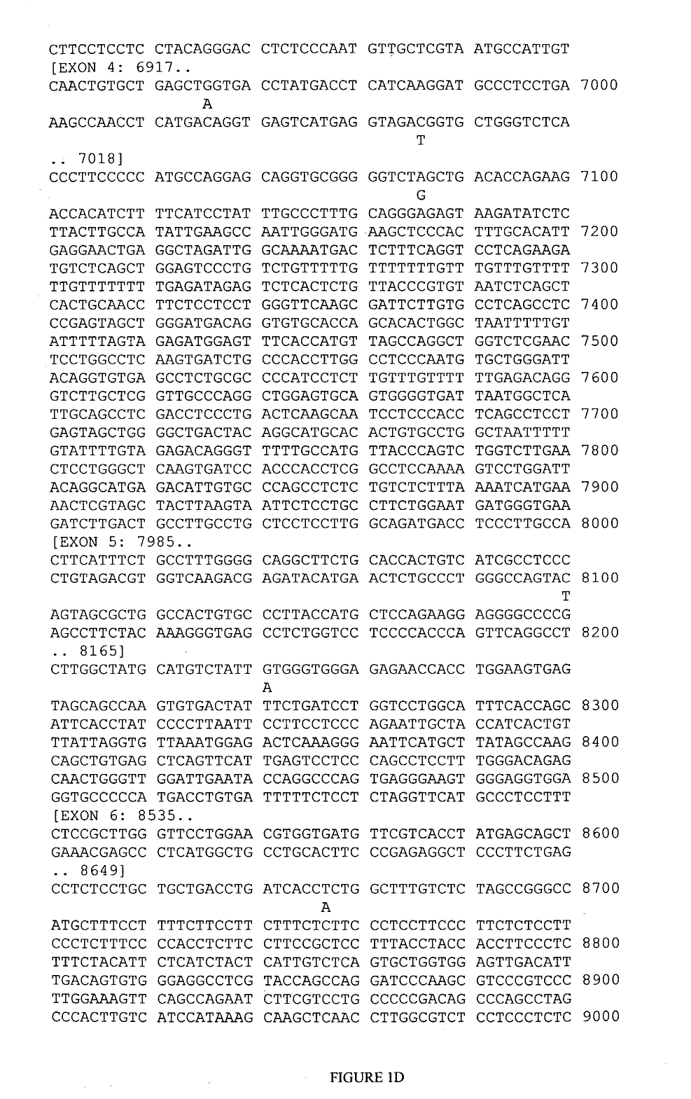

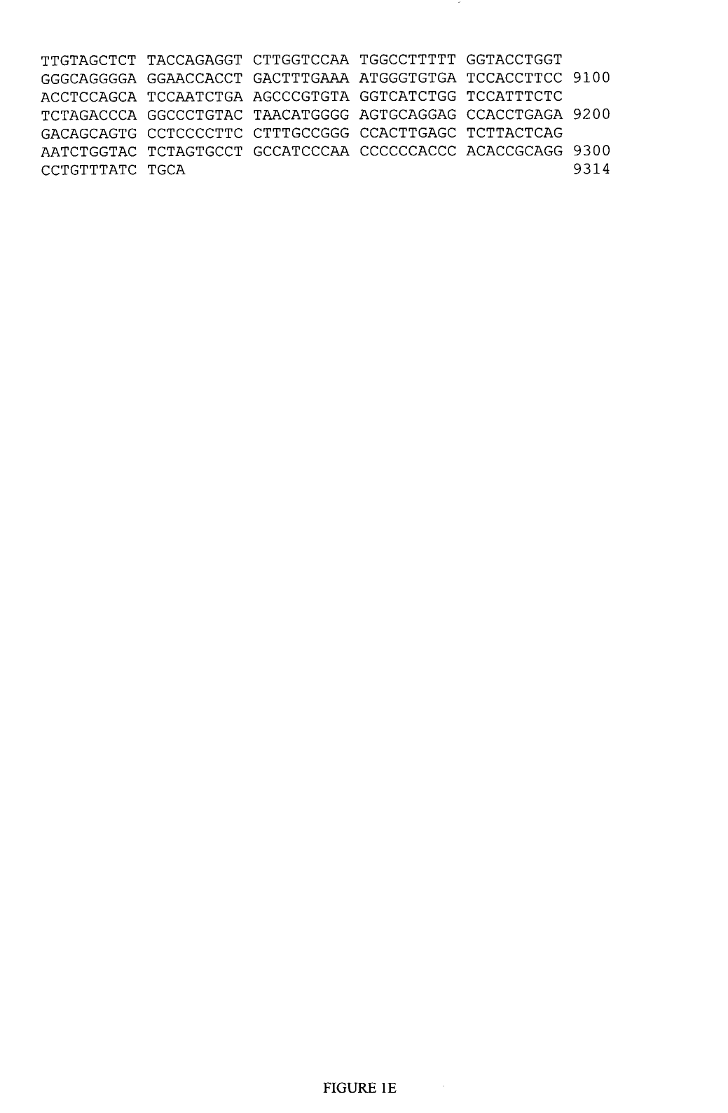

The following target regions of the UCP2 gene were amplified using ‘tailed’ PCR primers, each of which includes a universal sequence forming a noncomplementary ‘tail’ attached to the 5′ end of each unique sequence in the PCR primer pairs. The universal ‘tail’ sequence for the forward PCR primers comprises the sequence 5′-TGTAAAACGACGGCCAGT-3′ (SEQ ID NO:114) and the universal ‘tail’ sequence for the reverse PCR primers comprises the sequence 5′-AGGAAACAGCTATGACCAT-3′ (SEQ ID NO:115). The nucleotide positions of the first and last nucleotide of the forward and reverse primers for each region amplified are presented below and correspond to positions in SEQ ID NO:1 (FIG. 1).

PCR Primer Pairs

| | | | PCR |
|---|---|---|---|
| Fragment No. | Forward Primer | Reverse Primer | Product |
| Fragment 1 | 1000-1023 | complement of 1520-1501 | 521 nt |
| Fragment 2 | 1459-1481 | complement of 2063-2041 | 605 nt |
| Fragment 3 | 1875-1894 | complement of 2266-2245 | 392 nt |
| Fragment 4 | 2120-2143 | complement of 2531-2509 | 412 nt |
| Fragment 5 | 4393-4416 | complement of 4899-4878 | 507 nt |
| Fragment 6 | 4719-4741 | complement of 5236-5214 | 518 nt |
| Fragment 7 | 5399-5421 | complement of 5908-5887 | 510 nt |
| Fragment 8 | 6413-6434 | complement of 6997-6978 | 585 nt |
| Fragment 9 | 6767-6789 | complement of 7225-7203 | 459 nt |
| Fragment 10 | 7764-7786 | complement of 8311-8288 | 548 nt |
| Fragment 11 | 8367-8389 | complement of 8792-8770 | 426 nt |

These primer pairs were used in PCR reactions containing genomic DNA isolated from immortalized cell lines for each member of the Index Repository. The PCR reactions were carried out under the following conditions:

| | |
|---|---|
| Reaction volume = | 10 μl |
| 10 x Advantage 2 Polymerase reaction buffer (Clontech) = | 1 μl |
| 100 ng of human genomic DNA = | 1 μl |
| 10 mM dNTP = | 0.4 μl |
| Advantage 2 Polymerase enzyme mix (Clontech) = | 0.2 μl |
| Forward Primer (10 μM) = | 0.4 μl |
| Reverse Primer (10 μM) = | 0.4 μl |
| Water = | 6.6 μl |

Amplification profile:

| | | |
|---|---|---|
| 97° C. - 2 min. | | 1 cycle |
| 97° C. - 15 sec. | | |
| 70° C. - 45 sec. | {close oversize brace} | 10 cycles |
| 72° C. - 45 sec. | | |
| 97° C. - 15 sec. | | |
| 64° C. - 45 sec. | {close oversize brace} | 35 cycles |
| 72° C. - 45 sec. | | |

Sequencing of PCR Products

The PCR products were purified using a Whatman/Polyfiltronics 100 μl 384 well unifilter plate essentially according to the manufacturers protocol. The purified DNA was eluted in 50 μl of distilled water. Sequencing reactions were set up using Applied Biosystems Big Dye Terminator chemistry essentially according to the manufacturers protocol. The purified PCR products were sequenced in both directions using the appropriate universal ‘tail’ sequence as a primer. Reaction products were purified by isopropanol precipitation, and run on an Applied Biosystems 3700 DNA Analyzer.

Analysis of Sequences for Polymorphic Sites

Sequence information for a minimum of 80 humans was analyzed for the presence of polymorphisms using the Polyphred program (Nickerson et al., Nucleic Acids Res. 14:2745-2751, 1997). The presence of a polymorphism was confirmed on both strands. The polymorphisms and their locations in the UCP2 reference genomic sequence (SEQ ID NO:1) are listed in Table 2 below.

TABLE 2
Polymorphic Sites Identified in the UCP2 Gene

| Poly- | | | | | | |
|---|---|---|---|---|---|---|
| morphic | | Nucleo- | Refer- | | CDS | |
| Site | | tide | ence | Variant | Variant | AA |
| Number | Poly Id(a) | Position | Allele | Allele | Position | Variant |
| PS1 | 19739483 | 1283 | C | G | | |
| PS2 | 19710249 | 1714 | C | T | | |
| PS3 | 20295785 | 2051 | T | C | | |
| PS4 | 19711756 | 2124 | C | T | | |
| PS5 | 19711585 | 2287 | C | G | | |
| PS6 | 19755463 | 2408 | A | G | | |
| PS7 | 12396842 | 4768 | A | G | | |
| PS8 | 12396937 | 4785 | G | A | | |
| PS9 | 12397032 | 4813 | T | C | | |
| PS10 | 12397127 | 4882 | A | C | | |
| PS11 | 12397319 | 4976 | T | A | | |
| PS12(R) | 12435227 | 5600 | C | T | 164 | A55V |
| PS13 | 12394884 | 5820 | T | G | | |
| PS14 | 19704876 | 6536 | T | A | | |
| PS15 | 12392216 | 6607 | G | A | | |
| PS16 | 12392126 | 6617 | C | T | | |
| PS17 | 12468071 | 6872 | C | G | | |
| PS18 | 12391946 | 6966 | G | A | 582 | L194L |
| PS19 | 12468353 | 7036 | C | T | | |
| PS20 | 12468449 | 7086 | A | G | | |
| PS21 | 12398622 | 8100 | C | T | 750 | Y250Y |
| PS22 | 12398343 | 8221 | G | A | | |
| PS23 | 12387439 | 8677 | T | A | | |

(a)PolyId is a unique identifier assigned to each PS by Genaissance Pharmaceuticals, Inc.
(R)Reported previously.

### Example 2

This example illustrates analysis of the UCP2 polymorphisms identified in the Index Repository for human genotypes and haplotypes.

The different genotypes containing these polymorphisms that were observed in unrelated members of the reference population are shown in Table 3 below, with the haplotype pair indicating the combination of haplotypes determined for the individual using the haplotype derivation protocol described below. In Table 3, homozygous positions are indicated by one nucleotide and heterozygous positions are indicated by two nucleotides.

TABLE 3
Genotypes Observed for the UCP2 Gene

| Genotype | | | Polymorphic Sites | | | | | | | | | | | |
|---|---|---|---|---|---|---|---|---|---|---|---|---|---|---|
| Number | HAP Pair | | PS1 | PS2 | PS3 | PS4 | PS5 | PS6 | PS7 | PS8 | PS9 | PS10 | PS11 | PS12 |
| 1 | 3 | 3 | C | C | T | C | C | A | A | G | T | A | T | T |
| 2 | 3 | 4 | C | C | T | C | C | A | A | G | T | A | T | T |
| 3 | 3 | 12 | C/G | C | T | C | C | A | A | G | T | A | T | T/C |
| 4 | 3 | 13 | C/G | C | T | C | C | A | A | G | T | A | T | T/C |
| 5 | 3 | 14 | C/G | C | T | C | C | A | A | G | T | A | T | T |
| 6 | 3 | 16 | C/G | C/T | T | C/T | C | A | A | G | T | A | T | T/C |
| 7 | 11 | 1 | G/C | C | T | C | C | A | A | G/A | T | A | T | C/T |
| 8 | 11 | 2 | G/C | C | T | C | C | A | A | G | T | A | T | C |
| 9 | 11 | 3 | G/C | C | T | C | C | A | A | G | T | A | T | C/T |
| 10 | 11 | 4 | G/C | C | T | C | C | A | A | G | T | A | T | C/T |
| 11 | 11 | 5 | G/C | C | T | C | C | A | A/G | G | T/C | A/C | T | C/T |
| 12 | 11 | 6 | G/C | C | T | C | C | A/G | A | G | T | A | T | C/T |
| 13 | 11 | 7 | G/C | C | T | C | C/G | A | A | G | T | A | T | C |
| 14 | 11 | 8 | G | C | T/C | C | C | A | A | G | T | A | T/A | C |
| 15 | 11 | 9 | G | C | T | C | C | A | A | G | T | A | T | C |
| 16 | 11 | 10 | G | C | T | C | C | A | A | G | T | A | T | C |
| 17 | 11 | 11 | G | C | T | C | C | A | A | G | T | A | T | C |
| 18 | 11 | 12 | G | C | T | C | C | A | A | G | T | A | T | C |
| 19 | 11 | 15 | G | C/T | T | C | C/G | A | A | G | T | A | T | C |
| 20 | 11 | 16 | G | C/T | T | C/T | C | A | A | G | T | A | T | C |

| | |
|---|---|
| Genotype | Polymorphic Sites |

| Number | HAP Pair | | PS13 | PS14 | PS15 | PS16 | PS17 | PS18 | PS19 | PS20 | PS21 | PS22 | PS23 |
|---|---|---|---|---|---|---|---|---|---|---|---|---|---|
| 1 | 3 | 3 | T | T | G | C | C | G | C | A | C | G | T |
| 2 | 3 | 4 | T | T | G | C | C/G | G | C | A/G | C | G | T |
| 3 | 3 | 12 | T | T | G | C | C | G | C/T | A | C | G | T |
| 4 | 3 | 13 | T | T | G | C/T | C | G/A | C | A | C | G | T |
| 5 | 3 | 14 | T | T | G | C | C | G | C | A | C | G | T |
| 6 | 3 | 16 | T | T/A | G | C | C | G | C | A | C | G/A | T |
| 7 | 11 | 1 | T | T | G | C | C | G | C | A | C | G | T |
| 8 | 11 | 2 | T | T | G | C | C | G | C | A | C | G | T |
| 9 | 11 | 3 | T | T | G | C | C | G | C | A | C | G | T |
| 10 | 11 | 4 | T | T | G | C | C/G | G | C | A/G | C | G | T |
| 11 | 11 | 5 | T | T | G | C | C | G | C | A | C | G | T |
| 12 | 11 | 6 | T | T | G | C | C/G | G | C | A/G | C | G | T |
| 13 | 11 | 7 | T | T/A | G | C | C | G | C | A | C | G | T |
| 14 | 11 | 8 | T | T | G | C | C | G | C | A | C | G | T |
| 15 | 11 | 9 | T/G | T | G/A | C/T | C | G/A | C | A | C/T | G | T |
| 16 | 11 | 10 | T | T | G | C | C | G | C | A | C | G | T/A |
| 17 | 11 | 11 | T | T | G | C | C | G | C | A | C | G | T |
| 18 | 11 | 12 | T | T | G | C | C | G | C/T | A | C | G | T |
| 19 | 11 | 15 | T | T/A | G | C | C | C | G | A | C | G | T |
| 20 | 11 | 16 | T | T/A | G | C | C | G | C | A | C | G/A | T |

The haplotype pairs shown in Table 3 were estimated from the unphased genotypes using a computer-implemented algorithm for assigning haplotypes to unrelated individuals in a population sample, as described in WO 01/80156. In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals. In the present analysis, the list of haplotypes was augmented with haplotypes obtained from two families (one three-generation Caucasian family and one two-generation African-American family).

By following this protocol, it was determined that the Index Repository examined herein and, by extension, the general population contains the 16 human UCP2 haplotypes shown in Table 4 below, wherein each of the UCP2 haplotypes comprises a 5′-3′ ordered sequence of 23 polymorphisms whose positions in SEQ ID NO:1 and alleles are set forth in Table 4. In Table 4, the column labeled “Region Examined” provides the nucleotide positions in SEQ ID NO:1 corresponding to sequenced regions of the gene. The columns labeled “PS No.” and “PS Position” provide the polymorphic site number designation (see Table 2) and the corresponding nucleotide position of this polymorphic site within SEQ ID NO:1 or SEQ ID NO:116. The columns beneath the “Haplotype Number” heading are labeled to provide a unique number designation for each UCP2 haplotype.

TABLE 4
Haplotypes of the UCP2 gene.

| Region | PS | PS | Haplotype Number(d) | | | | | | | |
|---|---|---|---|---|---|---|---|---|---|---|
| Examined(a) | No.(b) | Position(c) | 1 | 2 | 3 | 4 | 5 | 6 | 7 | 8 |
| 1000-2531 | 1 | 1283/30 | C | C | C | C | C | C | C | G |
| 1000-2531 | 2 | 1714/150 | C | C | C | C | C | C | C | C |
| 1000-2531 | 3 | 2051/270 | T | T | T | T | T | T | T | C |
| 1000-2531 | 4 | 2124/390 | C | C | C | C | C | C | C | C |
| 1000-2531 | 5 | 2287/510 | C | C | C | C | C | C | G | C |
| 1000-2531 | 6 | 2408/630 | A | A | A | A | A | G | A | A |
| 4393-5236 | 7 | 4768/750 | A | A | A | A | G | A | A | A |
| 4393-5236 | 8 | 4785/870 | A | G | G | G | G | G | G | G |
| 4393-5236 | 9 | 4813/990 | T | T | T | T | C | T | T | T |
| 4393-5236 | 10 | 4882/1110 | A | A | A | A | C | A | A | A |
| 4393-5236 | 11 | 4976/1230 | T | T | T | T | T | T | T | A |
| 5399-5908 | 12 | 5600/1350 | T | C | T | T | T | T | C | C |
| 5399-5908 | 13 | 5820/1470 | T | T | T | T | T | T | T | T |
| 6413-7225 | 14 | 6536/1590 | T | T | T | T | T | T | A | T |
| 6413-7225 | 15 | 6607/1710 | G | G | G | G | G | G | G | G |
| 6413-7225 | 16 | 6617/1830 | C | C | C | C | C | C | C | C |
| 6413-7225 | 17 | 6872/1950 | C | C | C | G | C | G | C | C |
| 6413-7225 | 18 | 6966/2070 | G | G | G | G | G | G | G | G |
| 6413-7225 | 19 | 7036/2190 | C | C | C | C | C | C | C | C |
| 6413-7225 | 20 | 7086/2310 | A | A | A | G | A | G | A | A |
| 7764-8311 | 21 | 8100/2430 | C | C | C | C | C | C | C | C |
| 7764-8311 | 22 | 8221/2550 | G | C | C | C | C | C | G | G |
| 8367-8792 | 23 | 8677/2670 | T | T | T | T | T | T | T | T |
| Region | PS | PS | Haplotype Number(d) | | | | | | | |
| Examined(a) | No.(b) | Position(c) | 9 | 10 | 11 | 12 | 13 | 14 | 15 | 16 |
| 1000-2531 | 1 | 1283/30 | G | G | G | G | G | G | G | G |
| 1000-2531 | 2 | 1714/150 | C | C | C | C | C | C | T | T |
| 1000-2531 | 3 | 2051/270 | T | T | T | T | T | T | T | T |
| 1000-2531 | 4 | 2124/390 | C | C | C | C | C | C | C | T |
| 1000-2531 | 5 | 2287/510 | C | C | C | C | C | C | G | C |
| 1000-2531 | 6 | 2408/630 | A | A | A | A | A | A | A | A |
| 4393-5236 | 7 | 4768/750 | A | A | A | A | A | A | A | A |
| 4393-5236 | 8 | 4785/870 | G | G | G | G | G | G | G | G |
| 4393-5236 | 9 | 4813/990 | T | T | T | T | T | T | T | T |
| 4393-5236 | 10 | 4882/1110 | A | A | A | A | A | A | A | A |
| 4393-5236 | 11 | 4976/1230 | T | T | T | T | T | T | T | T |
| 5399-5908 | 12 | 5600/1350 | C | C | C | C | C | T | C | C |
| 5399-5908 | 13 | 5820/1470 | G | T | T | T | T | T | T | T |
| 6413-7225 | 14 | 6536/1590 | T | T | T | T | T | T | A | A |
| 6413-7225 | 15 | 6607/1710 | A | G | G | G | G | G | G | G |
| 6413-7225 | 16 | 6617/1830 | T | C | C | C | T | C | C | C |
| 6413-7225 | 17 | 6872/1950 | C | C | C | C | C | C | C | C |
| 6413-7225 | 18 | 6966/2070 | A | G | G | G | A | G | G | G |
| 6413-7225 | 19 | 7036/2190 | C | C | C | T | C | C | C | C |
| 6413-7225 | 20 | 7086/2310 | A | A | A | A | A | A | A | A |
| 7764-8311 | 21 | 8100/2430 | T | C | C | C | C | C | C | C |
| 7764-8311 | 22 | 8221/2550 | G | G | G | G | G | G | G | A |
| 8367-8792 | 23 | 8677/2670 | T | A | T | T | T | T | T | T |

(a)Region examined represents the nucleotide positions defining the start and stop positions within SEQ ID NO: 1 of the regions sequenced;
(b)PS = polymorphic site;
(c)Position of PS within the indicated SEQ ID NO, with the 1^{st} position number referring to SEQ ID NO: 1 and the 2^{nd} position number referring to SEQ ID NO: 116, a modified version of SEQ ID NO: 1 that comprises the context sequence of each polymorphic site, PS1-PS23, to facilitate electronic searching of the haplotypes;
(d)Alleles for UCP2 haplotypes are presented 5′ to 3′ in each column.

SEQ ID NO:1 refers to FIG. 1, with the two alternative allelic variants of each polymorphic site indicated by the appropriate nucleotide symbol. SEQ ID NO:116 is a modified version of SEQ ID NO:1 that shows the context sequence of each of PS1-PS23 in a uniform format to facilitate electronic searching of the UCP2 haplotypes. For each polymorphic site, SEQ ID NO:116 contains a block of 60 bases of the nucleotide sequence encompassing the centrally-located polymorphic site at the 30^{th} position, followed by 60 bases of unspecified sequence to represent that each polymorphic site is separated by genomic sequence whose composition is defined elsewhere herein.

Table 5 below shows the number of chromosomes characterized by a given UCP2 haplotype for all unrelated individuals in the Index Repository for which haplotype data was obtained. The number of these unrelated individuals who have a given UCP2 haplotype pair is shown in Table 6. In Tables 5 and 6, the “Total” column shows this frequency data for all of these unrelated individuals, while the other columns show the frequency data for these unrelated individuals categorized according to their self-identified ethnogeographic origin. Abbreviations used in Tables 5 and 6 are AF=African Descent, AS=Asian, CA=Caucasian, HL=Hispanic-Latino, and AM=Native American.

TABLE 5
Frequency of Observed UCP2 Haplotypes In Unrelated Individuals

| HAP No. | HAP ID | Total | CA | AF | AS | HL | AM |
|---|---|---|---|---|---|---|---|
| 1 | 510345956 | 1 | 0 | 0 | 0 | 1 | 0 |
| 2 | 510345917 | 1 | 0 | 0 | 0 | 1 | 0 |
| 3 | 510345795 | 56 | 17 | 11 | 12 | 13 | 3 |
| 4 | 510345833 | 3 | 0 | 3 | 0 | 0 | 0 |
| 5 | 510345902 | 1 | 1 | 0 | 0 | 0 | 0 |
| 6 | 510346017 | 1 | 0 | 1 | 0 | 0 | 0 |
| 7 | 510345978 | 1 | 0 | 1 | 0 | 0 | 0 |
| 8 | 510345969 | 1 | 0 | 1 | 0 | 0 | 0 |
| 9 | 510345986 | 1 | 0 | 1 | 0 | 0 | 0 |
| 10 | 510345938 | 1 | 0 | 1 | 0 | 0 | 0 |
| 11 | 510345747 | 82 | 20 | 16 | 27 | 16 | 3 |
| 12 | 510345866 | 2 | 0 | 1 | 1 | 0 | 0 |
| 13 | 510345850 | 2 | 0 | 2 | 0 | 0 | 0 |
| 14 | 510345882 | 1 | 0 | 1 | 0 | 0 | 0 |
| 15 | 510346006 | 1 | 0 | 1 | 0 | 0 | 0 |
| 16 | 510345811 | 9 | 4 | 0 | 0 | 5 | 0 |

TABLE 6
Frequency of Observed UCP2 Haplotype Pairs In Unrelated Individuals

| HAP1 | HAP2 | Total | CA | AF | AS | HL | AM |
|---|---|---|---|---|---|---|---|
| 3 | 3 | 11 | 4 | 2 | 3 | 1 | 1 |
| 3 | 4 | 1 | 0 | 1 | 0 | 0 | 0 |
| 3 | 12 | 1 | 0 | 1 | 0 | 0 | 0 |
| 3 | 13 | 2 | 0 | 2 | 0 | 0 | 0 |
| 3 | 14 | 1 | 0 | 1 | 0 | 0 | 0 |
| 3 | 16 | 2 | 0 | 0 | 0 | 2 | 0 |
| 11 | 1 | 1 | 0 | 0 | 0 | 1 | 0 |
| 11 | 2 | 1 | 0 | 0 | 0 | 1 | 0 |
| 11 | 3 | 27 | 9 | 2 | 6 | 9 | 1 |
| 11 | 4 | 2 | 0 | 2 | 0 | 0 | 0 |
| 11 | 5 | 1 | 1 | 0 | 0 | 0 | 0 |
| 11 | 6 | 1 | 0 | 1 | 0 | 0 | 0 |
| 11 | 7 | 1 | 0 | 1 | 0 | 0 | 0 |
| 11 | 8 | 1 | 0 | 1 | 0 | 0 | 0 |
| 11 | 9 | 1 | 0 | 1 | 0 | 0 | 0 |
| 11 | 10 | 1 | 0 | 1 | 0 | 0 | 0 |
| 11 | 11 | 18 | 3 | 3 | 10 | 1 | 1 |
| 11 | 12 | 1 | 0 | 0 | 1 | 0 | 0 |
| 11 | 15 | 1 | 0 | 1 | 0 | 0 | 0 |
| 11 | 16 | 7 | 4 | 0 | 0 | 3 | 0 |

The size and composition of the Index Repository were chosen to represent the genetic diversity across and within four major population groups comprising the general United States population. For example, as described in Table 1 above, this repository contains approximately equal sample sizes of African-descent, Asian-American, European-American, and Hispanic-Latino population groups. Almost all individuals representing each group had all four grandparents with the same ethnogeographic background. The number of unrelated individuals in the Index Repository provides a sample size that is sufficient to detect SNPs and haplotypes that occur in the general population with high statistical certainty. For instance, a haplotype that occurs with a frequency of 5% in the general population has a probability higher than 99.9% of being observed in a sample of 80 individuals from the general population. Similarly, a haplotype that occurs with a frequency of 10% in a specific population group has a 99% probability of being observed in a sample of 20 individuals from that population group. In addition, the size and composition of the Index Repository means that the relative frequencies determined therein for the haplotypes and haplotype pairs of the UCP2 gene are likely to be similar to the relative frequencies of these UCP2 haplotypes and haplotype pairs in the general U.S. population and in the four population groups represented in the Index Repository. The genetic diversity observed for the three Native Americans is presented because it is of scientific interest, but due to the small sample size it lacks statistical significance.

Each UCP2 haplotype shown in Table 4 defines an UCP2 isogene. The UCP2 isogene defined by a given UCP2 haplotype comprises the examined regions of SEQ ID NO:1 indicated in Table 4, with the corresponding ordered sequence of nucleotides occurring at each polymorphic site within the UCP2 gene shown in Table 4 for that defining haplotype.

Each UCP2 isogene defined by one of the haplotypes shown in Table 4 will further correspond to a particular UCP2 coding sequence variant. Each of these UCP2 coding sequence variants comprises the regions of SEQ ID NO:2 examined and is defined by the 5′-3′ ordered sequence of nucleotides occurring at each polymorphic site within the coding sequence of the UCP2 gene, as shown in Table 7. In Table 7, the column labeled ‘Region Examined’ provides the nucleotide positions in SEQ ID NO:2 corresponding to sequenced regions of the gene; the columns labeled ‘PS No.’ and ‘PS Position’ provide the polymorphic site number designation (see Table 2) and the corresponding nucleotide position of this polymorphic site within SEQ ID NO:2. The columns beneath the ‘Coding Sequence Number’ heading are numbered to correspond to the haplotype number defining the UCP2 isogene from which the coding sequence variant is derived. UCP2 coding sequence variants that differ from the reference UCP2 coding sequence are denoted in Table 7 by a letter (A, B, etc) identifying each unique novel coding sequence. The same letter at the top of more than one column denotes that a given novel coding sequence is present in multiple novel UCP2 isogenes.

TABLE 7
Nucleotides Present at Polymorphic Sites Within the Observed UCP2
Coding Sequences

| Region | PS | PS | Coding Sequence Number(d) | | | | | | | |
|---|---|---|---|---|---|---|---|---|---|---|
| Examined(a) | No.(b) | Position(c) | 1A | 2 | 3A | 4A | 5A | 6A | 7 | 8 |
| 119-930 | 12 | 164 | T | C | T | T | T | T | C | C |
| 119-930 | 18 | 582 | G | G | G | G | G | G | G | G |
| 119-930 | 21 | 750 | C | C | C | C | C | C | C | C |

| Region | | PS | | | | | | | | |
|---|---|---|---|---|---|---|---|---|---|---|
| Ex- | PS | Posi- | Coding Sequence Number(d) | | | | | | | |
| amined(a) | No.(b) | tion(c) | 9B | 10 | 11 | 12 | 13C | 14A | 15 | 16 |
| 119-930 | 12 | 164 | C | C | C | C | C | T | C | C |
| 119-930 | 18 | 582 | A | G | G | G | A | G | G | G |
| 119-930 | 21 | 750 | T | C | C | C | C | C | C | C |

(a)Region examined represents the nucleotide positions in SEQ ID NO: 2 defining the start and stop positions of the regions sequenced;
(b)PS = polymorphic site;
(c)Position of PS within SEQ ID NO: 2;
(d)Alleles for UCP2 coding sequences are presented 5′ to 3′ in each column. The number at the top of each column designates the haplotype number of the UCP2 isogene from which the coding sequence is derived. UCP2 coding sequences that differ from the reference are denoted in this table by a letter following the isogene number.

In view of the above, it will be seen that the several advantages of the invention are achieved and other advantageous results attained.

For any and all embodiments of the present invention discussed herein, in which a feature is described in terms of a Markush group or other grouping of alternatives, the inventors contemplate that such feature may also be described by, and that their invention specifically includes, any individual member or subgroup of members of such Markush group or other group.

As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinency of the cited references.

What is claimed is:

1. A method for haplotyping the uncoupling protein 2 (mitochondrial, proton carrier) (UCP2) gene of an individual, which comprises identifying the phased sequence of nucleotides at PS1-PS23 for at least one copy of the individual's UCP2 gene and assigning to the individual an UCP2 haplotype that is consistent with the phased sequence, wherein the assigned UCP2 haplotype comprises a haplotype selected from the group consisting of the UCP2 haplotypes shown in the table immediately below:

| PS | PS | Haplotype Number(c) (Part 1) | | | | | | | |
|---|---|---|---|---|---|---|---|---|---|
| No.(a) | Position(b) | 1 | 2 | 3 | 4 | 5 | 6 | 7 | 8 |
| 1 | 1283 | C | C | C | C | C | C | C | G |
| 2 | 1714 | C | C | C | C | C | C | C | C |
| 3 | 2051 | T | T | T | T | T | T | T | C |
| 4 | 2124 | C | C | C | C | C | C | C | C |
| 5 | 2287 | C | C | C | C | C | C | G | C |
| 6 | 2408 | A | A | A | A | A | G | A | A |
| 7 | 4768 | A | A | A | A | G | A | A | A |
| 8 | 4785 | A | G | G | G | G | G | G | C |
| 9 | 4813 | T | T | T | T | C | T | T | T |
| 10 | 4882 | A | A | A | A | C | A | A | A |
| 11 | 4976 | T | T | T | T | T | T | T | A |
| 12 | 5600 | T | C | T | T | T | T | C | C |
| 13 | 5820 | T | T | T | T | T | T | T | T |
| 14 | 6536 | T | T | T | T | T | T | A | T |
| 15 | 6607 | G | G | G | G | G | G | G | G |
| 16 | 6617 | C | C | C | C | C | C | C | C |
| 17 | 6872 | C | C | C | G | C | G | C | C |
| 18 | 6966 | G | G | G | G | G | G | G | G |
| 19 | 7036 | C | C | C | C | C | C | C | C |
| 20 | 7086 | A | A | A | G | A | G | A | A |
| 21 | 8100 | C | C | C | C | C | C | C | C |
| 22 | 8221 | G | G | G | G | G | G | G | G |
| 23 | 8677 | T | T | T | T | T | T | T | T |
| PS | PS | Haplotype Number(c) (Part 2) | | | | | | | |
| No.(a) | Position(b) | 9 | 10 | 11 | 12 | 13 | 14 | 15 | 16 |
| 1 | 1283 | G | G | G | G | G | G | G | G |
| 2 | 1714 | C | C | C | C | C | C | T | T |
| 3 | 2051 | T | T | T | T | T | T | T | T |
| 4 | 2124 | C | C | C | C | C | C | C | T |
| 5 | 2287 | C | C | C | C | C | C | G | C |
| 6 | 2408 | A | A | A | A | A | A | A | A |
| 7 | 4768 | A | A | A | A | A | A | A | A |
| 8 | 4785 | G | G | G | G | G | G | G | G |
| 9 | 4813 | T | T | T | T | T | T | T | T |
| 10 | 4882 | A | A | A | A | A | A | A | A |
| 11 | 4976 | T | T | T | T | T | T | T | T |
| 12 | 5600 | C | C | C | C | C | T | C | C |
| 13 | 5820 | G | T | T | T | T | T | T | T |
| 14 | 6536 | T | T | T | T | T | T | A | A |
| 15 | 6607 | A | G | G | G | G | G | G | G |
| 16 | 6617 | T | C | C | C | T | C | C | C |
| 17 | 6872 | C | C | C | C | C | C | C | C |
| 18 | 6966 | A | G | G | G | A | G | G | G |
| 19 | 7036 | C | C | C | T | C | C | C | C |
| 20 | 7086 | A | A | A | A | A | A | A | A |
| 21 | 8100 | T | C | C | C | C | C | C | C |
| 22 | 8221 | G | G | G | G | G | G | G | A |
| 23 | 8677 | T | A | T | T | T | T | T | T |

(a)PS = polymorphic site;
(b)Position of PS within SEQ ID NO: 1;
(c)Alleles for haplotypes are presented 5′ to 3′ in each column.

2. A method for haplotyping the uncoupling protein 2 (mitochondrial, proton carrier) (UCP2) gene of an individual, which comprises identifying the phased sequence of nucleotides at PS1-PS23 for each copy of the individual's UCP2 gene and assigning to the individual an UCP2 haplotype pair that is consistent with each of the phased sequences, wherein the assigned UCP2 haplotype pair comprises a haplotype pair selected from the group consisting of the UCP2 haplotype pairs shown in the table immediately below:

| PS | PS | Haplotype Pair(c) (Part 1) | | | | | | | | | |
|---|---|---|---|---|---|---|---|---|---|---|---|
| No.(a) | Position(b) | 3/3 | 3/4 | 3/12 | 3/13 | 3/14 | 3/16 | 11/1 | 11/2 | 11/3 | 11/4 |
| 1 | 1283 | C/C | C/C | C/G | C/G | C/G | C/G | G/C | G/C | G/G | G/C |
| 2 | 1714 | C/C | C/C | C/C | C/C | C/C | C/T | C/C | C/C | C/C | C/C |
| 3 | 2051 | T/T | T/T | T/T | T/T | T/T | T/T | T/T | T/T | T/T | T/T |
| 4 | 2124 | C/C | C/C | C/C | C/C | C/C | C/T | C/C | C/C | C/C | C/C |
| 5 | 2287 | C/C | C/C | C/C | C/C | C/C | C/C | C/C | C/C | C/C | C/C |
| 6 | 2408 | A/A | A/A | A/A | A/A | A/A | A/A | A/A | A/A | A/A | A/A |
| 7 | 4768 | A/A | A/A | A/A | A/A | A/A | A/A | A/A | A/A | A/A | A/A |
| 8 | 4785 | G/G | G/G | G/G | G/G | G/G | G/G | G/A | G/G | G/G | G/G |
| 9 | 4813 | T/T | T/T | T/T | T/T | T/T | T/T | T/T | T/T | T/T | T/T |
| 10 | 4882 | A/A | A/A | A/A | A/A | A/A | A/A | A/A | A/A | A/A | A/A |
| 11 | 4976 | T/T | T/T | T/T | T/T | T/T | T/T | T/T | T/T | T/T | T/T |
| 12 | 5600 | T/T | T/T | T/C | T/C | T/T | T/C | C/T | C/C | C/T | C/T |
| 13 | 5820 | T/T | T/T | T/T | T/T | T/T | T/T | T/T | T/T | T/T | T/T |
| 14 | 6536 | T/T | T/T | T/T | T/T | T/T | T/A | T/T | T/T | T/T | T/T |
| 15 | 6607 | G/G | G/G | G/G | G/G | G/G | G/G | G/G | G/G | G/G | G/G |
| 16 | 6617 | C/C | C/C | C/C | C/T | C/C | C/C | C/C | C/C | C/C | C/C |
| 17 | 6872 | C/C | C/G | C/C | C/C | C/C | C/C | C/C | C/C | C/C | C/G |
| 18 | 6966 | G/G | G/G | G/G | G/A | G/G | G/G | G/G | G/G | G/G | G/G |
| 19 | 7036 | C/C | C/C | C/T | C/C | C/C | C/C | C/C | C/C | C/C | C/C |
| 20 | 7086 | A/A | A/G | A/A | A/A | A/A | A/A | A/A | A/A | A/A | A/G |
| 21 | 8100 | C/C | C/C | C/C | C/C | C/C | C/C | C/C | C/C | C/C | C/C |
| 22 | 8221 | G/G | G/G | G/G | G/G | G/G | G/A | G/G | G/G | G/G | G/G |
| 23 | 8677 | T/T | T/T | T/T | T/T | T/T | T/T | T/T | T/T | T/T | T/T |
| PS | PS | Haplotype Pair(c) (Part 2) | | | | | | | | | |
| No.(a) | Position(b) | 11/5 | 11/6 | 11/7 | 11/8 | 11/9 | 11/10 | 11/11 | 11/12 | 11/15 | 11/16 |
| 1 | 1283 | G/C | G/C | G/C | G/G | G/G | G/G | G/G | G/G | G/G | G/G |
| 2 | 1714 | C/C | C/C | C/C | C/C | C/C | C/C | C/C | C/C | C/T | C/T |
| 3 | 2051 | T/T | T/T | T/T | T/C | T/T | T/T | T/T | T/T | T/T | T/T |
| 4 | 2124 | C/C | C/C | C/C | C/C | C/C | C/C | C/C | C/C | C/C | C/T |
| 5 | 2287 | C/C | C/C | C/G | C/C | C/C | C/C | C/C | C/C | C/G | C/C |
| 6 | 2408 | A/A | A/G | A/A | A/A | A/A | A/A | A/A | A/A | A/A | A/A |
| 7 | 4768 | A/G | A/A | A/A | A/A | A/A | A/A | A/A | A/A | A/A | A/A |
| 8 | 4785 | G/G | G/G | G/G | G/G | G/G | G/G | G/G | G/G | G/G | G/G |
| 9 | 4813 | T/C | T/T | T/T | T/T | T/T | T/T | T/T | T/T | T/T | T/T |
| 10 | 4882 | A/C | A/A | A/A | A/A | A/A | A/A | A/A | A/A | A/A | A/A |
| 11 | 4976 | T/T | T/T | T/T | T/A | T/T | T/T | T/T | T/T | T/T | T/T |
| 12 | 5600 | C/T | C/T | C/C | C/C | C/C | C/C | C/C | C/C | C/C | C/C |
| 13 | 5820 | T/T | T/T | T/T | T/T | T/G | T/T | T/T | T/T | T/T | T/T |
| 14 | 6536 | T/T | T/T | T/A | T/T | T/T | T/T | T/T | T/T | T/A | T/A |
| 15 | 6607 | G/G | G/G | G/G | G/G | G/A | G/G | G/G | G/G | G/G | G/G |
| 16 | 6617 | C/C | C/C | C/C | C/C | C/T | C/C | C/C | C/C | C/C | C/C |
| 17 | 6872 | C/C | G/G | C/C | C/C | C/C | C/C | C/C | C/C | C/C | C/C |
| 18 | 6966 | G/G | G/G | G/G | G/G | G/G | G/G | G/G | G/G | G/G | G/G |
| 19 | 7036 | C/C | C/C | C/C | C/C | C/C | C/C | C/C | C/T | C/C | C/C |
| 20 | 7086 | A/A | A/G | A/A | A/A | A/A | A/A | A/A | A/A | A/A | A/A |
| 21 | 8100 | C/C | C/C | C/C | C/C | C/T | C/C | C/C | C/C | C/C | C/C |
| 22 | 8221 | G/G | G/G | G/G | G/G | G/G | G/G | G/G | G/G | G/G | G/A |
| 23 | 8677 | T/T | T/T | T/T | T/T | T/T | T/A | T/T | T/T | T/T | T/T |

(a)PS = polymorphic site;
(b)Position of PS in SEQ ID NO: 1;
(c)Haplotype pairs are represented as 1^{st} haplotype/2^{nd} haplotype; with alleles of each haplotype shown 5′ to 3′ as 1^{st} polymorphism/2^{nd} polymorphism in each column.

3. A method for genotyping the uncoupling protein 2 (mitochondrial, proton carrier) (UCP2) gene of an individual, comprising determining for the two copies of the UCP2 gene present in the individual the identity of the nucleotide pair at one or more polymorphic sites (PS) selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23, wherein the one or more polymorphic sites (PS) have the position and alternative alleles shown in SEQ ID NO:1.

4. The method of claim 3, which comprises determining for the two copies of the UCP2 gene present in the individual the identity of the nucleotide pair at each of PS1-PS23.

5. A method for haplotyping the uncoupling protein 2 (mitochondrial, proton carrier) (UCP2) gene of an individual which comprises determining, for one copy of the UCP2 gene present in the individual, the identity of the nucleotide at two or more polymorphic sites (PS) selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23, wherein the selected PS have the position and alternative alleles shown in SEQ ID NO:1.

6. The method of claim 6, further comprising determining the identity of the nucleotide at PS12, wherein the PS has the position and alternative alleles shown in SEQ ID NO:1.

7. A method for assigning a haplotype pair for the uncoupling protein 2 (mitochondrial, proton carrier) (UCP2) gene to an individual comprising:
(a) identifying an UCP2 genotype for the individual, wherein the genotype comprises the nucleotide pair at two or more polymorphic sites (PS) selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23, wherein the selected PS have the position and alternative alleles shown in SEQ ID NO:1;
(b) comparing the genotype to haplotype pair data for the UCP2 gene, wherein the haplotype pair data comprise the haplotype pair data set forth in the table immediately below; and
(c) assigning to the individual a haplotype pair that is consistent with the genotype of the individual and with the haplotype pair data

| PS | PS | Haplotype Pair(c) (Part 1) | | | | | | | | | |
|---|---|---|---|---|---|---|---|---|---|---|---|
| No.(a) | Position(b) | 3/3 | 3/4 | 3/12 | 3/13 | 3/14 | 3/16 | 11/1 | 11/2 | 11/3 | 11/4 |
| 1 | 1283 | C/C | C/C | C/G | C/G | C/G | C/G | G/C | G/C | G/C | G/C |
| 2 | 1714 | C/C | C/C | C/C | C/C | C/C | C/T | C/C | C/C | C/C | C/C |
| 3 | 2051 | T/T | T/T | T/T | T/T | T/T | T/T | T/T | T/T | T/T | T/T |
| 4 | 2124 | C/C | C/C | C/C | C/C | C/C | C/T | C/C | C/C | C/C | C/C |
| 5 | 2287 | C/C | C/C | C/C | C/C | C/C | C/C | C/C | C/C | C/C | C/C |
| 6 | 2408 | A/A | A/A | A/A | A/A | A/A | A/A | A/A | A/A | A/A | A/A |
| 7 | 4768 | A/A | A/A | A/A | A/A | A/A | A/A | A/A | A/A | A/A | A/A |
| 8 | 4785 | G/G | G/G | G/G | G/G | G/G | G/G | G/A | G/G | G/G | G/G |
| 9 | 4813 | T/T | T/T | T/T | T/T | T/T | T/T | T/T | T/T | T/T | T/T |
| 10 | 4882 | A/A | A/A | A/A | A/A | A/A | A/A | A/A | A/A | A/A | A/A |
| 11 | 4976 | T/T | T/T | T/T | T/T | T/T | T/T | T/T | T/T | T/T | T/T |
| 12 | 5600 | T/T | T/T | T/C | T/C | T/T | T/C | C/T | C/C | C/T | C/T |
| 13 | 5820 | T/T | T/T | T/T | T/T | T/T | T/T | T/T | T/T | T/T | T/T |
| 14 | 6536 | T/T | T/T | T/T | T/T | T/T | T/A | T/T | T/T | T/T | T/T |
| 15 | 6607 | G/G | G/G | G/G | G/G | G/G | G/G | G/G | G/G | G/G | G/G |
| 16 | 6617 | C/C | C/C | C/C | C/T | C/C | C/C | C/C | C/C | C/C | C/C |
| 17 | 6872 | C/C | C/G | C/C | C/C | C/C | C/C | C/C | C/C | C/C | C/G |
| 18 | 6966 | G/G | G/G | G/G | G/G | G/G | G/G | G/G | G/G | G/G | G/G |
| 19 | 7036 | C/C | C/C | C/T | C/C | C/C | C/C | C/C | C/C | C/C | C/C |
| 20 | 7086 | A/A | A/G | A/A | A/A | A/A | A/A | A/A | A/A | A/A | A/G |
| 21 | 8100 | C/C | C/C | C/C | C/C | C/C | C/C | C/C | C/C | C/C | C/C |
| 22 | 8221 | G/G | G/G | G/G | G/G | G/G | G/A | G/G | G/G | G/G | G/G |
| 23 | 8677 | T/T | T/T | T/T | T/T | T/T | T/T | T/T | T/T | T/T | T/T |
| PS | PS | Haplotype Pair(c) (Part 2) | | | | | | | | | |
| No.(a) | Position(b) | 11/5 | 11/6 | 11/7 | 11/8 | 11/9 | 11/10 | 11/11 | 11/12 | 11/15 | 11/16 |
| 1 | 1283 | G/C | G/C | G/C | G/G | G/G | G/G | G/G | G/G | G/G | G/C |
| 2 | 1714 | C/C | C/C | C/C | C/C | C/C | C/C | C/C | C/C | C/T | C/T |
| 3 | 2051 | T/T | T/T | T/T | T/C | T/T | T/C | T/T | T/T | T/T | T/T |
| 4 | 2124 | C/C | C/C | C/C | C/C | C/C | C/C | C/C | C/C | C/C | C/T |
| 5 | 2287 | C/C | C/C | C/G | C/C | C/C | C/C | C/C | C/C | C/G | C/C |
| 6 | 2408 | A/A | A/G | A/A | A/G | A/A | A/A | A/A | A/A | A/A | A/A |
| 7 | 4768 | A/G | A/A | A/A | A/A | A/A | A/A | A/A | A/A | A/A | A/A |
| 8 | 4785 | G/G | G/G | G/G | G/G | G/G | G/G | G/G | G/G | G/G | G/G |
| 9 | 4813 | T/C | T/T | T/C | T/T | T/T | T/T | T/T | T/T | T/T | T/T |
| 10 | 4882 | A/C | A/A | A/A | A/A | A/A | A/A | A/A | A/A | A/A | A/A |
| 11 | 4976 | T/T | T/T | T/T | T/A | T/T | T/T | T/T | T/T | T/T | T/T |
| 12 | 5600 | C/T | C/T | C/C | C/C | C/C | C/C | C/C | C/C | C/C | C/C |
| 13 | 5820 | T/T | T/T | T/T | T/T | T/T | T/T | T/T | T/T | T/T | T/T |
| 14 | 6536 | T/T | T/T | T/A | T/T | T/T | T/T | T/T | T/T | T/A | T/A |
| 15 | 6607 | G/G | G/G | G/G | G/G | G/A | G/G | G/G | G/G | G/G | G/G |
| 16 | 6617 | C/C | C/C | C/C | C/T | C/T | C/C | C/C | C/C | C/C | C/C |
| 17 | 6872 | C/C | C/G | C/C | C/C | C/C | C/C | C/C | C/C | C/C | C/C |
| 18 | 6966 | G/G | G/G | G/G | G/G | G/A | G/G | G/G | G/G | G/G | G/G |
| 19 | 7036 | C/C | C/C | C/C | C/C | C/C | C/C | C/C | C/T | C/C | C/C |
| 20 | 7086 | A/A | A/G | A/A | A/A | A/A | A/A | A/A | A/A | A/A | A/A |
| 21 | 8100 | C/C | C/C | C/C | C/C | C/T | C/C | C/C | C/C | C/C | C/C |
| 22 | 8221 | G/G | G/G | G/G | G/G | G/G | G/G | G/G | G/G | G/G | G/A |
| 23 | 8677 | T/T | T/T | T/T | T/T | T/T | T/T | T/T | T/T | T/T | T/T |

(a)PS = polymorphic site;
(b)Position of PS in SEQ ID NO: 1;
(c)Haplotype pairs are represented as 1^{st} haplotype/2^{nd} haplotype; with alleles of each haplotype shown 5′ to 3′ as 1^{st} polymorphism/2^{nd} polymorphism in each column.

8. The method of claim 7, wherein the identified genotype of the individual comprises the nucleotide pair at each of PS1-PS23, which have the position and alternative alleles shown in SEQ ID NO:1.

9. A method for identifying an association between a trait and at least one haplotype or haplotype pair of the uncoupling protein 2 (mitochondrial, proton carrier) (UCP2) gene which comprises comparing the frequency of the haplotype or haplotype pair in a population exhibiting the trait with the frequency of the haplotype or haplotype pair in a reference population, wherein the haplotype is selected from haplotypes 1-16 shown in the table presented immediately below:

| PS | PS | Haplotype Number(c) (Part 1) | | | | | | | |
|---|---|---|---|---|---|---|---|---|---|
| No.(a) | Position(b) | 1 | 2 | 3 | 4 | 5 | 6 | 7 | 8 |
| 1 | 1283 | C | C | C | C | C | C | C | G |
| 2 | 1714 | C | C | C | C | C | C | C | C |
| 3 | 2051 | T | T | T | T | T | T | T | C |
| 4 | 2124 | C | C | C | C | C | C | C | C |
| 5 | 2287 | C | C | C | C | C | C | G | C |
| 6 | 2408 | A | A | A | A | A | G | A | A |
| 7 | 4768 | A | A | A | A | G | A | A | A |
| 8 | 4785 | A | G | G | G | G | G | G | G |
| 9 | 4813 | T | T | T | T | C | T | T | T |
| 10 | 4882 | A | A | A | A | C | A | A | A |
| 11 | 4976 | T | T | T | T | T | T | T | A |
| 12 | 5600 | T | C | T | T | T | T | C | C |
| 13 | 5820 | T | T | T | T | T | T | T | T |
| 14 | 6536 | T | T | T | T | T | T | A | T |
| 15 | 6607 | G | G | G | G | G | G | G | G |
| 16 | 6617 | C | C | C | C | C | C | C | C |
| 17 | 6872 | C | C | C | G | C | G | C | C |
| 18 | 6966 | G | G | G | G | G | G | G | G |
| 19 | 7036 | C | C | C | C | C | C | C | C |
| 20 | 7086 | A | A | A | G | A | G | A | A |
| 21 | 8100 | C | C | C | C | C | C | C | C |
| 22 | 8221 | G | G | G | G | G | G | G | G |
| 23 | 8677 | T | T | T | T | T | T | T | T |
| PS | PS | Haplotype Number(c) (Part 2) | | | | | | | |
| No.(a) | Position(b) | 9 | 10 | 11 | 12 | 13 | 14 | 15 | 16 |
| 1 | 1283 | G | G | G | G | G | G | G | G |
| 2 | 1714 | C | C | C | C | C | C | T | T |
| 3 | 2051 | T | T | T | T | T | T | T | T |
| 4 | 2124 | C | C | C | C | C | C | C | T |
| 5 | 2287 | C | C | C | C | C | C | G | C |
| 6 | 2408 | A | A | A | A | A | A | A | A |
| 7 | 4768 | A | A | A | A | A | A | A | A |
| 8 | 4785 | G | G | G | G | G | G | G | G |
| 9 | 4813 | T | T | T | T | T | T | T | T |
| 10 | 4882 | A | A | A | A | A | A | A | A |
| 11 | 4976 | T | T | T | T | T | T | T | T |
| 12 | 5600 | C | C | C | C | C | T | C | C |
| 13 | 5820 | G | T | T | T | T | T | T | T |
| 14 | 6536 | T | T | T | T | T | T | A | A |
| 15 | 6607 | A | G | G | G | G | G | G | G |
| 16 | 6617 | T | C | C | C | T | C | C | C |
| 17 | 6872 | C | C | C | C | C | C | C | C |
| 18 | 6966 | A | G | G | G | A | G | G | G |
| 19 | 7036 | C | C | C | T | C | C | C | C |
| 20 | 7086 | A | A | A | A | A | A | A | A |
| 21 | 8100 | T | C | C | C | C | C | C | C |
| 22 | 8221 | G | G | G | G | G | G | G | A |
| 23 | 8677 | T | A | T | T | T | T | T | T |

(a)PS = polymorphic site;
(b)Position of PS within SEQ ID NO: 1;
(c)Alleles for haplotypes are presented 5′ to 3′ in each column;
and wherein the haplotype pair is selected from the haplotype pairs shown in the table immediately below:

| PS | PS | Haplotype Pair(c) (Part 1) | | | | | | | | | |
|---|---|---|---|---|---|---|---|---|---|---|---|
| No.(a) | Position(b) | 3/3 | 3/4 | 3/12 | 3/13 | 3/14 | 3/16 | 11/1 | 11/2 | 11/3 | 11/4 |
| 1 | 1283 | C/C | C/C | C/G | C/G | C/G | C/G | G/C | G/C | G/C | G/C |
| 2 | 1714 | C/C | C/C | C/C | C/C | C/C | C/T | C/C | C/C | C/C | C/C |
| 3 | 2051 | T/T | T/T | T/T | T/T | T/T | T/T | T/T | T/T | T/T | T/T |
| 4 | 2124 | G/G | C/C | G/G | G/G | C/C | C/T | C/C | C/C | C/C | C/C |
| 5 | 2287 | C/C | C/C | C/C | C/C | C/C | C/C | C/C | C/C | C/C | C/C |
| 6 | 2408 | A/A | A/A | A/A | A/A | A/A | A/A | A/A | A/A | A/A | A/A |
| 7 | 4768 | A/A | A/A | A/A | A/A | A/A | A/A | A/A | A/A | A/A | A/A |
| 8 | 4785 | G/G | G/G | G/G | G/G | G/G | G/G | G/A | G/G | G/G | G/G |
| 9 | 4813 | T/T | T/T | T/T | T/T | T/T | T/T | T/T | T/T | T/T | T/T |
| 10 | 4882 | A/A | A/A | A/A | A/A | A/A | A/A | A/A | A/A | A/A | A/A |
| 11 | 4976 | T/T | T/T | T/T | T/T | T/T | T/T | T/T | T/T | T/T | T/T |
| 12 | 5600 | T/T | T/T | T/C | T/C | T/T | T/C | C/T | C/C | C/T | C/T |
| 13 | 5820 | T/T | T/T | T/T | T/T | T/T | T/T | T/T | T/T | T/T | T/T |
| 14 | 6536 | T/T | T/T | T/T | T/T | T/T | T/A | T/T | T/T | T/T | T/T |
| 15 | 6607 | G/G | G/G | G/G | G/G | G/G | G/G | G/G | G/G | G/G | G/G |
| 16 | 6617 | C/C | C/C | C/C | C/T | C/C | C/C | C/C | C/C | C/C | C/C |
| 17 | 6872 | C/C | C/G | C/C | C/C | C/C | C/C | C/C | C/C | C/C | C/G |
| 18 | 6966 | G/G | G/G | G/G | G/A | G/G | G/G | G/G | G/G | G/G | G/G |
| 19 | 7036 | C/C | C/C | C/T | C/C | C/C | C/C | C/C | C/C | C/C | C/C |
| 20 | 7086 | A/A | A/G | A/A | A/A | A/A | A/A | A/A | A/A | A/A | A/G |
| 21 | 8100 | C/C | C/C | C/C | C/C | C/C | C/C | C/C | C/C | C/C | C/C |
| 22 | 8221 | G/G | G/G | G/G | G/G | G/G | G/A | G/G | G/G | G/G | G/G |
| 23 | 8677 | T/T | T/T | T/T | T/T | T/T | T/T | T/T | T/T | T/T | T/T |
| PS | PS | Haplotype Pair(c) (Part 2) | | | | | | | | | |
| No.(a) | Position(b) | 11/5 | 11/6 | 11/7 | 11/8 | 11/9 | 11/10 | 11/11 | 11/12 | 11/15 | 11/16 |
| 1 | 1283 | G/C | G/C | G/C | G/G | G/G | G/G | G/G | G/G | G/G | G/G |
| 2 | 1714 | C/C | C/C | C/C | C/C | C/C | C/C | C/C | C/C | C/T | C/T |
| 3 | 2051 | T/T | T/T | T/T | T/C | T/T | T/T | T/T | T/T | T/T | T/T |
| 4 | 2124 | C/C | C/C | C/C | C/C | C/C | C/C | C/C | C/C | C/C | C/T |
| 5 | 2287 | C/C | C/C | C/G | C/C | C/C | C/C | C/C | C/C | C/G | C/C |
| 6 | 2408 | A/A | A/G | A/A | A/A | A/A | A/A | A/A | A/A | A/A | A/A |
| 7 | 4768 | A/G | A/A | A/A | A/A | A/A | A/A | A/A | A/A | A/A | A/A |
| 8 | 4785 | G/G | G/G | G/G | G/G | G/G | G/G | G/G | G/G | G/G | G/G |
| 9 | 4813 | T/C | T/T | T/T | T/T | T/T | T/T | T/T | T/T | T/T | T/T |
| 10 | 4882 | A/C | A/G | A/A | A/A | A/A | A/A | A/A | A/A | A/A | A/A |
| 11 | 4976 | T/T | T/T | T/T | T/A | T/T | T/T | T/T | T/T | T/T | T/T |
| 12 | 5600 | C/T | C/T | C/C | C/C | C/C | C/C | C/C | C/C | C/C | C/C |
| 13 | 5820 | T/T | T/T | T/T | T/T | T/G | T/T | T/T | T/T | T/T | T/T |
| 14 | 6536 | T/T | T/T | T/A | T/T | T/T | T/T | T/T | T/T | T/A | T/A |
| 15 | 6607 | G/G | G/G | G/G | G/G | G/A | G/G | G/G | G/G | G/G | G/G |
| 16 | 6617 | C/C | C/C | C/C | C/C | C/T | C/C | C/C | C/C | C/C | C/C |
| 17 | 6872 | C/C | C/G | C/C | C/C | C/C | C/C | C/C | C/C | C/C | C/C |
| 18 | 6966 | G/G | G/G | G/G | G/G | G/A | G/G | G/G | G/G | G/G | G/G |
| 19 | 7036 | C/C | C/C | C/C | C/C | C/C | C/C | C/C | C/T | C/C | C/C |
| 20 | 7086 | A/A | A/G | A/A | A/A | A/A | A/A | A/A | A/A | A/A | A/A |
| 21 | 8100 | C/C | C/C | C/C | C/C | C/T | C/C | C/C | C/C | C/C | C/C |
| 22 | 8221 | G/G | G/G | G/G | G/G | G/G | G/G | G/G | G/G | G/G | G/A |
| 23 | 8677 | T/T | T/T | T/T | T/T | T/T | T/A | T/T | T/T | T/T | T/T |

(a)PS = polymorphic site;
(b)Position of PS in SEQ ID NO: 1;
(c)Haplotype pairs are represented as 1^{st} haplotype/2^{nd} haplotype; with alleles of each haplotype shown 5′ to 3′ as 1^{st} polymorphism/2^{nd} polymorphism in each column;
wherein a statistically significant different frequency of the haplotype or haplotype pair in the trait population than in the reference population indicates the trait is associated with the haplotype or haplotype pair.

10. A method for reducing the potential for bias in a clinical trial of a candidate drug for treating a disease or condition predicted to be associated with UCP2 activity, the method comprising determining which of the UCP2 haplotypes or UCP2 haplotype pairs shown in the tables immediately below is present in each individual that is participating in the trial; and assigning each individual to a treatment group or a control group to produce an equal number of each of the determined UCP2 haplotypes or haplotype pairs in the treatment group and the control group:

| PS | PS | Haplotype Number(c) (Part 1) | | | | | | | |
|---|---|---|---|---|---|---|---|---|---|
| No.(a) | Position(b) | 1 | 2 | 3 | 4 | 5 | 6 | 7 | 8 |
| 1 | 1283 | C | C | C | C | C | C | C | G |
| 2 | 1714 | C | C | C | C | C | C | C | C |
| 3 | 2051 | T | T | T | T | T | T | T | C |
| 4 | 2124 | C | C | C | C | C | C | C | C |
| 5 | 2287 | C | C | C | C | C | C | G | C |
| 6 | 2408 | A | A | A | A | A | G | A | A |
| 7 | 4768 | A | A | A | A | G | A | A | A |
| 8 | 4785 | A | G | G | G | G | G | G | G |
| 9 | 4813 | T | T | T | T | C | T | T | T |
| 10 | 4882 | A | A | A | A | C | A | A | A |
| 11 | 4976 | T | T | T | T | T | T | T | A |
| 12 | 5600 | T | C | T | T | T | T | C | C |
| 13 | 5820 | T | T | T | T | T | T | T | T |
| 14 | 6536 | T | T | T | T | T | T | A | T |
| 15 | 6607 | G | G | G | G | G | G | G | G |
| 16 | 6617 | C | C | C | C | C | C | C | C |
| 17 | 6872 | C | C | C | G | C | G | C | C |
| 18 | 6966 | G | G | G | G | G | G | G | G |
| 19 | 7036 | C | C | C | C | C | C | C | C |
| 20 | 7086 | A | A | A | G | A | G | A | A |
| 21 | 8100 | C | C | C | C | C | C | C | C |
| 22 | 8221 | G | G | G | G | G | G | G | G |
| 23 | 8677 | T | T | T | T | T | T | T | T |
| PS | PS | Haplotype Number(c) (Part 2) | | | | | | | |
| No.(a) | Posiition(b) | 9 | 10 | 11 | 12 | 13 | 14 | 15 | 16 |
| 1 | 1283 | G | G | G | G | G | G | G | G |
| 2 | 1714 | C | C | C | C | C | C | T | T |
| 3 | 2051 | T | T | T | T | T | T | T | T |
| 4 | 2124 | C | C | C | C | C | C | C | T |
| 5 | 2287 | C | C | C | C | C | C | G | C |
| 6 | 2408 | A | A | A | A | A | A | A | A |
| 7 | 4768 | A | A | A | A | A | A | A | A |
| 8 | 4785 | G | G | G | G | G | G | G | G |
| 9 | 4813 | T | T | T | T | T | T | T | T |
| 10 | 4882 | A | A | A | A | A | A | A | A |
| 11 | 4976 | T | T | T | T | T | T | T | T |
| 12 | 5600 | C | C | C | C | C | T | C | C |
| 13 | 5820 | G | T | T | T | T | T | T | T |
| 14 | 6536 | T | T | T | T | T | T | A | A |
| 15 | 6607 | A | G | G | G | G | G | G | G |
| 16 | 6617 | T | C | C | C | T | C | C | C |
| 17 | 6872 | C | C | C | C | C | C | C | C |
| 18 | 6966 | A | G | G | G | A | G | G | G |
| 19 | 7036 | C | C | C | T | C | C | C | C |
| 20 | 7086 | A | A | A | A | A | A | A | A |
| 21 | 8100 | T | C | C | C | C | C | C | C |
| 22 | 8221 | G | G | G | G | G | G | G | A |
| 23 | 8677 | T | A | T | T | T | T | T | T |

(a)PS = polymorphic site;
(b)Position of PS within SEQ ID NO: 1;
(c)Alleles for haplotypes are presented 5′ to 3′ in each column;

| PS | PS | | | | | | | | |
|---|---|---|---|---|---|---|---|---|---|
| No. | Posi- | Haplotype Pair(c) (Part 1) | | | | | | | |
| (a) | ition(b) | 3/3 | 3/4 | 3/12 | 3/13 | 3/14 | 3/16 | 11/1 | 11/2 |
| 1 | 1283 | C/C | C/C | C/G | C/G | C/G | C/G | G/C | G/C |
| 2 | 1714 | C/C | C/C | C/C | C/C | C/C | C/T | C/C | C/C |
| 3 | 2051 | T/T | T/T | T/T | T/T | T/T | T/T | T/T | T/T |
| 4 | 2124 | C/C | C/C | C/C | C/C | C/C | C/T | C/C | C/C |
| 5 | 2287 | C/C | C/C | C/C | C/C | C/C | C/C | C/C | C/C |
| 6 | 2408 | A/A | A/A | A/A | A/A | A/A | A/A | A/A | A/A |
| 7 | 4768 | A/A | A/A | A/A | A/A | A/A | A/A | A/A | A/A |
| 8 | 4785 | G/G | G/G | G/G | G/G | G/G | G/G | G/A | G/G |
| 9 | 4813 | T/T | T/T | T/T | T/T | T/T | T/T | T/T | T/T |
| 10 | 4882 | A/A | A/A | A/A | A/A | A/A | A/A | A/A | A/A |
| 11 | 4976 | T/T | T/T | T/T | T/T | T/T | T/T | T/T | T/T |
| 12 | 5600 | T/T | T/T | T/C | T/C | T/T | T/C | C/T | C/C |
| 13 | 5820 | T/T | T/T | T/T | T/T | T/T | T/T | T/T | T/T |
| 14 | 6536 | T/T | T/T | T/T | T/T | T/T | T/A | T/T | T/T |
| 15 | 6607 | G/G | G/G | G/G | G/G | G/G | G/G | G/G | G/G |
| 16 | 6617 | C/C | C/C | C/C | C/T | C/C | C/C | C/C | C/C |
| 17 | 6872 | C/C | G/G | C/C | C/C | C/C | C/C | C/C | C/C |
| 18 | 6966 | G/G | G/G | G/G | G/A | G/G | G/G | G/G | G/G |
| 19 | 7036 | C/C | C/C | C/T | C/C | C/C | C/C | C/C | C/C |
| 20 | 7086 | A/A | A/G | A/A | A/A | A/A | A/A | A/A | A/A |
| 21 | 8100 | G/C | C/C | C/C | C/C | C/C | C/C | C/C | C/C |
| 22 | 8221 | G/G | G/G | G/G | G/G | G/G | G/A | G/G | G/G |
| 23 | 8677 | T/T | T/T | T/T | T/T | T/T | T/T | T/T | T/T |
| PS | PS | | | | | | | | |
| No. | Posi- | Haplotype Pair(c) (Part 2) | | | | | | | |
| (a) | tion(b) | 11/3 | 11/4 | 11/5 | 11/6 | 11/7 | 11/8 | 11/9 | 11/10 |
| 1 | 1283 | G/C | G/C | G/C | G/C | G/C | G/G | G/G | G/G |
| 2 | 1714 | C/C | C/C | C/C | C/C | C/C | C/C | C/C | C/C |
| 3 | 2051 | T/T | T/T | T/T | T/T | T/T | T/C | T/T | T/T |
| 4 | 2124 | C/C | C/C | C/C | C/C | C/C | C/C | C/C | C/C |
| 5 | 2287 | C/C | C/C | C/C | C/C | G/G | C/C | C/C | C/C |
| 6 | 2408 | A/A | A/A | A/A | A/G | A/A | A/A | A/A | A/A |
| 7 | 4768 | A/A | A/A | A/G | A/A | A/A | A/A | A/A | A/A |
| 8 | 4785 | G/G | G/G | G/G | G/G | G/G | G/G | G/G | G/G |
| 9 | 4813 | T/T | T/T | T/C | T/T | T/T | T/T | T/T | T/T |
| 10 | 4882 | A/A | A/A | A/C | A/A | A/A | A/A | A/A | A/A |
| 11 | 4976 | T/T | T/T | T/T | T/T | T/T | T/A | T/T | T/T |
| 12 | 5600 | C/T | C/T | C/T | C/T | C/C | C/C | C/C | C/C |
| 13 | 5820 | T/T | T/T | T/T | T/T | T/T | T/T | T/G | T/T |
| 14 | 6536 | T/T | T/T | T/T | T/T | T/A | T/T | T/T | T/T |
| 15 | 6607 | G/G | G/G | G/G | G/G | G/G | G/G | G/A | G/G |
| 16 | 6617 | C/C | C/C | C/C | C/C | C/C | C/C | C/T | C/C |
| 17 | 6872 | C/C | C/G | C/C | G/G | C/C | C/C | C/C | C/C |
| 18 | 6966 | G/G | G/G | G/G | G/G | G/G | G/G | G/A | G/G |
| 19 | 7036 | C/C | C/C | C/C | C/C | C/C | C/C | C/C | C/C |
| 20 | 7086 | A/A | A/G | A/A | A/G | A/A | A/A | A/A | A/A |
| 21 | 8100 | C/C | C/C | C/C | C/C | C/C | C/C | C/T | C/C |
| 22 | 8221 | G/G | G/G | G/G | G/G | G/G | G/G | G/G | G/G |
| 23 | 8677 | T/T | T/T | T/T | T/T | T/T | T/T | T/T | T/A |
| | | |
|---|---|---|
| PS | PS | Haplotype Pair(c)(Part 3) |
| No.(a) | Position(b) | 11/11 | 11/12 | 11/15 | 11/16 |
|---|---|---|---|---|---|
| 1 | 1283 | G/G | G/G | G/G | G/G |
| 2 | 1714 | C/C | C/C | C/T | C/T |
| 3 | 2051 | T/T | T/T | T/T | T/T |
| 4 | 2124 | C/C | C/C | C/C | C/T |
| 5 | 2287 | C/C | C/C | C/G | C/C |
| 6 | 2408 | A/A | A/A | A/A | A/A |
| 7 | 4768 | A/A | A/A | A/A | A/A |
| 8 | 4785 | G/G | G/G | G/G | G/G |
| 9 | 4813 | T/T | T/T | T/T | T/T |
| 10 | 4882 | A/A | A/A | A/A | A/A |
| 11 | 4976 | T/T | T/T | T/T | T/T |
| 12 | 5600 | C/C | C/C | C/C | C/C |
| 13 | 5820 | T/T | T/T | T/T | T/T |
| 14 | 6536 | T/T | T/T | T/A | T/A |
| 15 | 6607 | G/G | G/G | G/G | G/G |
| 16 | 6617 | C/C | C/C | C/C | C/C |
| 17 | 6872 | C/C | C/C | C/C | C/C |
| 18 | 6966 | G/G | G/G | G/G | G/G |
| 19 | 7036 | C/C | C/T | C/C | C/C |
| 20 | 7086 | A/A | A/A | A/A | A/A |
| 21 | 8100 | C/C | C/C | C/C | C/C |
| 22 | 8221 | G/G | G/G | G/G | G/A |
| 23 | 8677 | T/T | T/T | T/T | T/T |

(a)PS polymorphic site;
(b)Position of PS in SEQ ID NO: 1;
(c)Haplotype pairs are represented as 1^{st} haplotype/2^{nd} haplotype; with alleles of each haplotype shown 5′ to 3′ as 1^{st} polymorphism/2^{nd} polymorphism in each column.

11. An isolated polynucleotide comprising a nucleotide sequence selected from the group consisting of:
(a) a first nucleotide sequence which comprises an uncoupling protein 2 (mitochondrial, proton carrier) (UCP2) isogene, wherein the UCP2 isogene is selected from the group consisting of isogenes 1 and 3-16 shown in the table immediately below and wherein each of the isogenes comprises the regions of SEQ ID NO:1 shown in the table immediately below, except where substituted by the corresponding sequence of polymorphisms whose positions and alleles are set forth in the table immediately below; and
(b) a second nucleotide sequence which is complementary to the first nucleotide sequence

| Region | PS | PS | Isogene Number(d) | | | | | | |
|---|---|---|---|---|---|---|---|---|---|
| Examined(a) | No.(b) | Position(c) | 1 | 3 | 4 | 5 | 6 | 7 | 8 |
| 1000-2531 | 1 | 1283 | C | C | C | C | C | C | G |
| 1000-2531 | 2 | 1714 | C | C | C | C | C | C | C |
| 1000-2531 | 3 | 2051 | T | T | T | T | T | T | C |
| 1000-2531 | 4 | 2124 | C | C | C | C | C | C | C |
| 1000-2531 | 5 | 2287 | C | C | C | C | C | G | C |
| 1000-2531 | 6 | 2408 | A | A | A | A | G | A | A |
| 4393-5236 | 7 | 4768 | A | A | A | G | A | A | A |
| 4393-5236 | 8 | 4785 | A | G | G | G | G | G | G |
| 4393-5236 | 9 | 4813 | T | T | T | C | T | T | T |
| 4393-5236 | 10 | 4882 | A | A | A | C | A | A | A |
| 4393-5236 | 11 | 4976 | T | T | T | T | T | T | A |
| 5399-5908 | 12 | 5600 | T | T | T | T | T | C | C |
| 5399-5908 | 13 | 5820 | T | T | T | T | T | T | T |
| 6413-7225 | 14 | 6536 | T | T | T | T | T | A | T |
| 6413-7225 | 15 | 6607 | G | G | G | G | G | G | G |
| 6413-7225 | 16 | 6617 | C | C | C | C | C | C | C |
| 6413-7225 | 17 | 6872 | C | C | G | C | G | C | C |
| 6413-7225 | 18 | 6966 | G | G | G | G | G | G | G |
| 6413-7225 | 19 | 7036 | C | C | C | C | C | C | C |
| 6413-7225 | 20 | 7086 | A | A | G | A | G | A | A |
| 7764-8311 | 21 | 8100 | C | C | C | C | C | C | C |
| 7764-8311 | 22 | 8221 | G | G | G | G | G | G | G |
| 8367-8792 | 23 | 8677 | T | T | T | T | T | T | T |
| Region | PS | PS | Isogene Number(d) | | | | | | | |
|---|---|---|---|---|---|---|---|---|---|---|
| Examined(a) | No.(b) | tion(c) | 9 | 10 | 11 | 12 | 13 | 14 | 15 | 16 |
| 1000-2531 | 1 | 1283 | G | G | G | G | G | G | C | C |
| 1000-2531 | 2 | 1714 | C | C | C | C | C | C | T | T |
| 1000-2531 | 3 | 2051 | T | T | T | T | T | T | T | T |
| 1000-2531 | 4 | 2124 | C | C | C | C | C | C | C | T |
| 1000-2531 | 5 | 2287 | C | C | C | C | C | C | G | C |
| 1000-2531 | 6 | 2408 | A | A | A | A | A | A | A | A |
| 4393-5236 | 7 | 4768 | A | A | A | A | A | A | A | A |
| 4393-5236 | 8 | 4785 | G | G | G | G | G | G | C | G |
| 4393-5236 | 9 | 4813 | T | T | T | T | T | T | T | T |
| 4393-5236 | 10 | 4882 | A | A | A | A | A | A | A | A |
| 4393-5236 | 11 | 4976 | T | T | T | T | T | T | T | T |
| 5399-5908 | 12 | 5600 | C | C | C | C | C | T | C | C |
| 5399-5908 | 13 | 5820 | G | T | T | T | T | T | T | T |
| 6413-7225 | 14 | 6536 | T | T | T | T | T | T | A | A |
| 6413-7225 | 15 | 6607 | A | G | G | G | G | G | G | G |
| 6413-7225 | 16 | 6617 | T | C | C | C | T | C | C | C |
| 6413-7225 | 17 | 6872 | C | C | C | C | C | C | C | C |
| 6413-7225 | 18 | 6966 | A | G | G | G | A | G | G | G |
| 6413-7225 | 19 | 7036 | C | C | C | T | C | C | C | C |
| 6413-7225 | 20 | 7086 | A | A | A | A | A | A | A | A |
| 7764-8311 | 21 | 8100 | T | C | C | C | C | C | C | C |
| 7764-8311 | 22 | 8221 | G | G | G | G | G | G | G | A |
| 8367-8792 | 23 | 8677 | T | A | T | T | T | T | T | T |

(a)Region examined represents the nucleotide positions defining the start and stop positions within SEQ ID NO: 1 of each sequenced region;
(b)PS = polymorphic site;
(c)Position of PS in SEQ ID NO: 1;
(d)Alleles for isogenes are presented 5′ to 3′ in each column.

12. A recombinant nonhuman organism transformed or transfected with the isolated polynucleotide of claim 11, wherein the organism expresses an UCP2 protein that is encoded by the sequence of the isolated polynucleotide.

13. An isolated fragment of an uncoupling protein 2 (mitochondrial, proton carrier) (UCP2) isogene, wherein the fragment comprises at least 50 nucleotides in one of the regions of SEQ ID NO:1 shown in the table immediately below and wherein the fragment comprises one or more polymorphisms selected from the group consisting of guanine at PS1, thymine at PS2, cytosine at PS3, thymine at PS4, guanine at PS5, guanine at PS6, guanine at PS7, adenine at PS8, cytosine at PS9, cytosine at PS10, adenine at PS11, guanine at PS13, adenine at PS14, adenine at PS15, thymine at PS16, guanine at PS17, adenine at PS18, thymine at PS19, guanine at PS20, thymine at PS21, adenine at PS22 and adenine at PS23, wherein the selected polymorphism has the position set forth in the table immediately below:

| Region | PS | PS | Isogene Number(d) | | | | | | |
|---|---|---|---|---|---|---|---|---|---|
| Examined(a) | No.(b) | Position(c) | 1 | 3 | 4 | 5 | 6 | 7 | 8 |
| 1000-2531 | 1 | 1283 | C | C | C | C | C | C | G |
| 1000-2531 | 2 | 1714 | C | C | C | C | C | C | C |
| 1000-2531 | 3 | 2051 | T | T | T | T | T | T | C |
| 1000-2531 | 4 | 2124 | C | C | C | C | C | C | C |
| 1000-2531 | 5 | 2287 | C | C | C | C | C | G | C |
| 1000-2531 | 6 | 2408 | A | A | A | A | G | A | A |
| 4393-5236 | 7 | 4768 | A | A | A | G | A | A | A |
| 4393-5236 | 8 | 4785 | A | G | G | G | G | G | G |
| 4393-5236 | 9 | 4813 | T | T | T | C | T | T | T |
| 4393-5236 | 10 | 4882 | A | A | A | C | A | A | A |
| 4393-5236 | 11 | 4976 | T | T | T | T | T | T | A |
| 5399-5908 | 12 | 5600 | T | T | T | T | T | C | C |
| 5399-5908 | 13 | 5820 | T | T | T | T | T | T | T |
| 6413-7225 | 14 | 6536 | T | T | T | T | T | A | T |
| 6413-7225 | 15 | 6607 | G | G | G | G | G | G | G |
| 6413-7225 | 16 | 6617 | C | C | C | C | C | C | C |
| 6413-7225 | 17 | 6872 | C | C | G | C | G | C | C |
| 6413-7225 | 18 | 6966 | G | C | G | G | G | G | G |
| 6413-7225 | 19 | 7036 | C | C | C | C | C | C | C |
| 6413-7225 | 20 | 7086 | A | A | G | A | G | A | A |
| 7764-8311 | 21 | 8100 | C | C | C | C | C | C | C |
| 7764-8311 | 22 | 8221 | G | G | G | G | G | G | G |
| 8367-8792 | 23 | 8677 | T | T | T | T | T | T | T |
| Region | PS | PS | Isogene Number(d) | | | | | | | |
|---|---|---|---|---|---|---|---|---|---|---|
| Examined(a) | No.(b) | Position(c) | 9 | 10 | 11 | 12 | 13 | 14 | 15 | 16 |
| 1000-2531 | 1 | 1283 | G | G | G | G | G | G | G | G |
| 1000-2531 | 2 | 1714 | C | C | C | C | C | C | T | T |
| 1000-2531 | 3 | 2051 | T | T | T | T | T | T | T | T |
| 1000-2531 | 4 | 2124 | C | C | C | C | C | C | C | T |
| 1000-2531 | 5 | 2287 | C | C | C | C | C | C | G | C |
| 1000-2531 | 6 | 2408 | A | A | A | A | A | A | A | A |
| 4393-5236 | 7 | 4768 | A | A | A | A | A | A | A | A |
| 4393-5236 | 8 | 4785 | G | G | G | G | G | G | G | G |
| 4393-5236 | 9 | 4813 | T | T | T | T | T | T | T | T |
| 4393-5236 | 10 | 4882 | A | A | A | A | A | A | A | A |
| 4393-5236 | 11 | 4976 | T | T | T | T | T | T | T | T |
| 5399-5908 | 12 | 5600 | C | C | C | C | C | T | C | C |
| 5399-5908 | 13 | 5820 | G | T | T | T | T | T | T | T |
| 6413-7225 | 14 | 6536 | T | T | T | T | T | T | A | A |
| 6413-7225 | 15 | 6607 | A | G | G | G | G | G | G | G |
| 6413-7225 | 16 | 6617 | T | C | C | C | T | C | C | C |
| 6413-7225 | 17 | 6872 | C | C | C | C | C | C | C | C |
| 6413-7225 | 18 | 6966 | A | G | G | G | A | G | G | G |
| 6413-7225 | 19 | 7036 | C | C | C | T | C | C | C | C |
| 6413-7225 | 20 | 7086 | A | A | A | A | A | A | A | A |
| 7764-8311 | 21 | 8100 | T | C | C | C | C | C | C | C |
| 7764-8311 | 22 | 8221 | G | G | G | G | G | G | G | A |
| 8367-8792 | 23 | 8677 | T | A | T | T | T | T | T | T |

(a)Region examined represents the nucleotide positions defining the start and stop positions within SEQ ID NO: 1 of the regions sequenced;
(b)PS polymorphic site; (c) Position of PS within SEQ ID NO: 1;
(d)Alleles for UCP2 isogenes are presented 5′ to 3′ in each column.

14. An isolated polynucleotide comprising a nucleotide sequence selected from the group consisting of:
(a) a first nucleotide sequence which comprises a coding sequence variant for an UCP2 isogene, wherein the coding sequence variant is selected from the group consisting of A, B and C represented in the table below and wherein the selected coding sequence variant comprises the regions of SEQ ID NO:2 shown in the table below, except where substituted by the corresponding sequence of polymorphisms whose positions and alleles are set forth in the table immediately below; and
(b) a second nucleotide sequence which is complementary to the first nucleotide sequence

| Region | PS | PS | Coding Sequence Variants(d) | | |
|---|---|---|---|---|---|
| Examined(a) | No.(b) | Position(c) | A | B | C |
| 119-930 | 12 | 164 | T | C | C |
| 119-930 | 18 | 582 | G | A | A |
| 119-930 | 21 | 750 | C | T | C |

(a)Region examined represents the nucleotide positions defining the start and stop positions within SEQ ID NO: 2 of the regions sequenced;
(b)PS = polymorphic site;
(c)Position of PS in SEQ ID NO: 2;
(d)Alleles for the coding sequence variants are presented 5′ to 3′ in each column.

15. A recombinant nonhuman organism transformed or transfected with the isolated polynucleotide of claim 14, wherein the organism expresses an uncoupling protein 2 (mitochondrial, proton carrier) (UCP2) protein that is encoded by the coding sequence variant.

16. An isolated fragment of an UCP2 coding sequence, wherein the fragment comprises at least 50 nucleotides and one or more polymorphisms selected from the group consisting of adenine at a position corresponding to nucleotide 582 and thymine at a position corresponding to nucleotide 750 in SEQ ID NO:2.

17. A method for screening for compounds targeting the UCP2 protein to treat a condition or disease predicted to be associated with UCP2 activity, the method comprising:
(a) determining the frequency of each of the UCP2 haplotypes shown in the table immediately below in a population having the disease; and
(b) if the frequency of the UCP2 haplotype meets a desired cutoff frequency criterion, then screening for a compound that displays a desired agonist or antagonist activity for the UCP2 isoform defined by that haplotype:

| PS | PS | Haplotype Number(c) (Part 1) | | | | | | | |
|---|---|---|---|---|---|---|---|---|---|
| No.(a) | Position(b) | 1 | 2 | 3 | 4 | 5 | 6 | 7 | 8 |
| 1 | 1283 | C | C | C | C | C | C | C | G |
| 2 | 1714 | C | C | C | C | C | C | C | C |
| 3 | 2051 | T | T | T | T | T | T | T | C |
| 4 | 2124 | C | C | C | C | C | C | C | C |
| 5 | 2287 | C | C | C | C | C | C | G | C |
| 6 | 2408 | A | A | A | A | A | G | A | A |
| 7 | 4768 | A | A | A | A | G | A | A | A |
| 8 | 4785 | A | G | G | G | G | G | G | G |
| 9 | 4813 | T | T | T | T | C | T | T | T |
| 10 | 4882 | A | A | A | A | C | A | A | A |
| 11 | 4976 | T | T | T | T | T | T | T | A |
| 12 | 5600 | T | C | T | T | T | T | C | C |
| 13 | 5820 | T | T | T | T | T | T | T | T |
| 14 | 6536 | T | T | T | T | T | T | A | T |
| 15 | 6607 | G | G | G | G | G | G | G | G |
| 16 | 6617 | C | C | C | C | C | C | C | C |
| 17 | 6872 | C | C | C | C | C | C | C | C |
| 18 | 6966 | G | G | G | G | G | G | G | G |
| 19 | 7036 | C | C | C | C | C | C | C | C |
| 20 | 7086 | A | A | A | G | A | G | A | A |
| 21 | 8100 | C | C | C | C | C | C | C | C |
| 22 | 8221 | G | G | G | G | G | G | G | G |
| 23 | 8677 | T | T | T | T | T | T | T | T |
| PS | PS | Haplotype Number(c) (Part 2) | | | | | | | |
| No.(a) | Position(b) | 9 | 10 | 11 | 12 | 13 | 14 | 15 | 16 |
| 1 | 1283 | G | G | G | G | G | G | G | G |
| 2 | 1714 | C | C | C | C | C | C | T | T |
| 3 | 2051 | T | T | T | T | T | T | T | T |
| 4 | 2124 | C | C | C | C | C | C | C | T |
| 5 | 2287 | C | C | C | C | C | C | G | C |
| 6 | 2408 | A | A | A | A | A | A | A | A |
| 7 | 4768 | A | A | A | A | A | A | A | A |
| 8 | 4785 | G | G | G | G | G | G | G | G |
| 9 | 4813 | T | T | T | T | T | T | T | T |
| 10 | 4882 | A | A | A | A | A | A | A | A |
| 11 | 4976 | T | T | T | T | T | T | T | T |
| 12 | 5600 | C | C | C | C | C | T | C | C |
| 13 | 5820 | G | T | T | T | T | T | T | T |
| 14 | 6536 | T | T | T | T | T | T | A | A |
| 15 | 6607 | A | G | G | G | G | G | G | G |
| 16 | 6617 | T | C | C | C | T | C | C | C |
| 17 | 6872 | C | C | C | C | C | C | C | C |
| 18 | 6966 | A | G | G | G | A | G | G | G |
| 19 | 7036 | C | C | C | T | C | C | C | C |
| 20 | 7086 | A | A | A | A | A | A | A | A |
| 21 | 8100 | T | C | C | C | C | C | C | C |
| 22 | 8221 | G | G | G | G | G | G | G | A |
| 23 | 8677 | T | A | T | T | T | T | T | T |

(a)PS = polymorphic site;
(b)Position of PS within SEQ ID NO: 1;
(c)Alleles for haplotypes are presented 5′ to 3′ in each column.

18. A method for validating the UCP2 protein as a candidate target for treating a medical condition predicted to be associated with UCP2 activity, the method comprising:
(a) comparing the frequency of each of the UCP2 haplotypes in the table shown immediately below between first and second populations, wherein the first population is a group of individuals having the medical condition and the second population is a group of individuals lacking the medical condition; and
(b) making a decision whether to pursue UCP2 as a target for treating the medical condition; wherein if at least one of the UCP2 haplotypes is present in a frequency in the first population that is different from the frequency in the second population at a statistically significant level, then the decision is to pursue the UCP2 protein as a target and if none of the UCP2 haplotypes are seen in a different frequency, at a statistically significant level, between the first and second populations, then the decision is to not pursue the UCP2 protein as a target

| PS | PS | Haplotype Number(c) (Part 1) | | | | | | | |
|---|---|---|---|---|---|---|---|---|---|
| No.(a) | Position(b) | 1 | 2 | 3 | 4 | 5 | 6 | 7 | 8 |
| 1 | 1283 | C | C | C | C | C | C | C | G |
| 2 | 1714 | C | C | C | C | C | C | C | C |
| 3 | 2051 | T | T | T | T | T | T | T | C |
| 4 | 2124 | C | C | C | C | C | C | C | C |
| 5 | 2287 | C | C | C | C | C | C | G | C |
| 6 | 2408 | A | A | A | A | A | G | A | A |
| 7 | 4768 | A | A | A | A | G | A | A | A |
| 8 | 4785 | A | G | G | G | G | G | G | G |
| 9 | 4813 | T | T | T | T | C | T | T | T |
| 10 | 4882 | A | A | A | A | C | A | A | A |
| 11 | 4976 | T | T | T | T | T | T | T | A |
| 12 | 5600 | T | C | T | T | T | T | C | C |
| 13 | 5820 | T | T | T | T | T | T | T | T |
| 14 | 6536 | T | T | T | T | T | T | A | T |
| 15 | 6607 | G | G | G | G | G | G | G | G |
| 16 | 6617 | C | C | C | C | C | C | C | C |
| 17 | 6872 | C | C | C | G | C | G | C | C |
| 18 | 6966 | G | G | G | G | G | G | G | G |
| 19 | 7036 | C | C | C | C | C | C | C | C |
| 20 | 7086 | A | A | A | G | A | G | A | A |
| 21 | 8100 | C | C | C | C | C | C | C | C |
| 22 | 8221 | G | G | G | G | G | G | G | G |
| 23 | 8677 | T | T | T | T | T | T | T | T |
| PS | PS | Haplotype Number(c) (Part 2) | | | | | | | |
| No.(a) | Position(b) | 9 | 10 | 11 | 12 | 13 | 14 | 15 | 16 |
| 1 | 1283 | G | G | G | G | G | G | G | G |
| 2 | 1714 | C | C | C | C | C | C | T | T |
| 3 | 2051 | T | T | T | T | T | T | T | T |
| 4 | 2124 | C | C | C | C | C | C | C | T |
| 5 | 2287 | C | C | C | C | C | C | G | C |
| 6 | 2408 | A | A | A | A | A | A | A | A |
| 7 | 4768 | A | A | A | A | A | A | A | A |
| 8 | 4785 | G | G | G | G | G | G | G | G |
| 9 | 4813 | T | T | T | T | T | T | T | T |
| 10 | 4882 | A | A | A | A | A | A | A | A |
| 11 | 4976 | T | T | T | T | T | T | T | T |
| 12 | 5600 | C | C | C | C | C | T | C | C |
| 13 | 5820 | G | T | T | T | T | T | T | T |
| 14 | 6536 | T | T | T | T | T | T | A | A |
| 15 | 6607 | A | G | G | G | G | G | G | G |
| 16 | 6617 | T | C | C | C | T | C | C | C |
| 17 | 6872 | C | C | C | C | C | C | C | C |
| 18 | 6966 | A | G | G | G | A | G | G | G |
| 19 | 7036 | C | C | C | T | C | C | C | C |
| 20 | 7086 | A | A | A | A | A | A | A | A |
| 21 | 8100 | T | C | C | C | C | C | C | C |
| 22 | 8221 | G | G | G | G | G | G | G | A |
| 23 | 8677 | T | A | T | T | T | T | T | T |

(a)PS = polymorphic site;
(b)Position of PS within SEQ ID NO: 1;
(c)Alleles for haplotypes are presented 5′ to 3′ in each column.

19. An isolated oligonucleotide designed for detecting a polymorphism in the uncoupling protein 2 (mitochondrial, proton carrier) (UCP2) gene at a polymorphic site (PS) selected from the group consisting of PS1, PS2, PS3, PS4, PS5, PS6, PS7, PS8, PS9, PS10, PS11, PS13, PS14, PS15, PS16, PS17, PS18, PS19, PS20, PS21, PS22 and PS23, wherein the oligonucleotide contains or is located one to several nucleotides downstream of the selected PS, wherein the oligonucleotide has a length of 15 to 100 nucleotides, and wherein the selected PS has the position and alternative alleles shown in SEQ ID NO:1.

20. The isolated oligonucleotide of claim 19, which is an allele-specific oligonucleotide that specifically hybridizes to an allele of the UCP2 gene at a region containing the polymorphic site.

21. The allele-specific oligonucleotide of claim 20, which comprises a nucleotide sequence selected from the group consisting of SEQ ID NOS:4-25, the complements of SEQ ID NOS:4-25, and SEQ ID NOS:26-69.

22. The isolated oligonucleotide of claim 19, which is a primer-extension oligonucleotide.

23. The primer-extension oligonucleotide of claim 22, which comprises a nucleotide sequence selected from the group consisting of SEQ ID NOS:70-113.

24. A kit for haplotyping or genotyping the uncoupling protein 2 (mitochondrial, proton carrier) (UCP2) gene of an individual, which comprises a set of oligonucleotides designed to haplotype or genotype each of polymorphic sites (PS) PS1, PS2, PS3, PS4, PS5, PS6, PS7, PS8, PS9, PS10, PS11, PS13, PS14, PS15, PS16, PS17, PS18, PS19, PS20, PS21, PS22 and PS23, wherein the selected PS have the position and alternative alleles shown in SEQ ID NO:1.

25. The kit of claim 24, which further comprises oligonucleotides designed to genotype or haplotype PS12, wherein the selected PS has the position and alternative alleles shown in SEQ ID NO:1.

26. A genome anthology for the uncoupling protein 2 (mitochondrial, proton carrier) (UCP2) gene which comprises two or more UCP2 isogenes selected from the group consisting of isogenes 1-16 shown in the table immediately below, and wherein each of the isogenes comprises the regions of SEQ ID NO:1 shown in the table immediately below and wherein each of the isogenes 1-16 is further defined by the corresponding sequence of polymorphisms whose positions and alleles are set forth in the table immediately below:

| Region | PS | PS | Isogene Number(d) | | | | | | | |
|---|---|---|---|---|---|---|---|---|---|---|
| Examined(a) | No.(b) | Position(c) | 1 | 2 | 3 | 4 | 5 | 6 | 7 | 8 |
| 1000-2531 | 1 | 1283 | C | C | C | C | C | C | C | G |
| 1000-2531 | 2 | 1714 | C | C | C | C | C | C | C | C |
| 1000-2531 | 3 | 2051 | T | T | T | T | T | T | T | C |
| 1000-2531 | 4 | 2124 | C | C | C | C | C | C | C | C |
| 1000-2531 | 5 | 2287 | C | C | C | C | C | C | G | C |
| 1000-2531 | 6 | 2408 | A | A | A | A | A | G | A | A |
| 4393-5236 | 7 | 4768 | A | A | A | A | G | A | A | A |
| 4393-5236 | 8 | 4785 | A | G | G | G | G | G | G | G |
| 4393-5236 | 9 | 4813 | T | T | T | T | C | T | T | T |
| 4393-5236 | 10 | 4882 | A | A | A | A | C | A | A | A |
| 4393-5236 | 11 | 4976 | T | T | T | T | T | T | T | A |
| 5399-5908 | 12 | 5600 | T | C | T | T | T | T | C | C |
| 5399-5908 | 13 | 5820 | T | T | T | T | T | T | T | T |
| 6413-7225 | 14 | 6536 | T | T | T | T | T | T | A | T |
| 6413-7225 | 15 | 6607 | G | G | G | G | G | G | G | G |
| 6413-7225 | 16 | 6617 | C | C | C | C | C | C | C | C |
| 6413-7225 | 17 | 6872 | C | C | C | G | C | G | C | C |
| 6413-7225 | 18 | 6966 | G | G | G | G | G | G | G | G |
| 6413-7225 | 19 | 7036 | C | C | C | C | C | C | C | C |
| 6413-7225 | 20 | 7086 | A | A | A | G | A | G | A | A |
| 7764-8311 | 21 | 8100 | C | C | C | C | C | C | C | C |
| 7764-8311 | 22 | 8221 | G | G | G | G | G | G | G | G |
| 8367-8792 | 23 | 8677 | T | T | T | T | T | T | T | T |
| Region | PS | PS | Isogene Number(d) | | | | | | | |
| Examined(a) | No.(b) | Position(c) | 9 | 10 | 11 | 12 | 13 | 14 | 15 | 16 |
| 1000-2531 | 1 | 1283 | G | G | G | G | G | G | G | G |
| 1000-2531 | 2 | 1714 | C | C | C | C | C | C | T | T |
| 1000-2531 | 3 | 2051 | T | T | T | T | T | T | T | T |
| 1000-2531 | 4 | 2124 | C | C | C | C | C | C | C | T |
| 1000-2531 | 5 | 2287 | C | C | C | C | C | C | G | C |
| 1000-2531 | 6 | 2408 | A | A | A | A | A | A | A | A |
| 4393-5236 | 7 | 4768 | A | A | A | A | A | A | A | A |
| 4393-5236 | 8 | 4785 | G | G | G | G | G | G | G | G |
| 4393-5236 | 9 | 4813 | T | T | T | T | T | T | T | T |
| 4393-5236 | 10 | 4882 | A | A | A | A | A | A | A | A |
| 4393-5236 | 11 | 4976 | T | T | T | T | T | T | T | T |
| 5399-5908 | 12 | 5600 | C | C | C | C | C | T | C | C |
| 5399-5908 | 13 | 5820 | G | T | T | T | T | T | T | T |
| 6413-7225 | 14 | 6536 | T | T | T | T | T | T | A | A |
| 6413-7225 | 15 | 6607 | A | G | G | G | G | G | G | G |
| 6413-7225 | 16 | 6617 | T | C | C | C | T | C | C | C |
| 6413-7225 | 17 | 6872 | C | C | C | C | C | C | C | C |
| 6413-7225 | 18 | 6966 | A | G | G | G | A | G | G | G |
| 6413-7225 | 19 | 7036 | C | C | C | T | C | C | C | C |
| 6413-7225 | 20 | 7086 | A | A | A | A | A | A | A | A |
| 7764-8311 | 21 | 8100 | T | C | C | C | C | C | C | C |
| 7764-8311 | 22 | 8221 | G | G | C | G | G | G | G | A |
| 8367-8792 | 23 | 8677 | T | A | T | T | T | T | T | T |

(a)Region examined represents the nucleotide positions defining the start and stop positions within SEQ ID NO: 1 of the regions sequenced;
(b)PS = polymorphic site;
(c)Position of PS within SEQ ID NO: 1;
(d)Alleles for UCP2 isogenes are presented 5′ to 3′ in each column.


SEQUENCE LISTING (116 sequences)

SEQ ID NO: 1 - DNA, 9314 bp, Homo sapiens
  allele (1283)..(1283): PS1 polymorphic base cytosine or guanine
ggtccatctcctgacgccttttctcatcccagggctggacaggcagctggcctgggcccggctctgccttgtcacgtgcgggggccggcccgtttgcttgtctgtgtgtaggagcgtgaggtcacgctgggtgctcccgccccgccggggcctttagtgtccctggtccctaaacgccaggccgctccaccgggggagaaggcgcgaaccccagccgagcccaacggctgttgtcggttgccgggccacctgttgctgcagttctgattggttccttcccccgacaacgcggcggctgtaaccaatcgacagcgaggccggtcgcgaggccccagtcccgccctgcaggagccagccgcgcgctcgctcgcaggagggtgggtagtttgcccagcgtagggggctgggcccataaaagaggaagtgcacttaagacacggcccagtggacgctgttagaaaccgtcctggctgggaaggcaagaggtgtgtgactggacaagacttgtttctggcggtcagtcttgccatcctcacagaggttggcggcccgagagagtgtgaggcagaggcggggagtggcaagggagtgaccatctcggggaacgaaggagtaaacgcggtgatgggacgcacggaaacgggagtggagaaagtcatggagagaaccctaggcggggcggtccccgcggaaaggcggctgctccagggtctccgcacccaagtaggagctggcaggcccggccccgccccgcaggccccaccccgggccccgcccccgaggcttaagccgcgccgccgcctgcgcggagccccactgcgaagcccagctgcgcgcgccttgggattgactgtccacgctcgcccggctcgtccgacgcgccctccgccagccgacagacacagccgcacgcactgccgtgttctccctgcggctcggtgagcctggccccagccctgcgccctttgcgccccccacgcttgttatgcgtgcgctgcccgctcttccatttaccttctctcccacccaagtttgtactcttttctttctctcggttttattttttgtttttgtttgtttgtttgagacaggctttcgctctgtctcccaggctggagtgcagtggcgcgatctcggctcactgcagcctccacctcccaggttcaagcgatccgcctgccgagtagctgggattacaggcgcccgccaccacgcctggctaatttttgtgttttgtagagatggggtttcgccatgttggccaggctggcctcgaactgctsagctcaagcaatccgcccgcctcggcctcacaaagtcctagaattttaggcatgagcctccgggtccggcctgtgctaatcctttctgtccttggttctttatttctcttctctctttttcttagtcccttttgttctttccctctcccgttcagttggctgtcgtttgagcctccaccttttcactccctcctttccaccacgatgccgagccctgccttggatggggaccatcagcgatgaccacaatgacctctcccttaccaggcagctccaggcagtgttcctgcaccgcctttcccagggcttgggggctttttctagtgggctttgagctgctcaatctggcctctgcagggccggctcccagcccttccaacctcctcacagcccgacctgggacctagccaattcccggagagtctctgtcycatcgtgaccccctcacaactctcccactcaccaaagtctgatgactgtgctagggggtgcttatatagagtactgagtgttacaaaagcagaagtctggatgagaaccaatttgtgatattaagcaggtggggtgggggtggggagtgtacctaggttcattttccgccctgcttttcccctttccagtgtgtgcacttaaccagtccctgggccctgttccccatccccctccaaggcatggattgggtgggcttgtgtgtcttggggcaggtggccctttctaaactctctgcctttgctcacccacaggacacatagtatgaccattaggtgyttcgtctcccacccattttctatggaaaaccaaggggatcgggccatgatagccactggcagctttgaagaaygggacacctttagagaagcttgatcttggaggcctcaccgtgagaccttacaaagccgggtaagagtccagtccaaggaagaggtctcttgctgcctcctaaccctgtggtctaggggcaggagtcagcagggcattaacaaaaataattaccatccccaccsccgacagtgaagtggctctttccagttcacagagcactctcacacctccccgctctcattctggcccttcagctgactcggacaagccaaggatcttggtccccattttataaaggagaaractgaggcccacgtgtaacagtgattggccccaagtcatcccgggagccagcagaagagctaggacaggaacctattgttctaacttcatattgatgctagcttttgactatccctgaaaccgagattggtaatcagcccggctctgaaactggttatttgctggggactgtaaaataggattaactatttctagtcctgcattttaattgctgttagtagggccatcttacccaccctctgaaggacctgacttggcaagcccaaggcaacattcagaatatggcagctgaacctctgtgcacttgtctttgggcagcagctgggtcttattcttctctggccttcacaacatcctgcaacccagctcaaggtcaggaatgtgacagactcatgtcatcatatctctgatgcccagagaagggataccatttgcctgagccttctcagtactgtttaatcagcctgtgagaactttccttgtgaaaggccctgtctgtgcctggggctgataaaacagcaagaacgaactgaggagctgggcagcagtgcaaagcaaatactaccagctttggtgcctgtaagtgtggctcttactcatctcacatggaaataagggcagccaccttgcagggctgctctgaggattgagctaatacagtgccctgggcgttggggtggggaaagttgtggagcacctcctgggggaagggggtgtcagagcagggaatctggggagtccgagggcaccttcatcaacccaatctgtcatttgagcaccagtcttcactgagcctcgtgggcaagctggagggaaacaggaataaggtcaggccctgttctataggtcccagtgtagttgctatggtgagtatcttcatttccctgcttgccccagccacctggagtgagaagcccaagagtaagttgggtgagctgtttgtttccatgggtctctgtgttcacaaataactcccttcaccaaccagccctttcacctgagccccagcaacaaagacagtcaggcggggctcaaagcagctgctccaatgaagtcaaagaaataagctcaggggaagaagcaggtcaccctcccccactagggtgctgggctcacttcctcctggggcagtggaggagggtgtggttccaactcagaacaaaatggggcttttggtttactttatcactcttcacagctctgacctggacccctcatccctgcctgtcttgtggtgtaagtgcggatccccctaagttggaggaaaggaaactggcccaaacaaaaaggagagcagttttctctgcatcacatggtaggccaggaggagtctaatgccccagagtttactctcagcccccaaaatcacctagctaaatgttaccttatctaagaagtccttaggttttttggggttttttttttttttttttgagacaaggtctcactctctcacccagactggagcacagtggcacaatcacagctcactgcagcctcaacctcctgggctcaagcaatcgtcccaagtagctgggactataggcctgcaccaccatgtccagctaatttatttttatttatattttttagacagggtctcattatgttgccctggctggtcttgaactcctgggttcaagcagtcctcccacctctgcctcccaaagtgctaggtttttttttgtttgtttgtctgttttttgaaacagagtcttgctctgtcgcctaggctggagtgcagtggcacgatctcagctactgcaacctccacctcctgggttcaagtgattctcctgcctcagcctcctaagtagttgggaatacaggcgtgtgccaacacacccagctcatttttgtatttttagcggagatggggttttgccatgttggccaagctggtctcaaactcctgacctcaggtgattcgcccgcctcagcctcccaaagtgctgggtttacaggcgtgagccaccacacccagcccaagaagtcttttctgatcacccactcttccttctctcccaatggcattagttgttccctcctttgcattttgagagtatgtcctgtaagccccaaatgcagcttgaatcatctgcccatccaccccctgtgcccaacagtaagcctcctctagagtagatactatctcctgcatctcagtgaaccactgcccagcaaagcagtcttgctaaaacaatgactctagagatcctaagctgtgtgagagctggaggagagaattagactgatggtctgggaagggattgaattagtcatcttgtaccttttcttcttgacttaagttccagacctgtagcaaccattcctgcttagacatccagaacataagcctrtgggtctgtgcctgttrggtcttagtctgggtgaaacttttctcyacttctgtcagctctccagatgaaccacagaagcaggaatgtgggcatcatcagtgaaatctctgcatmcagcagacaaagggctggtccagtggctgtttatgaggcagcgctaggagagctctgatccagactctccctgcagtgaaagggagggagcccwtcatgaagtattgactgcttgagcaggaattgcttcaccagcacctaactgagtgcctctcgagctcacatcggttttccctcatgaggccacttggagtcttgctgagggacttggttctattagggaaggtgagtttggggatggtgagcagggagggcctggggacattgtggctaatggggcttttctcctcttggcttagattccggcagagttcctctatctcgtcttgttgctgattaaaggtgcccctgtctccagtttttctccatctcctgggacgtagcaggaaatcagcatcatggttgggttcaaggccacagatgtgccccctactgccactgtgaagtttcttggggctggcacagctgcctgcatcgcagatctcatcacctttcctctggatactgctaaagtccggttacaggtgaggggatgaagcctgggagtcttgatggtgtctactctgttccctccccaaagacacagacccctcaagggccagtgtttggagcatcgagatgactggaggtgggaagggcaacatgcttatccctgtagctaccctgtcttggccttgcagatccaaggagaaagtcaggggccagtgcgcgctacagycagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcgtgagtatggagcaagggtgtaggccccttggcccttttttctcagkgatgattgatcttagttcattcagccatatagttttttaggccccacgatccctaggaagatcaggggaacagagaactggaaggggccctggtcctccacatagttcctaagcacctgggctataccaggctctgagcagggcgtcatcccatcacagtcttcaacaccaccttgggagtaggtagtatcatcccagtgttatagaagaagagactgaggtgggaaggcagtgggtagagtggggacttggccaggggcacacagtagagagccagaaaacacacagtagagagccaggacactcgtctctaaggccagcgttcttccctttcacctccttagtatgccatgccaaccctccattttacacatgacgaaacagagccccagacaaaaggttgtctttcccagatcacatggcaggaagaagtaaagctgacctgagatcccaagtcttaggaatcccagtcctcagaaagccacttctctctgagccttggttttcacatttgtcagatggaaatgattgtgatttctcagggctgttgagcaggtaaatgaaaatgttttatgaaagaaagcaccaagtttcattttggtcttagcccttgctatgtccctagcaagaagtagatattcatagggatattttgtttgatgtgaggagttcttacagcaagagcttgtagaaggccaaaagcttctggattctawtcccaaaagcaggagatgacagtgacagggtggttttggtgaggagagatgaggtagaaaatgagtgcaarcccgctggcyactgaccccatggctcgcccacagatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaaggtgtgtaccagttgttttcccttccccttttcctcstccccgatactctggtctcacccaggatcttcctcctcctacagggacctctcccaatgttgctcgtaatgccattgtcaactgtgctgagctrgtgacctatgacctcatcaaggatgccctcctgaaagccaacctcatgacaggtgagtcatgaggtagayggtgctgggtctcacccttcccccatgccaggagcaggtgcgggggtctrgctgacaccagaagaccacatcttttcatcctatttgccctttgcagggagagtaagatatctcttacttgccatattgaagccaattgggatgaagctcccactttgcacattgaggaactgaggctagattggcaaaatgactctttcaggtcctcagaagatgtctcagctggagtccctgtctgtttttgtttttttgtttgtttgttttttgttttttttgagatagagtctcactctgttacccgtgtaatctcagctcactgcaaccttctcctcctgggttcaagcgattcttgtgcctcagcctcccgagtagctgggatgacaggtgtgcaccagcacactggctaatttttgtatttttagtagagatggagtttcaccatgttagccaggctggtctcgaactcctggcctcaagtgatctgcccaccttggcctcccaatgtgctgggattacaggtgtgagcctctgcgccccatcctcttgtttgttttttgagacagggtcttgctcggttgcccaggctggagtgcagtggggtgattaatggctcattgcagcctcgacctccctgactcaagcaatcctcccacctcagcctcctgagtagctggggctgactacaggcatgcacactgtgcctggctaatttttgtattttgtagagacagggtttttgccatgttacccagtctggtcttgaactcctgggctcaagtgatccacccacctcggcctccaaaagtcctggattacaggcatgagacattgtgcccagcctctctgtctctttaaaatcatgaaaactcgtagctacttaagtaattctcctgccttctggaatgatgggtgaagatcttgactgccttgcctgctcctccttggcagatgacctcccttgccacttcatttctgcctttggggcaggcttctgcaccactgtcatcgcctcccctgtagacgtggtcaagacgagatacatgaactctgccctgggccagtayagtagcgctggccactgtgcccttaccatgctccagaaggaggggccccgagccttctacaaagggtgagcctctggtcctccccacccagttcaggcctcttggctatgcatgtctattrtgggtgggagagaaccacctggaagtgagtagcagccaagtgtgactatttctgatcctggtcctggcatttcaccagcattcacctatccccttaattccttcctcccagaattgctaccatcactgtttattaggtgttaaatggagactcaaagggaattcatgcttatagccaagcagctgtgagctcagttcattgagtcctcccagcctcctttgggacagagcaactgggttggattgaataccaggcccagtgagggaagtgggaggtggaggtgcccccatgacctgtgatttttctcctctaggttcatgccctcctttctccgcttgggttcctggaacgtggtgatgttcgtcacctatgagcagctgaaacgagccctcatggctgcctgcacttcccgagaggctcccttctgagcctctcctgctgctgacctgatcaccwctggctttgtctctagccgggccatgctttccttttcttccttctttctcttccctccttcccttctctccttccctctttccccacctcttccttccgctcctttacctaccaccttccctctttctacattctcatctactcattgtctcagtgctggtggagttgacatttgacagtgtgggaggcctcgtaccagccaggatcccaagcgtcccgtcccttggaaagttcagccagaatcttcgtcctgcccccgacagcccagcctagcccacttgtcatccataaagcaagctcaaccttggcgtctcctccctctcttgtagctcttaccagaggtcttggtccaatggcctttttggtacctggtgggcaggggaggaaccacctgactttgaaaatgggtgtgatccaccttccacctccagcatccaatctgaagcccgtgtaggtcatctggtccatttctctctagacccaggccctgtactaacatggggagtgcaggagccacctgagagacagcagtgcctccccttcctttgccgggccacttgagctcttactcagaatctggtactctagtgcctgccatcccaaccccccacccacaccgcaggcctgtttatctgca

SEQ ID NO: 2 - DNA, 930 bp, Homo sapiens
atggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcatttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctga

SEQ ID NO: 3 - protein, 309 aa, Homo sapiens [sequence omitted]

SEQ ID NO: 4 - DNA, 15 bp, Homo sapiens
aactgctsagctcaa

SEQ ID NO: 5 - DNA, 15 bp, Homo sapiens
ctctgtcycatcgtg

SEQ ID NO: 6 - DNA, 15 bp, Homo sapiens
ttaggtgyttcgtct

SEQ ID NO: 7 - DNA, 15 bp, Homo sapiens
tgaagaaygggacac

SEQ ID NO: 8 - DNA, 15 bp, Homo sapiens
ccccaccsccgacag

SEQ ID NO: 9 - DNA, 15 bp, Homo sapiens
aggagaaractgagg

SEQ ID NO: 10 - DNA, 15 bp, Homo sapiens
taagcctrtgggtct

SEQ ID NO: 11 - DNA, 15 bp, Homo sapiens
gcctgttrggtctta

SEQ ID NO: 12 - DNA, 15 bp, Homo sapiens
ttttctcyacttctg

SEQ ID NO: 13 - DNA, 15 bp, Homo sapiens
tctgcatmcagcaga

SEQ ID NO: 14 - DNA, 15 bp, Homo sapiens
ggagcccwtcatgaa

SEQ ID NO: 15 - DNA, 15 bp, Homo sapiens
ttctcagkgatgatt

SEQ ID NO: 16 - DNA, 15 bp, Homo sapiens
gattctawtcccaaa

SEQ ID NO: 17 - DNA, 15 bp, Homo sapiens
agtgcaarcccgctg

SEQ ID NO: 18 - DNA, 15 bp, Homo sapiens
cgctggcyactgacc

SEQ ID NO: 19 - DNA, 15 bp, Homo sapiens
tttcctcstccccga

SEQ ID NO: 20 - DNA, 15 bp, Homo sapiens
ctgagctrgtgacct

SEQ ID NO: 21 - DNA, 15 bp, Homo sapiens
aggtagayggtgctg

SEQ ID NO: 22 - DNA, 15 bp, Homo sapiens
ggggtctrgctgaca

SEQ ID NO: 23 - DNA, 15 bp, Homo sapiens
gccagtayagtagcg

SEQ ID NO: 24 - DNA, 15 bp, Homo sapiens
gtctattrtgggtgg

SEQ ID NO: 25 - DNA, 15 bp, Homo sapiens
gatcaccwctggctt

SEQ ID NO: 26 - DNA, 15 bp, Homo sapiens
gcctcgaactgctsa

SEQ ID NO: 27 - DNA, 15 bp, Homo sapiens
gattgcttgagctsa

SEQ ID NO: 28 - DNA, 15 bp, Homo sapiens
gagagtctctgtcyc

SEQ ID NO: 29 - DNA, 15 bp, Homo sapiens
gggggtcacgatgrg

SEQ ID NO: 30 - DNA, 15 bp, Homo sapiens
tgaccattaggtgyt

SEQ ID NO: 31 - DNA, 15 bp, Homo sapiens
ggtgggagacgaarc

SEQ ID NO: 32 - DNA, 15 bp, Homo sapiens
cagctttgaagaayg

SEQ ID NO: 33 - DNA, 15 bp, Homo sapiens
ctaaaggtgtcccrt

SEQ ID NO: 34 - DNA, 15 bp, Homo sapiens
taccatccccaccsc

SEQ ID NO: 35 - DNA, 15 bp, Homo sapiens
acttcactgtcggsg

SEQ ID NO: 36 - DNA, 15 bp, Homo sapiens
ttataaaggagaara

SEQ ID NO: 37 - DNA, 15 bp, Homo sapiens
cgtgggcctcagtyt

SEQ ID NO: 38 - DNA, 15 bp, Homo sapiens
agaacataagcctrt

SEQ ID NO: 39 - DNA, 15 bp, Homo sapiens
aggcacagacccaya

SEQ ID NO: 40 - DNA, 15 bp, Homo sapiens
gtctgtgcctgttrg

SEQ ID NO: 41 - DNA, 15 bp, Homo sapiens
ccagactaagaccya

SEQ ID NO: 42 - DNA, 15 bp, Homo sapiens
tgaaacttttctcya

SEQ ID NO: 43 - DNA, 15 bp, Homo sapiens
agctgacagaagtrg

SEQ ID NO: 44 - DNA, 15 bp, Homo sapiens
gaaatctctgcatmc

SEQ ID NO: 45 - DNA, 15 bp, Homo sapiens
cctttgtctgctgka

SEQ ID NO: 46 - DNA, 15 bp, Homo sapiens
agggagggagcccwt

SEQ ID NO: 47 - DNA, 15 bp, Homo sapiens
caatacttcatgawg

SEQ ID NO: 48 - DNA, 15 bp, Homo sapiens
ccttttttctcagkg

SEQ ID NO: 49 - DNA, 15 bp, Homo sapiens
aagatcaatcatcmc

SEQ ID NO: 50 - DNA, 15 bp, Homo sapiens
cttctggattctawt

SEQ ID NO: 51 - DNA, 15 bp, Homo sapiens
cctgcttttgggawt

SEQ ID NO: 52 - DNA, 15 bp, Homo sapiens
aaaatgagtgcaarc

SEQ ID NO: 53 - DNA, 15 bp, Homo sapiens
agtggccagcgggyt

SEQ ID NO: 54 - DNA, 15 bp, Homo sapiens
caagcccgctggcya

SEQ ID NO: 55 - DNA, 15 bp, Homo sapiens
ccatggggtcagtrg

SEQ ID NO: 56 - DNA, 15 bp, Homo sapiens
tccccttttcctcst

SEQ ID NO: 57 - DNA, 15 bp, Homo sapiens
agagtatcggggasg

SEQ ID NO: 58 - DNA, 15 bp, Homo sapiens
actgtgctgagctrg

SEQ ID NO: 59 - DNA, 15 bp, Homo sapiens
ggtcataggtcacya

SEQ ID NO: 60 - DNA, 15 bp, Homo sapiens
gtcatgaggtagayg

SEQ ID NO: 61 - DNA, 15 bp, Homo sapiens
gagacccagcaccrt

SEQ ID NO: 62 - DNA, 15 bp, Homo sapiens
ggtgcgggggtctrg

SEQ ID NO: 63 - DNA, 15 bp, Homo sapiens
ttctggtgtcagcya

SEQ ID NO: 64 - DNA, 15 bp, Homo sapiens
ccctgggccagtaya

SEQ ID NO: 65 - DNA, 15 bp, Homo sapiens
ggccagcgctactrt

SEQ ID NO: 66 - DNA, 15 bp, Homo sapiens
atgcatgtctattrt

SEQ ID NO: 67 - DNA, 15 bp, Homo sapiens
tctctcccacccaya

SEQ ID NO: 68 - DNA, 15 bp, Homo sapiens
tgacctgatcaccwc

SEQ ID NO: 69 - DNA, 15 bp, Homo sapiens
gagacaaagccagwg

SEQ ID NO: 70 - DNA, 10 bp, Homo sapiens
tcgaactgct

SEQ ID NO: 71 - DNA, 10 bp, Homo sapiens
tgcttgagct

SEQ ID NO: 72 - DNA, 10 bp, Homo sapiens
agtctctgtc

SEQ ID NO: 73 - DNA, 10 bp, Homo sapiens
ggtcacgatg

SEQ ID NO: 74 - DNA, 10 bp, Homo sapiens
ccattaggtg

SEQ ID NO: 75 - DNA, 10 bp, Homo sapiens
gggagacgaa

SEQ ID NO: 76 - DNA, 10 bp, Homo sapiens
ctttgaagaa

SEQ ID NO: 77 - DNA, 10 bp, Homo sapiens
aaggtgtccc

SEQ ID NO: 78 - DNA, 10 bp, Homo sapiens
catccccacc

SEQ ID NO: 79 - DNA, 10 bp, Homo sapiens
tcactgtcgg

SEQ ID NO: 80 - DNA, 10 bp, Homo sapiens
taaaggagaa

SEQ ID NO: 81 - DNA, 10 bp, Homo sapiens
gggcctcagt

SEQ ID NO: 82 - DNA, 10 bp, Homo sapiens
acataagcct

SEQ ID NO: 83 - DNA, 10 bp, Homo sapiens
cacagaccca

SEQ ID NO: 84 - DNA, 10 bp, Homo sapiens
tgtgcctgtt

SEQ ID NO: 85 - DNA, 10 bp, Homo sapiens
gactaagacc

SEQ ID NO: 86 - DNA, 10 bp, Homo sapiens
aacttttctc

SEQ ID NO: 87 - DNA, 10 bp, Homo sapiens
tgacagaagt

SEQ ID NO: 88 - DNA, 10 bp, Homo sapiens
atctctgcat

SEQ ID NO: 89 - DNA, 10 bp, Homo sapiens
ttgtctgctg

SEQ ID NO: 90 - DNA, 10 bp, Homo sapiens
gagggagccc

SEQ ID NO: 91 - DNA, 10 bp, Homo sapiens
tacttcatga

SEQ ID NO: 92 - DNA, 10 bp, Homo sapiens
tttttctcag

SEQ ID NO: 93 - DNA, 10 bp, Homo sapiens
atcaatcatc

SEQ ID NO: 94 - DNA, 10 bp, Homo sapiens
ctggattcta

SEQ ID NO: 95 - DNA, 10 bp, Homo sapiens
gcttttggga

SEQ ID NO: 96 - DNA, 10 bp, Homo sapiens
atgagtgcaa

SEQ ID NO: 97 - DNA, 10 bp, Homo sapiens
ggccagcggg

SEQ ID NO: 98 - DNA, 10 bp, Homo sapiens
gcccgctggc

SEQ ID NO: 99 - DNA, 10 bp, Homo sapiens
tggggtcagt

SEQ ID NO: 100 - DNA, 10 bp, Homo sapiens
ccttttcctc

SEQ ID NO: 101 - DNA, 10 bp, Homo sapiens
gtatcgggga

SEQ ID NO: 102 - DNA, 10 bp, Homo sapiens
gtgctgagct

SEQ ID NO: 103 - DNA, 10 bp, Homo sapiens
cataggtcac

SEQ ID NO: 104 - DNA, 10 bp, Homo sapiens
atgaggtaga

SEQ ID NO: 105 - DNA, 10 bp, Homo sapiens
acccagcacc

SEQ ID NO: 106 - DNA, 10 bp, Homo sapiens
gcgggggtct

SEQ ID NO: 107 - DNA, 10 bp, Homo sapiens
tggtgtcagc

SEQ ID NO: 108 - DNA, 10 bp, Homo sapiens
tgggccagta

SEQ ID NO: 109 - DNA, 10 bp, Homo sapiens
cagcgctact

SEQ ID NO: 110 - DNA, 10 bp, Homo sapiens
catgtctatt

SEQ ID NO: 111 - DNA, 10 bp, Homo sapiens
ctcccaccca

SEQ ID NO: 112 - DNA, 10 bp, Homo sapiens
cctgatcacc

SEQ ID NO: 113 - DNA, 10 bp, Homo sapiens
acaaagccag

SEQ ID NO: 114 - DNA, 18 bp, Homo sapiens
tgtaaaacgacggccagt

SEQ ID NO: 115 - DNA, 19 bp, Homo sapiens
aggaaacagctatgaccat

SEQ ID NO: 116 - DNA, 2760 bp, Homo sapiens
  allele (30)..(30): PS1 polymorphic base cytosine or guanine
catgttggccaggctggcctcgaactgctsagctcaagcaatccgcccgcctcggcctcannnnnnnnnnnnnnnnnnnnnnnnnnnnnnnnnnnnnnnnnnnnnnnnnnnnnnnnnnnnacctagccaattcccggagagtctctgtcycatcgtgaccccctcacaactctcccactcnnnnnnnnnnnnnnnnnnnnnnnnnnnnnnnnnnnnnnnnnnnnnnnnnnnnnnnnnnnncacaggacacatagtatgaccattaggtgyttcgtctcccacccattttctatggaaaacnnnnnnnnnnnnnnnnnnnnnnnnnnnnnnnnnnnnnnnnnnnnnnnnnnnnnnnnnnnnccatgatagccactggcagctttgaagaaygggacacctttagagaagcttgatcttggannnnnnnnnnnnnnnnnnnnnnnnnnnnnnnnnnnnnnnnnnnnnnnnnnnnnnnnnnnncattaacaaaaataattaccatccccaccsccgacagtgaagtggctctttccagttcacnnnnnnnnnnnnnnnnnnnnnnnnnnnnnnnnnnnnnnnnnnnnnnnnnnnnnnnnnnnngatcttggtccccattttataaaggagaaractgaggcccacgtgtaacagtgattggccnnnnnnnnnnnnnnnnnnnnnnnnnnnnnnnnnnnnnnnnnnnnnnnnnnnnnnnnnnnntcctgcttagacatccagaacataagcctrtgggtctgtgcctgttgggtcttagtctggnnnnnnnnnnnnnnnnnnnnnnnnnnnnnnnnnnnnnnnnnnnnnnnnnnnnnnnnnnnngaacataagcctatgggtctgtgcctgttrggtcttagtctgggtgaaacttttctctacnnnnnnnnnnnnnnnnnnnnnnnnnnnnnnnnnnnnnnnnnnnnnnnnnnnnnnnnnnnntgggtcttagtctgggtgaaacttttctcyacttctgtcagctctccagatgaaccacagnnnnnnnnnnnnnnnnnnnnnnnnnnnnnnnnnnnnnnnnnnnnnnnnnnnnnnnnnnnntgtgggcatcatcagtgaaatctctgcatmcagcagacaaagggctggtccagtggctgtnnnnnnnnnnnnnnnnnnnnnnnnnnnnnnnnnnnnnnnnnnnnnnnnnnnnnnnnnnnnctctccctgcagtgaaagggagggagcccwtcatgaagtattgactgcttgagcaggaatnnnnnnnnnnnnnnnnnnnnnnnnnnnnnnnnnnnnnnnnnnnnnnnnnnnnnnnnnnnnagaaagtcaggggccagtgcgcgctacagycagcgcccagtaccgcggtgtgatgggcacnnnnnnnnnnnnnnnnnnnnnnnnnnnnnnnnnnnnnnnnnnnnnnnnnnnnnnnnnnnngtgtaggccccttggcccttttttctcagkgatgattgatcttagttcattcagccatatnnnnnnnnnnnnnnnnnnnnnnnnnnnnnnnnnnnnnnnnnnnnnnnnnnnnnnnnnnnntgtagaaggccaaaagcttctggattctawtcccaaaagcaggagatgacagtgacagggnnnnnnnnnnnnnnnnnnnnnnnnnnnnnnnnnnnnnnnnnnnnnnnnnnnnnnnnnnnnaggagagatgaggtagaaaatgagtgcaarcccgctggccactgaccccatggctcgcccnnnnnnnnnnnnnnnnnnnnnnnnnnnnnnnnnnnnnnnnnnnnnnnnnnnnnnnnnnnnaggtagaaaatgagtgcaagcccgctggcyactgaccccatggctcgcccacagatgccannnnnnnnnnnnnnnnnnnnnnnnnnnnnnnnnnnnnnnnnnnnnnnnnnnnnnnnnnnnaccagttgttttcccttccccttttcctcstccccgatactctggtctcacccaggatctnnnnnnnnnnnnnnnnnnnnnnnnnnnnnnnnnnnnnnnnnnnnnnnnnnnnnnnnnnnncgtaatgccattgtcaactgtgctgagctrgtgacctatgacctcatcaaggatgccctcnnnnnnnnnnnnnnnnnnnnnnnnnnnnnnnnnnnnnnnnnnnnnnnnnnnnnnnnnnnnacctcatgacaggtgagtcatgaggtagayggtgctgggtctcacccttcccccatgccannnnnnnnnnnnnnnnnnnnnnnnnnnnnnnnnnnnnnnnnnnnnnnnnnnnnnnnnnnnccccatgccaggagcaggtgcgggggtctrgctgacaccagaagaccacatcttttcatcnnnnnnnnnnnnnnnnnnnnnnnnnnnnnnnnnnnnnnnnnnnnnnnnnnnnnnnnnnnnagatacatgaactctgccctgggccagtayagtagcgctggccactgtgcccttaccatgnnnnnnnnnnnnnnnnnnnnnnnnnnnnnnnnnnnnnnnnnnnnnnnnnnnnnnnnnnnnttcaggcctcttggctatgcatgtctattrtgggtgggagagaaccacctggaagtgagtnnnnnnnnnnnnnnnnnnnnnnnnnnnnnnnnnnnnnnnnnnnnnnnnnnnnnnnnnnnngagcctctcctgctgctgacctgatcaccwctggctttgtctctagccgggccatgctttnnnnnnnnnnnnnnnnnnnnnnnnnnnnnnnnnnnnnnnnnnnnnnnnnnnnnnnnnnnn